SEQUENCE LISTING (39 sequences)

SEQ ID NO: 1 - DNA, 2878 bp, human
ccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggccgcgggacggataacaatttcacacaggaaacagctatgaccattaggcctatttaggtgacactatagaacaagtttgtacaaaaaagcaggctggtaccggtccggaattcccgggaggaggaggaggtggcggcgagaagatggcgacttcgaacaatccgcggaaattcagcgagaagatcgcgctgcacaatcagaagcaggcggaggagacggcggccttcgaggaggtcatgaaggacctgagcctgacgcgggccgcgcggctccagctccagaaatcccagtacctgcaactgggccccagccgaggccagtactatggcgggtccctgcccaacgtgaaccagatcgggagtggcaccatggacctgcccttccagcccagcggatttctgggggaggccctggcagcggctcctgtctctctgacccccttccaatcctcgggcctggacaccagccggaccacccggcaccatgggctggtggacagggtgtaccgggagcgtggccggctcggctccccacaccgccggcccctgtcagtggacaaacacggacggcaggccgacagctgcccctatggcaccatgtacctctcaccacccgcggacaccagctggagaaggaccaattctgactccgccctgcaccagagcacaatgacgcccacgcagccagaatcctttagcagtgggtcccaggacgtgcaccagaaaagagtcttactgttaacagtcccaggaatggaagagaccacatcagaggcagacaaaaacctttccaagcaagcatgggacaccaagaagacggggtccaggcccaagtcctgtgaggtccccggaatcaacatcttcccgtctgccgaccaggaaaacactacagccctgatccccgccacccacaacacaggggggtccctgcccgacctgaccaacatccacttcccctccccgctcccgaccccgctggaccccgaggagcccaccttccctgcactgagcagctccagcagcaccggcaacctcgcggccaacctgacgcacctgggcatcggtggcgccggccagggaatgagcacacctggctcctctccacagcaccgcccagctggcgtcagccccctgtccctgagcacagaggcaaggcgtcagcaggcatcgcccaccctgtccccgctgtcacccatcactcaggctgtagccatggacgccctgtctctggagcagcagctgccctacgccttcttcacccaggcgggctcccagcagccaccgccgcagccccagcccccgccgcctcctccacccgcgtcccagcagccaccacccccgccacccccacaggcgcccgtccgcctgccccctggtggccccctgttgcccagcgccagcctgactcgtgggccacagccgcccccgcttgcagtcacggtaccgtcctctctcccccagtcccccccagagaaccctggccagccatcgatggggatcgacatcgcctcggcgccggctctgcagcagtaccgcactagcgccggctccccggccaaccagtctcccacctcgccagtctccaatcaaggcttctccccagggagctccccgcaacacacttccaccctgggcagcgtgtttggggacgcgtactatgagcagcagatggcggccaggcaggccaatgctctgtcccaccagctggagcagttcaacatgatggagaacgccatcagctccagcagcctgtacagcccgggctccacactcaactactcgcaggcggccatgatgggcctcacgggcagccacgggagcctgccggactcgcagcaactgggatacgccagccacagtggcatccccaacatcatcctcacagtgacaggagagtccccccccagcctctctaaagaactgaccagctctctggccggggtcggcgacgtcagcttcgactccgacagccagtttcccctggacgaactcaagatcgaccccctgaccctcgacggactgcacatgctcaacgaccccgacatggttctggccgacccagccaccgaggacaccttccggatggaccgcctgtgagcgggcacgccggcaccctgccgctcagccgtcccgacggcgcctccccagcccggggacggccgtgctccgtccctcgccaacggccgagcttgtgattctgagcttgcaatgccgccaagcgccccccgccagcccgcccccggttgtccacctcccgcgaagcccaatcgcgaggccgcgagccgggccgtccacccacccgcccgcccagggctgggctgggatcggaggccgtgagcctcccgcccctgcagaccctccctgcactggctccctcgcccccagccccggggcctgagccgtcccctgtaagatgcgggaagtgtcagctcccggcgtggcgggcaggctcaggggaggggcgcgcatggtccgccagggctgtgggccgtggcgcattttccgactgtttgtccagctctcactgccttccttggttcccggtcccccagcccatccgccatccccagcccgtggtcaggtagagagtgagccccacgccgccccagggaggaggcgccagagcgcggggcagacgcaaagtgaaataaacactattttgacggcaaaaaaaaaaaaaaagggcggccgctctagagtatccctcgaggggcccaag

SEQ ID NO: 2 - protein, 650 aa, human
MATSNNPRKFSEKIALHNQKQAEETAAFEEVMKDLSLTRAARLQLQKSQYLQLGPSRGQYYGGSLPNVNQIGSGTMDLPFQPSGFLGEALAAAPVSLTPFQSSGLDTSRTTRHHGLVDRVYRERGRLGSPHRRPLSVDKHGRQADSCPYGTMYLSPPADTSWRRTNSDSALHQSTMTPTQPESFSSGSQDVHQKRVLLLTVPGMEETTSEADKNLSKQAWDTKKTGSRPKSCEVPGINIFPSADQENTTALIPATHNTGGSLPDLTNIHFPSPLPTPLDPEEPTFPALSSSSSTGNLAANLTHLGIGGAGQGMSTPGSSPQHRPAGVSPLSLSTEARRQQASPTLSPLSPITQAVAMDALSLEQQLPYAFFTQAGSQQPPPQPQPPPPPPPASQQPPPPPPPQAPVRLPPGGPLLPSASLTRGPQPPPLAVTVPSSLPQSPPENPGQPSMGIDIASAPALQQYRTSAGSPANQSPTSPVSNQGFSPGSSPQHTSTLGSVFGDAYYEQQMAARQANALSHQLEQFNMMENAISSSSLYSPGSTLNYSQAAMMGLTGSHGSLPDSQQLGYASHSGIPNIILTVTGESPPSLSKELTSSLAGVGDVSFDSDSQFPLDELKIDPLTLDGLHMLNDPDMVLADPATEDTFRMDRL

SEQ ID NO: 3 - DNA, 32 bp, Artificial Sequence
  primer
gcccaagctttgtgctctgctgtctctgaaag

SEQ ID NO: 4 - DNA, 23 bp, Artificial Sequence
  primer
gccctgaggggatgggccatcag

SEQ ID NO: 5 - DNA, 36 bp, Artificial Sequence
  primer
cgcggatccgaagtgtgatgactcaggtttgccctg

SEQ ID NO: 6 - DNA, 36 bp, Artificial Sequence
  primer
cgcggatccgaagtgtgatatctcaggtttgccctg

SEQ ID NO: 7 - DNA, 54 bp, Artificial Sequence
  primer
gccctgaggggatgggccatcagttgcaaatcgttaactttcctctgacataat

SEQ ID NO: 8 - DNA, 39 bp, Artificial Sequence
  primer
gccctgaggggatgggccatcagctacgagtcgtggaat

SEQ ID NO: 9 - DNA, 47 bp, Artificial Sequence
  primer
cgcggatccgaagtgtgatgactcaggtttgccctgaggggatgggc

SEQ ID NO: 10 - DNA, 43 bp, Artificial Sequence
  primer
cagttgcaaatcgtggaatttcctctcgatcaatgaaaagatg

SEQ ID NO: 11 - DNA, 39 bp, Artificial Sequence
  primer
gccctgaggggatgggccatcagttgcaaatcgtggaat

SEQ ID NO: 12 - DNA, 19 bp, Artificial Sequence
  primer
cgcctggtaccgagctctg

SEQ ID NO: 13 - DNA, 19 bp, Artificial Sequence
  primer
acccaagatctcgagcccg

SEQ ID NO: 14 - DNA, 99 bp, Artificial Sequence
  primer
cgcctggtaccgagctctgacataatgacataatgacataatgacataatgacataatgacataattacgcgtgctagcccgggctcgagatcttgggt

SEQ ID NO: 15 - DNA, 2520 bp, human
  modified_base (0)...(0)
  misc_feature 2, 13, 2431, 2453, 2465, 2468, 2469, 2479, 2488, 2489,2492, 2505, 2512, 2514, 2519, 2520: n = A,T,C or G
antttttgtacanaaaagcaggctgttaccggtccggattcccgggatctaggctggggccgggttcgcggtgctcgctgaggcggcggtggctacggctggaggagccgggccgaggccgcggcggaggccgcggctggtactgggagggtggcagggagggacggggaaggaagatggcgacgtcgggggcgaacgggcctggttcggccacggcctcggcttccaatccgcgcaaatttagtgagaagattgcgctgcagaagcagcgtcaggccgaggagacggcggccttcgaggaggtgatgatggacatcggctccacccggttacaggcccaaaaactgcgactggcatacacaaggagctctcattatggtgggtctctgcccaatgttaaccagattggctctggcctggccgagttccagagccccctccactcacctttggattcatctcggagcactcggcaccatgggctggtggaacgggtgcagcgagatcctcgaagaatggtgtccccacttcgccgatacacccgccacattgacagctctccctatagtcctgcctacttatctcctcccccagagtctagctggcgaaggacgatggcctggggcaatttccctgcagagaaggggcagttgtttcgactaccatctgcacttaacaggacaagctctgactctgcccttcatacaagtgtgatgaaccccagtccccaggatacctacccaggccccacacctcccagcatcctgcccagccgacgtgggggtattctggatggtgaaatggaccccaaagtacctgctattgaggagaacttgctagatgacaagcatttgctgaagccatgggatgctaagaagctatcctcatcctcttcccgacctcggtcctgtgaagtccctggaattaacatctttccatctcctgaccagcctgccaatgtgcctgtcctcccacctgccatgaacacggggggctccctacctgacctcaccaacctgcactttcccccaccactgcccacccccctggaccctgaagagacagcctaccctagcctgagtgggggcaacagtacctccaatttgacccacaccatgactcacctgggcatcagcagggggcatgggcctgggcccggctatgatgcaccaggacttcattcacctctcagccacccatccctgcagtcctccctaagcaatcccaacctccaggcttccctgagcagtcctcagccccagcttcagggctcccacagccacccctctctgcctgcctcctccttggcctgccatgtactgcccaccacctccctgggccacccctcactcagtgctccggctctctcctcctcctcttcctcctcctccacttcatctcctgttttgggcgccccctcttaccctgcttctacccctggggcctccccccaccaccgccgtgtgcccctcagccccctgagtttgctcgcgggcccagccgacgccagaaggtcccaacagcagctgcccaaacagttttcgccaacaatgtcacccaccttgtcttccatcactcagggcgtccccctggataccagtaaactgtccactgaccagcggttacccccctacccatacagctccccaagtctggttctgcctacccagccccacaccccaaagtctctacagcagccagggctgccctctcagtcttgttcagtgcagtcctcaggtgggcagcccccaggcaggcagtctcattatgggacaccgtacccacctgggcccagtgggcatgggcaacagtcttaccaccggccaatgagtgacttcaacctggggaatctggagcagttcagcatggagagcccatcagccagcctggtgctggatccccctggcttttctgaagggcctggatttttagggggtgaggggccaatgggtggcccccaggatccccacaccttcaaccaccagaacttgacccactgttcccgccatggctcagggcctaacatcatcctcacaggggactcctctccaggtttctctaaggagattgcagcagccctggccggagtgcctggctttgaggtgtcagcagctggattggagctagggcttgggctagaagatgagctgcgcatggagccactgggcctggaagggctaaacatgctgagtgacccctgtgccctgctgcctgatcctgctgtggaggagtcattccgcagtgaccggctccaatgagggcacctcatcaccatccctcttcttggccccatcccccaccaccattcctttcctcccttccccctggcaggtagagactctactctctgtccccagatcctctttctagcatgaatgaaggatgccaagaatgagaaaaagcaaggggtttgtccaggtggcccctgaantctgcgcaagggatgggcctgnggggaacctcanggnnagggcccaanggccacttnnaanctttgaaccgtcngtctggnanggtcnn

SEQ ID NO: 16 - protein, 693 aa, human [sequence omitted]

SEQ ID NO: 17 - DNA, 17 bp, human
ccgtcatttcaccaagc

SEQ ID NO: 18 - protein, 7 aa, human
EETRAFE

SEQ ID NO: 19 - protein, 7 aa, unknown
  predicted protein
EETAAFE

SEQ ID NO: 20 - DNA, 34 bp, Artificial Sequence
  primer
ccggaattcgccatggccgcctcgccgggctcgg

SEQ ID NO: 21 - DNA, 44 bp, Artificial Sequence
  primer
ccgcgacagggtgaggtcggtcatgagctgctcgaaggcccgcg

SEQ ID NO: 22 - DNA, 44 bp, Artificial Sequence
  primer
gaagcttctgaaattgaacccgcgacagggtgaggtcggtcatg

SEQ ID NO: 23 - DNA, 63 bp, Artificial Sequence
  primer
tggtaaggatcctccatggtactgtgtaaggcgcagttgctgaagcttctgaaattgaacccg

SEQ ID NO: 24 - DNA, 2259 bp, human
  misc_feature 1, 13: n = A,T,C or G
nttttttgtacanaaaagcaggctgttaccggtccggaattcgccatggccgcctcgccgggctcgggcagcgccaacccgcggaagttcagtgagaagatcgcgctgcacacgcagagacaggccgaggagacgcgggccttcgagcagctcatgaccgacctcaccctgtcgcgggttcaatttcagaagcttcagcaactgcgccttacacagtaccatggaggatccttaccaaatgtgagccagctgcggagcaatgcgtcagagtttcagccgtcatttcaccaagctgataatgttcggggaacccgccatcacgggctggtggagaggccatccaggaaccgcttccaccccctccaccgaaggtctggggacaagccagggcgacaatttgatggtagtgcttttggagccaattattcctcacagcctctggatgagagttggccaaggcagcagcctccttggaaagacgaaaagcatcctgggttcaggctgacatctgcacttaacaggaccaattctgattctgctcttcacacgagtgctctgagtaccaagccccaggacccctatggaggagggggccagtcggcctggcctgccccatacatggggttttgtgatggtgagaataatggacatggggaagtagcatctttccctggcccattgaaagaagagaatctgttaaatgttcctaagccactgccaaaacaactgtgggagaccaaggagattcagtccctgtcaggacgccctcgatcctgtgatgttggaggtggcaatgcttttccacataatggtcaaaacctaggcctctcacccttcttggggactttgaacactggagggtcattgccagatctaaccaacctccactactcgacacccctgccagcctccctggacaccaccgaccaccactttggcagtatgagtgtggggaatagtgtgaacaacatcccagctgctatgacccacctgggtataagaagctcctctggtctccagagttctcggagtaacccctccatccaagccacgctcaataagactgtgctttcctcttccttaaataaccacccacagacatctgttcccaacgcatctgctcttcacccttcgctccgtctgttttcccttagcaacccatctctttccaccacaaacctgagcggcccgtctcgccgtcggcagcctcccgtcagccctctcacgctttctcctggccctgaagcacatcaaggtttcagcagacagctgtcttcaaccagcccactggccccatatcctacctcccagatggtgtcctcagaccgaagccaactttcctttctgcccacagaagctcaagcccaggtgtcgccgccacccccttaccctgcaccccaggagctcacccagcccctcctgcagcagccccgcgcccctgaggcccctgcccagcagccccaggcagcctcctcactgccacagtcagactttcagcttctcccggcccagggctcatctttgaccaacttcttcccagatgtgggttttgaccagcagtccatgaggccaggccctgcctttcctcaacaggtgcctctggtgcaacaaggttcccgagaactgcaggactcttttcatttgagaccaagcccgtattccaactgcgggagtctcccgaacaccatcctgccagaagactccagcaccagcctgttcaaagacctcaacagtgcgctggcaggcctgcctgaggtcagcctgaacgtggacactccatttccactggaagaggagctgcagattgaacccctgagcctggatggactcaacatgttaagtgactccagcatgggcctgctggacccctctgttgaagagacgtttcgagctgacagactgtgaacagaaggcagtggaacagaagaatgtttttctgcaacagccaaaatagaatggaatagaatgaagccagctgataccacgggctttcgttatcttgacatagaaggaagcagtgccacggctccagggtttcagatgagatcccatctcagacactgtggcttcctccagatcacacagctttgtactgcctctcccgcctgtggccaaagtcgtgttgcagcaggcaggctgcttggagcttcccatgaactggaaagctcacctccactgcatctttttactggccatccagtcagccgatgtgtaagagtaggaaatactgtgtcactggaggccctccgtagcattggg

SEQ ID NO: 25 - protein, 619 aa, human
MAASPGSGSANPRKFSEKIALHTQRQAEETRAFEQLMTDLTLSRVQFQKLQQLRLTQYHGGSLPNVSQLRSNASEFQPSFHQADNVRGTRHHGLVERPSRNRFHPLHRRSGDKPGRQFDGSAFGANYSSQPLDESWPRQQPPWKDEKHPGFRLTSALNRTNSDSALHTSALSTKPQDPYGGGGQSAWPAPYMGFCDGENNGHGEVASFPGPLKEENLLNVPKPLPKQLWETKEIQSLSGRPRSCDVGGGNAFPHNGQNLGLSPFLGTLNTGGSLPDLTNLHYSTPLPASLDTTDHHFGSMSVGNSVNNIPAAMTHLGIRSSSGLQSSRSNPSIQATLNKTVLSSSLNNHPQTSVPNASALHPSLRLFSLSNPSLSTTNLSGPSRRRQPPVSPLTLSPGPEAHQGFSRQLSSTSPLAPYPTSQMVSSDRSQLSFLPTEAQAQVSPPPPYPAPQELTQPLLQQPRAPEAPAQQPQAASSLPQSDFQLLPAQGSSLTNFFPDVGFDQQSMRPGPAFPQQVPLVQQGSRELQDSFHLRPSPYSNCGSLPNTILPEDSSTSLFKDLNSALAGLPEVSLNVDTPFPLEEELQIEPLSLDGLNMLSDSSMGLLDPSVEETFRADRL

SEQ ID NO: 26 - DNA, 2992 bp, drosophila melanogaster
atggccaatccgcgcaagttcagcgagaagatcgctctgcagaagcagaagcaggcggagggcacagcggaattcgagcggatcatgaaggaggtgtatgccacgaagagggatgagccgcctgcgaatcagaagatcctagacggccttgtcggcggtcaggaggtaagccaatcctcgccaggcgcaggcaatgggacgggcggaggtggcagtggttccggcagtggagccagcggcggaggagcctcaccagatggcctgggaggcggcggtggttctccgacggcttatcgagaatcccgagggcgcagcgtaggtgtgggtcccatgcgaagaccgtcggagcgcaagcaggatcgttcgccctacggcagcagcagtacgcaacaaaccttagacaacggccagctaaatccgcatcttcttggtccacctacggcggagagtttgtggcggcggtccagctccgattcggcgctgcaccaaagtgcgctggtggcgggcttcaatagcgacgtgaactcgatgggcgccaactatcagcagcagcaacatcagcaacaacagcaaccgggccagccaagatctcactcgccgcaccatggtataaacaggaccatgagtccgcaggcgcaacggaggaagtcgccgctactgcagccccatcagctgcagttgcagcaactgcaacagcagcagcaacagatgcaacatcagcatcagctgcaccagcagctccaaatgcagcagctgcaacagcaccagcagcaacaccagcagcagcagcaacaacagaacacgccatacaacaacgccaaattcacgaatcctgtgttccggccgctgcaggatcaggtcaactttgccaacaccggctccctgcccgatctcacggcccttcaaaactatggaccccagcagcagcagcagcaatcccagcaacagccgtcgcagcaacaacagcagttgcagcaaaccctgtcgccagtcatgtctccgcacaatcaccgccgcgaacgggatcagtcgcccagtccgtttagtccggcgggtggaggagggggagcaggtcccgggtcgccctatcagcagcaacagcactcgcccaccggaaacacgcaacagcagcagcagcagcaccaacagcccagcaactcgccgcacctgtcctttaccaatctggccaccacgcaggcagctgttaccacatttaacccgctccccacgctgggtccgcacaatgccaccgactaccgccagccaccgaatcctcctagtccacgctcttcgcccggcttgctgagcagcgtatcggccacggatctgcactccagtgcaccggccagtcccatacgccagcagcaacaggcccatcagcagcaacagcagcagcaacaggcgcagcaacaacagcaacagtttgataactcctacaacagtctgaatacctcgtttcacaatcagtttgagattttctcgctgggcgacagcaattcctcgccggaacagcagggctttgcaaataatttcgtggccctcgactttgacgacctgagtggcggcggaggtggtggcccaagcgggggcggcggcagcaatggaggaggtctgaccaacggttacaacaagccggagatgttggacttcagcgagctgagcggcagcccggaggcgagtgggaacaacaaccacatgcggcgaggagtgagcaacctgaacaacaacgggttgagcaatggtgtggtgggatccacgcacaacggcagcacaaatctaaatggagcgggaaacaacaatagcagtagtggaggtggcacggcgcaggatcctttgggaataaccacttcgcctgtgccctcacccttgggctgccccagttcaccgctgccgataccgattccgatgtcggcgcaaagctcgccacagcagcagcaccaccatcatcagcagcagcaacaacagcatcatcagcagcaacaccatcagcagcagcaattatcattatctctgcaccattcgccgcatcattcgccaatgcattcgccgcaccatgggaattcaccgctttcaagcagctcgccagtgagtcacaatgcctgctccaactccaacgtggtgatgaaccaccagcagcagcagcaacaacatcaccaccagcaacaccatcatcagggctcctcgcaaagtcacacgccgaccacagcgaatataccctctattatctttagtgattactcctccaacgcggattataccagggagatcttcgactccctcgatctggatctgggacagatggacgtagccggtttgcagatgctgtccgaccagaaccccatcatgatcgccgatcccaacatcgaggatagttttcgacgcgacctcaactgatactatgaggaggctgttgcggccattgagagcggagtgctgctggaggaggactaccaggcgctgctcggatcagaggcgctggcggatgaacaggtggtcacagtcgaggccgccggagccgcagcagcagtagtaacagttgaagaggcagccacagttagcgagaaggacaaaaaagatttggaagttgtggaacttctggtgtccggtgttatggatgacctggtggactccagtgacctggacgaggaagtgcgcaatttctttttttaggcagccagcaagtcatttttgtcgttaacacaactgatggaattttcgtttttaacacagatgaggaagtgaattacgttttttaaacgcattcacttgccatttctcgattaaatgccatattacttaagctcaggatttacaagcttaatgcgaattaagttaatttcggaaatgctgacgagagtgattgcaaagttcaaaattgatacaaattcacttccgcaaattcatgctgaaactgaaagttttctaacagtcctcaatattgttatctcgttatcgtccgtgctttcgtagctagctcctacaacaaaaatac

SEQ ID NO: 27 - protein, 797 aa, drosophila melanogaster
MANPRKFSEKIALQKQKQAEGTAEFERIMKEVYATKRDEPPANQKILDGLVGGQEVSQSSPGAGNGTGGGGSGSGSGASGGGASPDGLGGGGGSPTAYRESRGRSVGVGPMRRPSERKQDRSPYGSSSTQQTLDNGQLNPHLLGPPTAESLWRRSSSDSALHQSALVAGFNSDVNSMGANYQQQQHQQQQQPGQPRSHSPHHGINRTMSPQAQRRKSPLLQPHQLQLQQLQQQQQQMQHQHQLHQQLQMQQLQQHQQQHQQQQQQQNTPYNNAKFTNPVFRPLQDQVNFANTGSLPDLTALQNYGPQQQQQQSQQQPSQQQQQLQQTLSPVMSPHNHRRERDQSPSPFSPAGGGGGAGPGSPYQQQQHSPTGNTQQQQQQHQQPSNSPHLSFTNLATTQAAVTTFNPLPTLGPHNATDYRQPPNPPSPRSSPGLLSSVSATDLHSSAPASPIRQQQQAHQQQQQQQQAQQQQQQFDNSYNSLNTSFHNQFEIFSLGDSNSSPEQQGFANNFVALDFDDLSGGGGGGPSGGGGSNGGGLTNGYNKPEMLDFSELSGSPEASGNNNHMRRGVSNLNNNGLSNGVVGSTHNGSTNLNGAGNNNSSSGGGTAQDPLGITTSPVPSPLGCPSSPLPIPIPMSAQSSPQQQHHHHQQQQQQHHQQQHHQQQQLSLSLHHSPHHSPMHSPHHGNSPLSSSSPVSHNACSNSNVVMNHQQQQQQHHHQQHHHQGSSQSHTPTTANIPSIIFSDYSSNADYTREIFDSLDLDLGQMDVAGLQMLSDQNPIMIADPNIEDSFRRDLN

SEQ ID NO: 28 - DNA, 2416 bp, mouse
  misc_feature 1528: n = A,T,C or G
gggacgaagagtaggagtaggaggaggcggcgagaagatggcgacttcgaacaatccgcggaaatttagcgagaagatcgcactgcacaaccagaagcaggcggaggagacggcggccttcgaggaggtcatgaaggacctgagcctgacgcgggccgcgcggcttcagctgcagaagtcccagtacctgcagctgggccccagccgtggccagtactacggtgggtccctgcccaacgtgaaccagattggaagcagcagcgtggacctggccttccagaccccatttcagtcctcaggcctggacacgagtcggaccacacgacatcatgggcttgtggacagagtatatcgtgagcgtggcagacttggctccccgcaccgtcgacccctgtcagtagacaagcatgggcgacaggctgacagctgcccctatggcaccgtgtacctctcgcctcctgcggacaccagctggaggaggaccaactctgactctgccctgcaccagagcacaatgacacccagccaggcagagtccttcacaggcgggtcccaggatgcgcaccagaagagagtcttactgctaactgtcccaggaatggaggacaccggggctgagacagacaagaccctttctaagcagtcatgggactcaaagaaggcgggttccaggcccaagtcctgtgaggtccccggaatcaacatctttccgtctgcagaccaggagaacacaacagccctgatccctgccacccacaacacagggggctcccttcctgacctcaccaacatccacttcgcctccccactcccgacaccactggaccctgaggagcctccgttccctgctctcaccagctccagcagcaccggcagccttgcacatctgggcgttggcggcgcaggcggtatgaacacccccagctcttctccacagcaccggccagcagtcgtcagccccctgtccctgagcacagaggccaggcggcagcaggcccagcaggtgtcacccaccctgtctccgttgtcacccatcactcaggccgtggctatggatgccctgtccttggagcagcagctgccctatgccttcttcacccagactggctcccagcagcctcccccacagccccagccaccgcctccacctccaccggtatcccagcagcagccaccacctccacaggtgtctgtgggcctcccccagggtggtccactgctgcccagtgccagcctgactcgggggccccagctgccaccactctcagttactgtaccatccactcttccccagtcccctacagagaacccaggccagtcaccaatggggatcgatgccacttcggcaccagctctgcagtaccgcacgagtgcagggtcacctgccacccagtctcccacctctccggtctccaaccaaggcttctcccctggaagctccccacagcacacgtccaccctgggcagcgtgtttggggatgcgtactatgagcagcagatgacagccaggcaggccaatgctctgtcncgccagctggagcagttcaacatgatggagaacgccatcagctccagcagcctatacaacccgggctccacactcaactattcccaggctgccatgatgggtctgagcgggagccacgggggcctacaggacccgcagcagctcggctacacaggccacggtggaatccccaacatcatcctcacggtgacaggagagtcaccaccgagcctctctaaggaactgagcagcacactggcaggagtcagtgatgtcagctttgattcggaccatcagtttccactggacgagctgaagattgaccctctgaccctggacggactccatatgttgaatgacccagacatggttttagccgacccagccaccgaggacaccttccgaatggaccgcctgtgagtggctgtgcccaccagccgccgctggtcagtctccaacggcgctgccccaaacctggggacggcaatggcgtccccctttgccaacggccaagcttgtggttctgagcttgcaatgctgcccagtgcccctgccagccccccgccaccccggtcgttcacctcccatgatgcctggcgtgcgtgaggccgctgtgtactaggctggctatctgtctgtccatccatctacctggggtcaggctgatggccgaggctgtgagtgcctggcccccatggatgttccccgtgctcgctccctcacccctcactggggatgtgagagccctcatcagatacccaaagtgtcactcacttccagcatgtgctgtgcaacggagggccggggcgtgggtgtggagcgcccagaggcttaggtgcgccatccattcgactgttgtcagctgtcactgccttcctccatcctgtcccccgtcccaccgccatccct

SEQ ID NO: 29 - protein, 629 aa, mouse
MATSNNPRKFSEKIALHNQKQAEETAAFEEVMKDLSLTRAARLQLQKSQYLQLGPSRGQYYGGSLPNVNQIGSSSVDLAFQTPFQSSGLDTSRTTRHHGLVDRVYRERGRLGSPHRRPLSVDKHGRQADSCPYGTVYLSPPADTSWRRTNSDSALHQSTMTPSQAESFTGGSQDAHQKRVLLLTVPGMEDTGAETDKTLSKQSWDSKKAGSRPKSCEVPGINIFPSADQENTTALIPATHNTGGSLPDLTNIHFASPLPTPLDPEEPPFPALTSSSSTGSLAHLGVGGAGGMNTPSSSPQHRPAVVSPLSLSTEARRQQAQQVSPTLSPLSPITQAVAMDALSLEQQLPYAFFTQTGSQQPPPQPQPPPPPPPVSQQQPPPPQVSVGLPQGGPLLPSASLTRGPQLPPLSVTVPSTLPQSPTENPGQSPMGIDATSAPALQYRTSAGSPATQSPTSPVSNQGFSPGSSPQHTSTLGSVFGDAYYEQQMTARQANALSRQLEQFNMMENAISSSSLYNPGSTLNYSQAAMMGLSGSHGGLQDPQQLGYTGHGGIPNIILTVTGESPPSLSKELSSTLAGVSDVSFDSDHQFPLDELKIDPLTLDGLHMLNDPDMVLADPATEDTFRMDRL

SEQ ID NO: 30 - protein, 566 aa, fugu rubripres
MASSNNPRKFSEKIALHNQKQAEETAAFEEVMKDLNVTRAARLQLQKTQYLQLGQNRGQYYGGSLPNVNQIGNGNIDLPFQVSNSVLDTSRTTRHHGLVERVYRDRNRISSPHRRPLSVDKHGRQRTNSDSALHQSAMNPKPHEVFAGGSQELQPKRLLLTVPGTEKSESNADKDSQEQSWDDKKSIFPSPDQELNPSVLPAAHNTGGSLPDLTNIQFPPPLSTPLDPEDTVTFPSLSSSNSTGSLTTNLTHLGISVASHGNNGEKNIFFLKTCTSCEDVYDFYFVGIPTSSQTTMTATAQRRQPPVVPLTLTSDLTLQQSPQQLSPTLSSPINITQSMKLSASSLQQYRNQTGSPATQSPTSPVSNQGFSPGSSPQPQHIPVVGSIFGDSFYDQQLALRQTNALSHQVCEDGRRLEITHVRLSRLHAELCFCFSQLEQFNMIENPISSTSLYNQCSTLNYTQAAMMGLTGSSLQDSQQLGYGNHGNIPNIILTISVTGESPPSLSKELTNSLAGVGDVSFDPDTQFPLDELKIDPLTLDGLHMLNDPDMVLADPATEDTFRMDRL

SEQ ID NO: 31 - DNA, 1602 bp, fugu rubripres
atggcgtcctctaacaatcctcgcaaatttagcgaaaaaatcgcactgcataaccagaaacaagcagaggagactgctgcgttcgaagaagtgatgaaggacctgaacgtcacaagggctgcccgggtaagacagctgcagttacagaagacccagtatttgcaactagggcagaatcgtggacagtactatggaggctcactgcccaatgtcaatcagattggaaatggcaacattgacctgccttttcaggtgagcaggacaaactcagactcagctttacatcagagtgccatgaatccaaagccccacgaagtgtttgctggggggtcgcaggagctgcagcccaaacgactgctgctaacagtgcctggaaccgaaaaatcggaatcaaacgcagacaaagattcgcaggagcagtcgtgggatgacaaaaagagtatttttccatcaccagaccaggagttaaacccctccgtgcttccagccgcgcacaacaccggcggttcgctccccgacctgaccaacatccagttccctcctccactgtccaccccactggaccccgaggacaccgtcaccttcccctccctcagctcctctaacagcacaggcagtctgactaccaacctcacccacctgggcatcagtgtggccagccatggtaataacggagagaaaaatatattttttttaaaaacatgcacttcatgcgaggatgttaaataatattacgacttttattttgtagggattcccacttcctctcaaaccaccatgacagcaacagcacagcggcggcaaccacccgtggtccccctcaccctcacctctgacctgactcttcaacagtccccccagcagctttcacccaccctctcctcacccattaacatcacacagagcatgaagcttagtgctagctaacattcttccctccaacagtaccgcaatcagactggctcaccagccactcagtctccaacctccccagtctccaatcaaggcttctcccccggcagctcgcctcaaccacagcacattcctgtggtgggcagtatatttggggactccttctatgatcagcagttggctctgaggcagaccaatgccctttctcatcaggtgtgtgaggacggccgcaggttagaaataacacacgtacgtctctcacgacttcacgccgagctttgtttttgtttttctcagctggagcagttcaatatgatagagaaccccatcagctccaccagcctgtacaatcagtgctccacccttaattacacacaggcagccatgatgggcctcaccgggagcagcctgcaggactcgcagcagctcggctacggcaatcacggcaacatccccaacatcatactgacaatttcagtcacaggggagtctccgccgagcctctccaaagagctgaccaactcattggccggcgtcggcgacgtcagctttgatccagacacgcagtttcctctggacgagctgaagatcgacccgctgaccttggacggcctgcacatgctcaacgacccagacatggtgctggcagaccccgccacagaggacacgttcaggatggacaggctgtaa

SEQ ID NO: 32 - protein, 170 aa, human
MATSNNPRKFSEKIALHNQKQAEETAAFEEVMKDLSLTRAARLQLQKSQYLQLGPSRGQYYGGSLPNVNQIGSGTMDLPFQPSGFLGEALAAAPVSLTPFQSSGLDTSRTTRHHGLVDRVYRERGRLGSPHRRPLSVDKHGRQADSCPYGTMYLSPPADTSWRRTNSDSA

SEQ ID NO: 33 - protein, 356 aa, human
MATSNNPRKFSEKIALHNQKQAEETAAFEEVMKDLSLTRAARLQLQKSQYLQLGPSRGQYYGGSLPNVNQIGSGTMDLPFQPSGFLGEALAAAPVSLTPFQSSGLDTSRTTRHHGLVDRVYRERGRLGSPHRRPLSVDKHGRQADSCPYGTMYLSPPADTSWRRTNSDSALHQSTMTPTQPESFSSGSQDVHQKRVLLLTVPGMEETTSEADKNLSKQAWDTKKTGSRPKSCEVPGINIFPSADQENTTALIPATHNTGGSLPDLTNIHFPSPLPTPLDPEEPTFPALSSSSSTGNLAANLTHLGIGGAGQGMSTPGSSPQHRPAGVSPLSLSTEARRQQASPTLSPLSPITQAVA

SEQ ID NO: 34 - protein, 494 aa, human
MATSNNPRKFSEKIALHNQKQAEETAAFEEVMKDLSLTRAARLQLQKSQYLQLGPSRGQYYGGSLPNVNQIGSGTMDLPFQPSGFLGEALAAAPVSLTPFQSSGLDTSRTTRHHGLVDRVYRERGRLGSPHRRPLSVDKHGRQADSCPYGTMYLSPPADTSWRRTNSDSALHQSTMTPTQPESFSSGSQDVHQKRVLLLTVPGMEETTSEADKNLSKQAWDTKKTGSRPKSCEVPGINIFPSADQENTTALIPATHNTGGSLPDLTNIHFPSPLPTPLDPEEPTFPALSSSSSTGNLAANLTHLGIGGAGQGMSTPGSSPQHRPAGVSPLSLSTEARRQQASPTLSPLSPITQAVAMDALSLEQQLPYAFFTQAGSQQPPPQPQPPPPPPPASQQPPPPPPPQAPVRLPPGGPLLPSASLTRGPQPPPLAVTVPSSLPQSPPENPGQPSMGIDIASAPALQQYRTSAGSPANQSPTSPVSNQGFSPGSSPQHTS

SEQ ID NO: 35 - protein, 580 aa, human
MATSNNPRKFSEKIALHNQKQAEETAAFEEVMKDLSLTRAARLQLQKSQYLQLGPSRGQYYGGSLPNVNQIGSGTMDLPFQPSGFLGEALAAAPVSLTPFQSSGLDTSRTTRHHGLVDRVYRERGRLGSPHRRPLSVDKHGRQADSCPYGTMYLSPPADTSWRRTNSDSALHQSTMTPTQPESFSSGSQDVHQKRVLLLTVPGMEETTSEADKNLSKQAWDTKKTGSRPKSCEVPGINIFPSADQENTTALIPATHNTGGSLPDLTNIHFPSPLPTPLDPEEPTFPALSSSSSTGNLAANLTHLGIGGAGQGMSTPGSSPQHRPAGVSPLSLSTEARRQQASPTLSPLSPITQAVAMDALSLEQQLPYAFFTQAGSQQPPPQPQPPPPPPPASQQPPPPPPPQAPVRLPPGGPLLPSASLTRGPQPPPLAVTVPSSLPQSPPENPGQPSMGIDIASAPALQQYRTSAGSPANQSPTSPVSNQGFSPGSSPQHTSTLGSVFGDAYYEQQMAARQANALSHQLEQFNMMENAISSSSLYSPGSTLNYSQAAMMGLTGSHGSLPDSQQLGYASHSGIPNIILT

SEQ ID NO: 36 - protein, 481 aa, human
ALHQSTMTPTQPESFSSGSQDVHQKRVLLLTVPGMEETTSEADKNLSKQAWDTKKTGSRPKSCEVPGINIFPSADQENTTALIPATHNTGGSLPDLTNIHFPSPLPTPLDPEEPTFPALSSSSSTGNLAANLTHLGIGGAGQGMSTPGSSPQHRPAGVSPLSLSTEARRQQASPTLSPLSPITQAVAMDALSLEQQLPYAFFTQAGSQQPPPQPQPPPPPPPASQQPPPPPPPQAPVRLPPGGPLLPSASLTRGPQPPPLAVTVPSSLPQSPPENPGQPSMGIDIASAPALQQYRTSAGSPANQSPTSPVSNQGFSPGSSPQHTSTLGSVFGDAYYEQQMAARQANALSHQLEQFNMMENAISSSSLYSPGSTLNYSQAAMMGLTGSHGSLPDSQQLGYASHSGIPNIILTVTGESPPSLSKELTSSLAGVGDVSFDSDSQFPLDELKIDPLTLDGLHMLNDPDMVLADPATEDTFRMDRL

SEQ ID NO: 37 - DNA, 30 bp, Artificial Sequence
  primer
caacatggccaatccgcgcaagttcagcga

SEQ ID NO: 38 - DNA, 29 bp, Artificial Sequence
  primer
tcagttgaggtcgcgtcgaaaactatcct

SEQ ID NO: 39 - DNA, 62 bp, drosophila melanogaster
ggagcctggcgtcagagagcctggcgtcagagagcctggcgtcagagagcctggcgtcagag

(19) United States
(12) Patent Application Publication
(10) Pub. No.: US 2007/0036810 A1
(43) Pub. Date: Feb. 15, 2007

(54) Cyclic amp response element activator proteins and uses related thereto

(76) Inventors: Vadim Iourgenko, Sharon, MA (US); Mark Aron Labow, Lexington, MA (US); Chuanzheng Song, Southborough, MA (US); Wenjun Zhang, Rockville, MD (US); Jian Zhu, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/549,977

(22) PCT Filed: Mar. 25, 2004
(86) PCT No.: PCT/EP04/03182

§ 371 Date: Jun. 28, 2006

Related U.S. Application Data
(60) Provisional application No. 60/457,695, filed on Mar. 26, 2003.
(60) Provisional application No. 60/463,934, filed on Apr. 18, 2003.

(51) Int. Cl. C12Q 1/68; C07H 21/04; C12P 21/06; A61K 39/00; C07K 14/705

(52) U.S. Cl. 424/185.1; 435/6; 435/69.1; 435/320.1; 435/325; 530/350; 530/388.22; 536/23.5

(57) ABSTRACT

The invention discloses newly identified cyclic AMP response element activator proteins (CREAP proteins). It is contemplated herein that said proteins are suitable drug targets for the development of new therapeutics to prevent, treat or ameliorate pathological conditions related to abnormal activation of CRE-dependent gene expression or abnormal activation of chemokines. The invention relates to methods to prevent, treat or ameliorate said pathological conditions and pharmaceutical compositions therefor comprising modulators with inhibitory effect on CREAP protein activity and/or CREAP gene expression. The invention also relates to methods to identify compounds with therapeutic usefulness to treat said pathological conditions, comprising identifying compounds that can inhibit CREAP protein activity and/or CREAP gene expression.

### BACKGROUND OF THE INVENTION

Cyclic-AMP response element binding protein (CREB), activation transcription factor 1 (ATF1) and cAMP response element modulator (CREM) are a subgroup of closely related proteins belonging to the basic-region leucine zipper (bZIP) transcription factor superfamily. They are the central mediators of transcriptional control exerted by a variety of extracellular stimuli such as hormones, growth factors, neuropeptides and neurotransmitters, calcium, hypoxia and oxidative stress. It is well established, mostly through studies of CREB, that phosphorylation of conserved serine residues in the kinase-inducible domain (KID) of these proteins lead to transcriptional activation of a spectrum of target genes involved in cell growth regulation and differentiation, metabolism, reproduction and development, neuronal activity modulation and immune regulation. All these target genes share a conserved cis-acting cyclic-AMP response element (CRE), which has the palindromic sequence of TGACGTCA or asymmetric variations which include a CRE half site with the core sequence TGAC (see Mayr B, Montminy M., Nat Rev Mol Cell Biol 2001 August;2(8):599-609).

Transcription regulation occurs when phosphorylated CREB/CREM/ATF1 homo- and/or heterodimers bind to the CRE site through the bZIP domains, while the KID domains recruit effector molecules such as the 265 kD CREB binding protein CBP or p300 and associated Pol II basal transcription machinery to the proximity of the transcription start site.

A tremendous amount of research has been devoted to identify molecules linking cell stimulation to activation of CREB/CREM/ATF1. The complexity of these activators is exemplified by the study of kinases for CREB phosphorylation. Originally, CREB was considered an exclusive transcription mediator to extracellular stimuli that increase cAMP, which in turn activates protein kinase A (PKA) for CREB phosphorylation. However, subsequent investigations revealed that CREB proteins are also phosphorylated by pp90RSK in response to growth factors, MSK-1 in response to mitogens and stress, CAMK II/IV in response to Ca⁺⁺ elevation and AKT in response to hypoxia and survival signals. It is apparent from these studies that regulation of the activities of the CREB/CRE/ATF1 family proteins is extremely complex to ensure specificity and sensitivity, in a cell context-dependent manner, in generating appropriate cellular output from a wide array of extracellular stimuli.

We describe herein results of a genome-scale cell based functional screening of a large collection of full-length human cDNA clones, representing transcripts from 11,000 to 15,000 genes, for proteins that activate CRE-dependent gene expression. Data indicate several heretofore unidentified CRE activators, including KIAA0616, a gene of previously unknown function and which has been renamed herein CREAP1. Applicants have also discovered two more distinct human proteins similar in structure and activity to CREAP1, termed herein CREAP2 and CREAP3, as well as mouse and Drosophila homologs, all of which are members of a heretofore unknown family of genes that regulate CRE-dependent gene expression.

Applicants also report herein the surprising discovery that CREAP1 is a potent inducer of other proteins including phosphoenolpyrovate carboxy kinase (PEPCK), amphiregulin and chemokines such as IL-8 and Exodus-1/MIPalpha. As such, it is contemplated herein that the CREAP proteins of the present invention can be used as novel drug targets for the treatment of pathological conditions related to the abnormal activation of genes that contain CRE site(s) in their promoter regions as well as for the treatment of conditions associated with abnormal activation of PEPCK, amphiregulin and chemokines, particularly IL-8 and Exodus-1/MIPalpha. These conditions include, but are not limited to, osteoarthritis, psoriasis, asthma, COPD, rheumatoid arthritis, cancer, pathological angiogenesis, diabetes, hypertension, chronic pain and other inflammatory and autoimmune diseases as well as neurodegenerative conditions such as Alzheimer's Disease, Parkinson's Disease and Huntington Disease.

The invention also provides a method for identifying modulators that inhibit or enhance CREAP activity and/or inhibit or enhance CREAP gene expression and the use of such modulators for the treatment of these conditions in human and veterinary patients. The invention also provides pharmaceutical compositions comprising said modulators.

### SUMMARY OF THE INVENTION

The instant application relates to the discovery of a new family of proteins, referred to herein as CREAP, which are activators of CRE-dependent transcription as well as inducers of chemokines. As such, it is contemplated herein that members of this family of proteins are suitable targets for the development of new therapeutics to prevent, treat or ameliorate pathological conditions related to abnormal activation of CRE-dependent gene expression or abnormal activation of chemokines including, but not limited to, osteoarthritis, psoriasis, asthma, COPD, rheumatoid arthritis, cancer, pathological angiogenesis, diabetes, hypertension, chronic pain, and other inflammatory and autoimmune diseases. In addition, as loss of CREB function has been associated with deficits in learning and neurodegeneration, agonists of CREAP proteins may be useful to prevent, treat or ameliorate neurodegenerative disorders such as Alzheimer's , Parkinson's and Huntington diseases. Thus, in one aspect the invention relates to a method to identify modulators useful to prevent, treat or ameliorate said conditions, comprising: a) assaying for the ability of a candidate modulator, in vitro, ex vivo or in vivo, to inhibit or enhance the activity of a CREAP protein and/or inhibit or enhance the expression of a CREAP protein and which can further include b) assaying for the ability of an identified CREAP modulator to reverse the pathological effects observed in in vivo, ex vivo or in vitro models of said pathological conditions and/or in clinical studies with subjects with said pathological conditions.

In another aspect, the invention relates to a method to prevent, treat or ameliorate pathological conditions related to abnormal activation of CRE-dependent gene expression or abnormal activation of chemokines, comprising administering to a subject in need thereof an effective amount of a CREAP modulator, wherein said modulator, e.g., inhibits or enhances the activity of any one or more of said CREAP proteins or Inhibits or enhances the expression of any one or more CREAP proteins wherein said CREAP protein is selected from the group consisting of CREAP1, CREAP2 and CREAP3.

In one embodiment, the modulator comprises any one or more substances selected from the group consisting of antisense oligonucleotides, triple helix DNA, ribozymes, RNA aptamers, siRNA and double or single stranded RNA wherein said substances are designed to inhibit the expression of a CREAP protein. In a further embodiment, the modulator comprises antibodies to a CREAP protein or fragments thereof, wherein said antibodies or fragment thereof can inhibit the activity of said CREAP protein. In a further embodiment of this invention, the modulator comprises peptide mimetics of a CREAP protein wherein said peptide mimetic can inhibit the activity of said CREAP protein. It is contemplated herein that one or more modulators described herein may be administered concurrently.

In another aspect, the invention relates to a method to treat, prevent or ameliorate pathological conditions related to abnormal activation of CRE-dependent gene expression or abnormal activation of chemokines comprising administering to a subject in need thereof a pharmaceutical composition comprising an effective amount of a CREAP modulator. In one embodiment, said modulator inhibits or enhances the activity of a CREAP protein or inhibits or enhances the expression of a gene encoding said protein in a subject wherein said CREAP protein is selected from the group consisting of CREAP1, CREAP2 or CREAP3. In one embodiment, the modulator comprises any one or more substances selected from the group consisting of antisense oligonucleotides, triple helix DNA, ribozymes, RNA aptamers, siRNA and double or single stranded RNA wherein said substances are designed to inhibit expression of a CREAP protein. In a further embodiment, the modulator comprises antibodies or peptide mimetics to a CREAP protein or fragments thereof, wherein said antibodies or mimetics can e.g., inhibit enzymatic or other activity of said CREAP protein. It is contemplated herein that one or more modulators of one or more of said proteins may be administered concurrently.

In another aspect, the invention relates to a pharmaceutical composition comprising one or more CREAP modulators in an amount effective to treat, prevent or ameliorate pathological conditions related to abnormal activation of CRE-dependent gene expression or abnormal activation of chemokines in a subject in need thereof wherein said modulator can inhibit or enhance the activity of a CREAP protein and/or inhibit or enhance the expression of a CREAP protein wherein said CREAP protein is selected from the group consisting of CREAP1, CREAP2 or CREAP3. In a further embodiment, the modulator comprises any one or more substances selected from the group consisting of antisense oligonucleotides, triple helix DNA, ribozymes, RNA aptamers, si RNA and double or single stranded RNA wherein said substances are designed to inhibit CREAP expression. In a further embodiment, the modulator comprises antibodies to or peptide mimetics of a CREAP protein or fragments thereof, wherein said antibodies or mimetics can e.g., inhibit enzymatic or other activity of said CREAP protein.

In another aspect, the invention relates to a pharmaceutical composition comprising CREAP proteins.

In yet another aspect, the invention relates to a method to treat, prevent or ameliorate pathological conditions related to abnormal activation of CRE-dependent gene expression or abnormal activation of chemokines comprising administering to a subject in need thereof a pharmaceutical composition comprising CREAP proteins.

In another aspect, the invention relates to a method to diagnose subjects suffering from a pathological condition related to abnormal activation of CRE-dependent gene expression or abnormal activation of chemokines who may be suitable candidates for treatment with CREAP modulators or exogenous CREAP proteins comprising detecting levels of CREAP protein in a biological sample from said subject wherein subjects with abnormal levels compared to controls would be a suitable candidate for treatment.

In yet another aspect, the invention relates to a method to diagnose a subject suffering from a pathological condition related to abnormal activation of CRE-dependent gene expression or abnormal activation of chemokines who may be a suitable candidate for treatment with one or more CREAP modulators or exogenous CREAP proteins comprising assaying mRNA levels of CREAP protein in a biological sample from said subject wherein a subject with abnormal mRNA levels compared to controls would be a suitable candidate for treatment.

In yet another aspect, there is provided a method to treat, prevent or ameliorate a pathological condition related to abnormal activation of CRE-dependent gene expression or abnormal activation of chemokines comprising: (a) assaying for CREAP mRNA and/or CREAP protein levels in a subject and (b) administering to a subject with abnormal levels of mRNA and/or CREAP protein compared to controls a CREAP modulator or exogenous CREAP proteins in an amount sufficient to treat, prevent or ameliorate said pathological condition.

In yet another aspect of the present invention there are provided assay methods and kits comprising the components necessary to detect expression of polynucleotides encoding CREAP proteins or levels of CREAP proteins or fragments thereof, in biological samples derived from a patient, said kits comprising, e.g., antibodies or peptide mimetics that bind to CREAP proteins, or to fragments thereof, or polynucleotide probes that hybridize with CREAP polynucleotides. In a preferred embodiment, such kits also comprise instructions detailing the procedures by which the kit components are to be used.

The present invention also pertains to the use of a CREAP modulator or exogenous CREAP proteins in the manufacture of a medicament for the treatment, prevention or amelioration of pathological conditions related to abnormal activation of CRE-dependent gene expression or abnormal activation of chemokines. Preferably, said pathological condition is an autoimmune or neurodegenerative disease. In one embodiment, said modulator comprises any one or more substances selected from the group consisting of antisense oligonucleotides, triple helix DNA, ribozymes, RNA aptamer, siRNA and double or single stranded RNA wherein said substances are designed to inhibit CREAP gene expression. In yet a further embodiment, said modulator comprises one or more antibodies to a CREAP protein or fragments thereof, wherein said antibodies or fragments thereof can, e.g., inhibit enzymatic or other CREAP activity. In another embodiment, said modulator comprises one or more peptide mimetics of a CREAP protein, wherein said mimic can e.g. inhibit enzymatic or other CREAP activity.

The invention also pertains to exogenous CREAP proteins or modulators of CREAP proteins for use as a pharmaceutical. In one embodiment, said modulator comprises any one or more substances selected from the group consisting of antisense oligonucleotides, triple helix DNA, ribozymes, RNA aptamer, siRNA and double or single stranded RNA wherein said substances are designed to inhibit CREAP expression. In yet a further embodiment, said modulator comprises one or more antibodies to or peptide mimetics of CREAP, or fragments thereof, wherein said antibodies, mimetics or fragments thereof can, e.g., inhibit enzymatic or other CREAP activity. In another embodiment, said modulator comprises one or more peptide mimetics of a CREAP protein, wherein said mimetic can e.g. inhibit enzymatic or other CREAP activity.

As the correct polynucleotide sequence of CREAP2 and CREAP 3 have heretofore not been disclosed, it is contemplated herein that the present invention also provides isolated polypeptides comprising amino acid sequences set forth in SEQ ID NO:16 and SEQ ID NO:25, respectively. Furthermore, the invention provides isolated polypeptides consisting of amino acid sequences set forth in SEQ ID NO:16 and SEQ ID NO:25 and fragments thereof. In accordance with this aspect of the invention there are provided novel polypeptides of human origin as well as biologically, diagnostically or therapeutically useful fragments, variants, homologs and derivatives thereof, variants and derivatives of the fragments, and analogs of the foregoing.

The present invention also makes available isolated nucleic acids comprising nucleotide sequences encoding the CREAP proteins disclosed herein, particularly, CREAP2 and CREAP3 and homologs and fragments thereof and/or equivalents or nucleic acids that are substantially similar to the nucleic acids with the nucleotide sequences as sent forth in SEQ ID NO 15 and SEQ ID NO:24. In a preferred embodiment, the isolated DNA takes the form of a vector molecule comprising at least a fragment of a DNA of the present invention, in particular comprising the DNA consisting of a nucleotide sequence as set forth in SEQ ID NO:1, SEQ ID NO.15 or SEQ ID NO:24.

Another aspect of the invention provides a process for producing the aforementioned polypeptides, polypeptide fragments, variants and derivatives, fragments of the variants and derivatives, and analogs of the foregoing. In a preferred embodiment of this aspect of the invention there are provided methods for producing the aforementioned CREAP proteins comprising culturing host cells having incorporated therein an expression vector containing an exogenously-derived nucleotide sequence encoding such a polynucleotide under conditions sufficient for expression of the polypeptide in the host cell, thereby causing expression of the polypeptide, and optionally recovering the expressed polypeptide.

In a preferred embodiment of this aspect of the present invention, there is provided a method for producing polypeptides comprising or consisting of an amino acid sequence as set forth in SEQ ID NO:2, SEQ ID NO:16, or SEQ ID NO:25, which comprises culturing a host cell having incorporated therein an expression vector containing an exogenously-derived polynucleotide encoding a polypeptide comprising or consisting of an amino acid sequence as set forth in SEQ ID NO:2, SEQ ID NO:16, SEQ ID NO:25, under conditions sufficient for expression of such a polypeptide in the host cell, thereby causing the production of an expressed polypeptide, and optionally recovering the expressed polypeptide. Preferably, in any of such methods the exogenously derived polynucleotide comprises or consists of the nucleotide sequence set forth in SEQ ID NO:1, the nucleotide sequence set forth in SEQ ID NO:15, or the nucleotide sequence set forth in SEQ ID NO:24. In accordance with another aspect of the invention there are provided products, compositions, processes and methods that utilize the aforementioned polypeptides and polynucleotides for, inter alia, research, biological, clinical and therapeutic purposes.

In yet another aspect, the invention provides host cells which can be propagated in vitro, preferably vertebrate cells, in particular mammalian cells, or bacterial cells, which are capable upon growth in culture of producing a polypeptide that comprises the amino acid sequence set forth in SEQ ID NO:2, SEQ ID NO:16, SEQ ID NO:25, or fragments thereof, where the cells contain transcriptional control DNA sequences, preferably other than human CREAP transcriptional control sequences, where the transcriptional control sequences control transcription of DNA encoding a polypeptide with the amino acid sequence according to SEQ ID NO:2, SEQ ID NO:16, SEQ ID NO:25, or fragments thereof, including but not limited to amino acid sequences comprising the active portions and fragments of the CREAP proteins.

In yet another aspect, the invention is directed to methods for the introduction of nucleic acids of the invention into one or more tissues of a subject in need of treatment with the result that one or more proteins encoded by the nucleic acids are expressed and or secreted by cells within the tissue.

### DESCRIPTION OF THE FIGURES

FIG. 1 illustrates that CREAP1 is a highly conserved protein and contains a potent transcription activation domain. Amino acid sequence of human CREAP1 and the predicted murine, fugu and drosophila CREAP1 related genes are shown. Identical and highly conserved amino acids are shaded. A conserved potential PKA phosphorylation site is boxed. The first sequence represents human, second is mouse, third is Fugu and fourth is Drosophila.

FIG. 2 illustrates amino acid sequences of full length cDNAs corresponding to human and Drosophila CREAP proteins. Amino acids are aligned using ClustalW and conserved amino acids are shaded.

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Abbreviations:

ABIN-2 A20-Binding Inhibitor of NF-kappaB activation-2
ACT1 NFkB-activating protein 1
ANKRD3 Ankyrin Repeat Domain protein 3
AP-1 Activator Protein 1
ARHGEF1 Rho Guanine Nucleotide Exchange Factor (GEF) 1
ATCC American Type Culture Collection
ATF Activation transcription factor
BZIP Basic-region leucine zipper
C/EBP CCAAT/Enhancer Binding Protein
CAD Constitutive active domain
CAMK Ca⁺⁺/Calmodulin dependent protein kinase
cAMP Cyclic AMP
CBP CREB binding protein
CNS Central nervous system
COPD Chronic obstructive pulmonary disease
CR53 putative transcription factor CR53
CRE Cyclic AMP Response Element
CREB cyclic AMP Response Element Binding Protein
CREB1 cAMP Responsive Element Binding Protein 1
CRE-BPa cAMP response element-binding protein
CREM cAMP response element modulator
ERK Extracellular signal-regulated kinase
EST Expressed sequence tag
HPH2 human Homolog of Drosophila protein Polyhomeotic (Ph)
HPH2 Human Polycomb Homolog 2
HTS High-throughput Screening
IBMX 3-isobutyl-1-methylxanthine
ICER Inducible cAMP early repressor
IkBα Inhibitor of nuclear factor kappa-B kinase alpha subunit
IKK IkBα kinase
IKKγ IkBα kinase gamma
IL-1 Interleukin-1
IL-8 Interleukin-8
IL-8ₚ-Luc IL-8 Promoter-Reporter Driving Luciferase expression
IL-24 Interleukin-24
KIAA0616 hypothetical protein predicted by cDNA clone KIAA0616
KID Kinase inducible domain
MAP3K11 Mitogen-Activated Protein Kinase Kinase Kinase 11
MAP3K12 Mitogen-Activated Protein Kinase Kinase Kinase 12
MEK Mitogen-Activated Protein Kinase/ERK Kinase
MEKK Mitogen-activated protein kinase/ERK kinase kinase-1
MSK Mitogen and stress-activated protein kinase
NFAT nuclear factor of activated T cells
NF-IL-6 Nuclear factor-interleukin-6 transcription factor
NF-κB Nuclear Factor of kappa light polypeptide gene enhancer in B-cells
NPY Neuropeptide Y
NR2F2 Nuclear Receptor subfamily 2, group F, member 2
Oct-1 Octamer-binding transcription factor 1
Oct-1/C/EBP Octamer-binding transcription factor 1/CCAAT/Enhancer Binding
Protein
PCK1 Phosphoenolpyruvate Carboxy Kinase I
PKA Cyclic AMP-dependent protein kinase
POL II RNA polymerase II
relA Reficuloendotheliosis viral oncogene homolog A, alias NF-κB subunit 3, p65
Rho-GEF- p114 Rho-specific Guanine nucleotide Exchange Factor p114
RIPK2 Receptor-interacting serine-threonine kinase 2
RLU Relative Luminescence Unit
RSK Ribosomal S6 kinase
TBP TATA-binding protein
TEF1 Thyrofrophic Embryonic Factor 1
TF Transcriptional factor
TNFα Tumor necrosis factor-α
TRAF6 TNF receptor-associated factor 6
TSHα thyroid-stimulating hormone alpha
VCAM1 Vascular Cell Adhesion Molecule-1
XboxP X-box binding protein 1

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art. In addition, reference to a CREAP protein or “CREAP”, unless otherwise noted, includes any one or more of the CREAP proteins disclosed herein, particularly, any one or more of the human CREAP1-3 polypeptides identified herein as belonging to the CREAP family of proteins.

The ability of a substance to “modulate” a CREAP protein (e.g. a “CREAP modulator) includes, but is not limited to, the ability of a substance to inhibit or enhance the activity of a CREAP protein and/or inhibit or enhance the expression of any one or more of said proteins. Such modulators include both agonists and antagonists of CREAP activity. Such modulation could also involve effecting the ability of other proteins to interact with CREAP proteins, for example related regulatory proteins or proteins that are modified by CREAP.

The term “agonist”, as used herein, refers to a molecule (i.e. modulator) which, directly or indirectly, may modulate a polypeptide (e.g. a CREAP polypeptide) and which increase the biological activity of said polypeptide. Agonists may include proteins, nucleic acids, carbohydrates, or other molecules. A modulator that enhances gene transcription or the biochemical function of a protein is something that increases transcription or stimulates the biochemical properties or activity of said protein, respectively.

The terms “antagonist” or “inhibitor” as used herein, refer to a molecule (i.e. modulator) which directly or indirectly may modulate a polypeptide (e.g. a CREAP polypeptide) which blocks or inhibits the biological activity of said polypeptide. Antagonists and inhibitors may include proteins, nucleic acids, carbohydrates, or other molecules. A modulator that inhibits expression or the biochemical function of a protein is something that reduces gene expression or biological activity of said protein, respectively.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative ” is sometimes used in reference to the antisense strand, and “positive” is sometimes used in reference to the sense strand.

As contemplated herein, antisense oligonucleotides, triple helix DNA, RNA aptamers, siRNA, ribozymes and double or single stranded RNA are designed to inhibit CREAP expression such that the chosen nucleotide sequence of the protein to which the inhibitory molecule is designed can cause specific inhibition of endogenous CREAP production. For example, knowledge of the CREAP1 nucleotide sequence may be used to design an antisense molecule which gives strongest hybridization to CREAP mRNA without undue experimentation. Similarly, ribozymes can be synthesized to recognize specific nucleotide sequences of a protein of interest and cleave it (Cech. J. Amer. Med Assn. 260:3030 (1988)). Techniques for the design of such molecules for use in targeted inhibition of gene expression are well known to one of skill in the art.

The CREAP proteins disclosed herein include, but are not limited to, the human CREAP 1, CREAP2 and CREAP3 polypeptides, any and all forms of these polypeptides including, but not limited to, partial forms, homologs, isoforms, precursor forms, the full length polypeptides, fusion proteins containing the protein sequence or fragments of any of the above, from humans or any other species. Fragments of interest include, but are not limited to, those fragments containing amino acids of particular importance for normal CREAP function, including for example, amino acids 356-580. The sequence of CREAP1, and its variants, may be found in Genbank, Accession Numbers NM_{—}025021 and AB014516. The complete, correct sequences of CREAP2 and CREAP3, to the Applicant's knowledge, have not been previously disclosed; partial sequences may be found in Genbank (CREAP 2 Accession number XM_{—}117201 (DNA) and XP_{—}117201 (protein) and CREAP3 Accession number AK090443 (DNA) and BAC03424 (protein)). Homologs of CREAP include those disclosed herein, and those which would be apparent to one of skill in the art, and are meant to be included within the scope of the invention. It is also contemplated that CREAP proteins include those isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood In the art.

The term “sample” or “biological sample” as used herein, is used in its broadest sense. A biological sample from a subject may comprise blood, urine or other biological material with which activity or gene expression of CREAP proteins may be assayed.

As used herein, the term “antibody” refers to intact molecules as well as fragments thereof, such as Fa, F(ab′)₂, and Fv, which are capable of binding the epitopic determinant. Antibodies that bind the CREAP polypeptides disclosed herein can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A peptide mimetic is a synthetically derived peptide or non-peptide agent created based on a knowledge of the critical residues of a subject polypeptide which can mimic normal polypeptide function. Peptide mimetics can disrupt binding of a polypeptide to its receptor or to other proteins and thus interfere with the normal function of a polypeptide. For example, a CREAP mimetic would interfere with normal CREAP function.

A “therapeutically effective amount” is the amount of drug sufficient to treat, prevent or ameliorate pathological conditions related to abnormal activation of CRE-dependent gene expression or abnormal activation of chemokines.

“Related regulatory proteins” and “related regulatory polypeptides” as used herein refer to polypeptides involved in the regulation of CREAP proteins which may be identified by one of skill in the art using conventional methods such as described herein.

“Pathological conditions related to the abnormal activation of CRE-dependent gene expression or abnormal activation of chemokines” include, but is not limited to conditions such as: osteoarthritis, COPD, psoriasis, asthma, rheumatoid arthritis, cancer, pathological angiogenesis, diabetes, hypertension, chronic pain, and other inflammatory and autoimmune diseases as well as neurodegenerative conditions such as Alzheimer's Disease, Parkinson's Disease and Huntington Disease. Abnormal activation can include excessive activation, e.g., states where the mRNA encoding a CREAP protein is up-regulated or the protein products of these genes have enhanced activity in a cell through either increases in absolute quantity or specific activity as well as states in which there is a down-regulation of CRE-dependent gene expression or there is abnormally low chemokine activation.

As contemplated herein, the instant invention includes a method to use the CREAP genes and gene products disclosed herein to discover agonists and antagonists that induce or repress, respectively, CRE-dependent genes. As used herein, a “CRE-dependent” gene includes those genes that are dependent on a cyclic amp response element which acts through a CRE-binding protein such as CREB1, CREB2, CRE-BPa (for review, see Lonze, B., and Ginty, D. (2002) Neuron 35, 605; Muller FU, Neumann J, Schmitz W., Mol Cell Biochem 2000 September;212(1-2):11-7 and Mayr B, Montminy M. Nat Rev Mol Cell Biol 2001 August;2(8):599-609). These genes include, but are not limited to, genes that are vital to metabolic control such as PEPCK, Uncoupling protein-1, neuroregulatory molecules such as Galanin and tyrosine hydorxylase, and growth factors including insulin and amphiregulin. Chemokines activated by CREAP include IL-8 and Exodus1/MIP3 alpha and chemokines activated by CRE including MIP-1 beta (Proffitt et al., 1995, Gene 152:173-179; and Zhang et al., 2002; J. Biol Chem, 277:19042-19048).

“Subject” refers to any human or nonhuman organism.

In its broadest sense, the term “substantially similar” or “equivalent”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95%, still more preferably at least 99%.

A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄₁, 1 mM EDTA at 50° C. with washing in 0.1×SSC, 0.1% SDS at 65° C., yet still encodes a functionally equivalent gene product. Generally, hybridization conditions may be highly stringent or less highly stringent. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described in Krause and Aaronson (1991), Methods in Enzymology, 200:546-556 in addition to Maniatis et al., cited above.

“Elevated transcription of mRNA” refers to a greater amount of messenger RNA transcribed from the natural endogenous human gene encoding a CREAP polypeptide of the present invention in an appropriate tissue or cell of an individual suffering from a pathological condition related to abnormal activation of CRE-dependent gene expression or abnormal activation of chemokines compared to control levels, in particular at least about twice, preferably at least about five times, more preferably at least about ten times, most preferably at least about 100 times the amount of mRNA found in corresponding tissues in subjects who do not suffer from such a condition. Such elevated level of mRNA may eventually lead to increased levels of protein translated from such mRNA in an individual suffering from said condition as compared with a healthy individual.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and the like.

“Heterologous” as used herein means “of different natural origin” or represents a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g. a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.”

“Plasmids” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” refers to DNA sequences, such as initiator sequences, enhancer sequences, and promoter sequences, which induce, repress, or otherwise control the transcription of protein encoding nucleic acid sequences to which they are operably linked.

As used herein, “human transcriptional control sequences” are any of those transcriptional control sequences normally found associated with a human gene encoding any one of more of the CREAP proteins of the present invention as it is found in the respective human chromosome.

As used herein, “non-human transcriptional control sequence” is any transcriptional control sequence not found in the human genome.

As used herein, a “chemical derivative” of a polypeptide of the invention is a polypeptide of the invention that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th ed., Mack Publishing Co., Easton, Pa. (1980).

The instant invention is based on the surprising discovery that the protein previously referred to in public sequence databases as “KIAA0616” and heretofore of unknown function, is a CRE-activating protein. Referred to herein as CREAP1, in addition to activating CRE-dependent transcription in general, this polypeptide can also induce a variety of disease-associated genes such as chemokines, enzymes such as PEPCK and growth factors such as amphiregulin.

In addition, a search of public databases indicates that two cDNAs and proteins previously deposited (albeit with errors and/or only partial sequence) without any reference to function, XP_{—}117201 and FLJ00364, encode proteins with activities similar to CREAP1. As such, the present invention includes heretofore undisclosed accurate nucleotide sequences which encode polypeptides designated herein as CREAP 2 and CREAP3 and which belong to a new CREAP family of proteins, as will be outlined in detail herein.

Thus, the present invention provides isolated polypeptides comprising amino acid sequence as set forth in SEQ ID NO:16 and SEQ ID NO:25. Furthermore, the invention provides isolated polypeptides consisting of amino acid sequences set forth in SEQ ID NO:16 and SEQ ID NO:25. Such polypeptides may be, for example, a fusion protein including the amino acid sequence of CREAP 2 or CREAP 3. Fusion proteins comprising CREAP 1 are also contemplated herein.

The invention also includes isolated nucleic acid or nucleotide molecules, preferably DNA molecules, in particular encoding CREAP proteins, particularly, CREAP 2 or CREAP 3. Preferably, an isolated nucleic acid molecule, preferably a DNA molecule, of the present invention encodes a polypeptide comprising the amino acid sequence set forth in SEQ ID NO:16 or SEQ ID NO:25. Likewise preferred is an isolated nucleic acid molecule, preferably a DNA molecule, encoding a polypeptide consisting of the amino acid sequence set forth in SEQ ID NO:16 or SEQ ID NO:25.

The invention also encompasses: (a) vectors that comprise a nucleotide sequence of a CREAP protein, particularly human CREAP1, CREAP2 or CREAP3 or a fragment thereof and/or their complements (i.e., antisense); (b) vector molecules, preferably vector molecules comprising transcriptional control sequences, in particular expression vectors, which comprise coding sequences of any of the foregoing CREAP proteins operatively associated with a regulatory element that directs the expression of the coding sequences; and (c) genetically engineered host cells that contain a vector molecule as mentioned herein or at least a fragment of any of the foregoing nucleotide sequences operatively associated with a regulatory element that directs the expression of the coding sequences in the host cell. As used herein, regulatory elements include, but are not limited to, inducible and non-inducible promoters, enhancers, operators and other elements known to those skilled in the art that drive and regulate expression. Preferably, host cells can be vertebrate host cells, preferably mammalian host cells, such as human cells or rodent cells, such as CHO or BHK cells. Likewise preferred, host cells can be bacterial host cells, in particular E. coli cells.

Particularly preferred is a host cell, in particular of the above described type, which can be propagated in vitro and which is capable upon growth in culture of producing a CREAP polypeptide, in particular a polypeptide comprising or consisting of an amino acid sequence set forth in SEQ ID NOs:2, 16 or 25, wherein said cell comprises at least one transcriptional control sequence that is not a transcriptional control sequence of the natural endogenous human gene encoding said polypeptide, wherein said one or more transcriptional control sequences control transcription of a DNA encoding said polypeptides.

The invention also includes fragments of any of the nucleic acid sequences disclosed herein. Fragments of the nucleic acid sequences encoding a CREAP polypeptide may be used as a hybridization probe for a cDNA library to isolate the full length gene and to isolate other genes which have a high sequence similarity to a CREAP gene of similar biological activity. Probes of this type preferably have at least about 30 bases and may contain, for example, from about 30 to about 50 bases, about 50 to about 100 bases, about 100 to about 200 bases, or more than 200 bases. The probe may also be used to identify a CDNA clone corresponding to a full length transcript and a genomic clone or clones that contain a complete CREAP gene including regulatory and promoter regions, exons, and introns. An example of a screen comprises isolating the coding region of a CREAP gene by using the known DNA sequence to synthesize an oligonucleotide probe. Labeled oligonucleotides having a sequence complementary to that of the gene of the present invention are used to screen a library of human cDNA, genomic DNA or mRNA to determine to which members of the library the probe hybridizes.

In addition to the gene sequences described above, homologs of such sequences are disclosed herein, specifically, CREAP proteins from Drosophila, mouse and Fugu rubripres have been identified (see Examples, below); Additional homologs may be identified and readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

For example, the isolated nucleotide sequence of the present invention encoding a CREAP polypeptide may be labeled and used to screen a cDNA library constructed from mRNA obtained from the organism of interest. Hybridization conditions will be of a lower stringency when the cDNA library was derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, for example, Sambrook et al. cited above.

Further, a previously unknown differentially expressed gene-type sequence may be isolated by performing PCR using two degenerate oligonucleotide primer pools designed on the basis of amino acid sequences within the gene of interest. The template for the reaction may be cDNA obtained by reverse transcription of mRNA prepared from human or non-human cell lines or tissue known or suspected to express a differentially expressed gene allele.

The PCR product may be subcloned and sequenced to ensure that the amplified sequences represent the sequences of a differentially expressed gene-like nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to screen a genomic library.

PCR technology may also be utilized to isolate full length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

In cases where the gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to disease symptoms related to inflammation or immune response. Mutant alleles and mutant allele products may then be utilized in the diagnostic assay systems described below.

A cDNA of the mutant gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probed to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described, below. (For screening techniques, see, for example, Harlow, E. and Lane, eds., 1988, “Antibodies: A Laboratory Manual”, Cold Spring Harbor Press, Cold Spring Harbor.) In cases where the mutation results in an expressed gene product with altered function (e.g., as a result of a missense mutation), a polyclonal set of antibodies are likely to cross-react with the mutant gene product. Library clones detected via their reaction with such labeled antibodies can be purified and subjected to sequence analysis as described above.

The present invention includes those proteins or fragments thereof encoded by nucleotide sequences set forth in any of SEQ ID NOs:1,15,24,26,28,31.

Furthermore, the present invention includes proteins that represent functionally equivalent gene products. Such an equivalent differentially expressed gene product may contain deletions, additions or substitutions of amino acid residues within the amino acid sequence encoded by the differentially expressed gene sequences described, above, but which result in a silent change, thus producing a functionally equivalent differentially expressed gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent,” as utilized herein, may refer to a protein or polypeptide capable of exhibiting a substantially similar in vivo or in vitro activity as the endogenous differentially expressed gene products encoded by the differentially expressed gene sequences described above. “Functionally equivalent” may also refer to proteins or polypeptides capable of interacting with other cellular or extracellular molecules in a manner substantially similar to the way in which the corresponding portion of the endogenous differentially expressed gene product would. For example, a “functionally equivalent” peptide would be able, in an immunoassay, to diminish the binding of an antibody to the corresponding peptide (i.e., the peptide the amino acid sequence of which was modified to achieve the “functionally equivalent” peptide) of the endogenous protein, or to the endogenous protein itself, where the antibody was raised against the corresponding peptide of the endogenous protein. An equimolar concentration of the functionally equivalent peptide will diminish the aforesaid binding of the corresponding peptide by at least about 5%, preferably between about 5% and 10%, more preferably between about 10% and 25%, even more preferably between about 25% and 50%, and most preferably between about 40% and 50%.

Data disclosed herein indicate particular polypeptide fragments are critical to the activity of the CREAP family of proteins. For CREAP1-3, these regions are particularly the conserved amino terminal 200 amino acids and the carboxy terminal 100 amino acids each region of which as several conserved domains. Particularly preferred polypeptides of the present invention are those which comprise amino acid sequences corresponding to or contained within the evolutionally conserved regions such as, e.g., the terminal 75 amino acids of each protein; e.g., the region from a.a. 1 to 75, more specifically, the amino acid fragment 1-68 for CREAP1, the amino acid fragment 1-74 for CREAP2 and the amino acid fragment 1-66 for CREAP3.

Thus, these CREAP peptide fragments as well as fragments of the nucleic acids encoding the active portion of the CREAP polypeptides disclosed herein, and vectors comprising said fragments, are also within the scope of the present invention. As used herein, a fragment of the of the nucleic acid encoding the active portion of the CREAP polypeptides refers to a nucleotide sequence having fewer nucleotides than the nucleotide sequence encoding the entire amino acid sequence of a CREAP polypeptide and which encodes a peptide having an activity of a CREAP protein (i.e., a peptide having at least one biological activity of a CREAP protein) as defined herein. Generally, the nucleic acid encoding a peptide having an activity of a CREAP protein will be selected from the bases encoding the mature protein. However, in some instances, it may be desirable to select all or part of a peptide from the leader sequence portion of the nucleic acids of a CREAP protein. These nucleic acids may also contain linker sequences, modified restriction endonuclease sites and other sequences useful for molecular cloning, expression or purification or recombinant peptides having at least one biological activity of a CREAP protein. CREAP peptide fragments as well as nucleic acids encoding a peptide fragment having an activity of a CREAP protein may be obtained according to conventional methods.

In addition, antibodies directed to these peptide fragments may be made as described hereinabove. Modifications to these polypeptide fragments (e.g., amino acid substitutions) which may increase the immunogenicity of the peptide, may also be employed. Similarly, using methods familiar to one of skill in the art, said peptides of the CREAP proteins may be modified to contain signal or leader sequences or conjugated to a linker or other sequence to facilitate molecular manipulations.

The polypeptides of the present invention may be produced by recombinant DNA technology using techniques well known in the art. Therefore, there is provided a method of producing a polypeptide of the present invention, which method comprises culturing a host cell having incorporated therein an expression vector containing an exogenously-derived polynucleotide encoding a polypeptide comprising an amino acid sequence as set forth in SEQ ID NOs:2,16,25,27,29,and 30, preferably SEQ ID NOs 2, 16 and 25, under conditions sufficient for expression of the polypeptide in the host cell, thereby causing the production of the expressed polypeptide. Optionally, said method further comprises recovering the polypeptide produced by said cell. In a preferred embodiment of such a method, said exogenously-derived polynucleotide encodes a polypeptide consisting of an amino acid sequence set forth in SEQ ID NO: 2,16,25,27,29,and 30. Preferably, said exogenously-derived polynucleotide comprises the nucleotide sequence as set forth in any of SEQ ID NOs: 1,15,24,26,28 and 31.

Thus, methods for preparing the polypeptides and peptides of the invention by expressing nucleic acid encoding respective polypeptide sequences are described herein. Methods that are well known to those skilled in the art can be used to construct expression vectors containing protein-coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, RNA capable of encoding differentially expressed gene protein sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

A variety of host-expression vector systems may be utilized to express the differentially expressed gene coding sequences of the invention. Such host-expression systems represent vehicles by which the coding sequences of interest may be produced and subsequently purified, but also represent cells which may, when transformed or transfected with the appropriate nucleotide coding sequences, exhibit the differentially expressed gene protein of the invention in situ. These include but are not limited to microorganisms such as bacteria (e.g., E. coli, B. subtilis) transformed with recombinant bacteriophage DNA, plasmid DNA or cosmid DNA expression vectors containing differentially expressed gene protein coding sequences; yeast (e.g. Saccharomyces, Pichia) transformed with recombinant yeast expression vectors containing the differentially expressed gene protein coding sequences; insect cell systems infected or transfected with recombinant virus expression vectors (e.g., baculovirus) containing the differentially expressed gene protein coding sequences; plant cell systems infected with recombinant virus expression vectors (e.g., cauliflower mosaic virus, CaMV; tobacco mosaic virus, TMV) or transformed with recombinant vectors, including plasmids, (e.g., Ti plasmid) containing protein coding sequences; or mammalian cell systems (e.g. COS, CHO, BHK, 293, 3T3) harboring recombinant expression constructs containing promoters derived from the genome of mammalian cells (e.g., metallothioneine promoter) or from mammalian viruses (e.g., the adenovirus late promoter; the vaccinia virus 7.5K promoter, or the CMV promoter).

Expression of the CREAP proteins of the present invention by a cell from a CREAP-encoding gene that is native to the cell can also be performed. Methods for such expression are detailed in, e.g., U.S. Pat. Nos. 5,641,670; 5,733,761; 5,968,502; and 5,994,127, all of which are expressly incorporated by reference herein in their entirety. Cells that have been induced to express CREAP by the methods of any of U.S. Pat. Nos. 5,641,670; 5,733,761; 5,968,502; and 5,994,127 can be implanted into a desired tissue in a living animal in order to increase the local concentration of CREAP in the tissue. Such methods have therapeutic implications for, e.g., neurodegenerative conditions in which loss of CREB function occurs and as such agonists and/or exogenous CREAP protein may be useful to prevent, treat or ameliorate said conditions.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable. In this respect, fusion proteins comprising hexahistidine tags may be used (Sisk et alk, 1994: J. Virol 68: 766-775) as provided by a number of vendors (e.g. Qlagen, Valencia, Calif.). Such vectors include, but are not limited, to the E. coli expression vector pUR278 (Ruther et al., 1983, EMBO J. 2:1791), in which the protein-encoding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

Promoter regions can be selected from any desired gene using vectors that contain a reporter transcription unit lacking a promoter region, such as a chloramphenicol acetyl transferase (“CAT”), or the luciferase transcription unit, downstream of restriction site or sites for introducing a candidate promoter fragment; i.e., a fragment that may contain a promoter. For example, introduction into the vector of a promoter-containing fragment at the restriction site upstream of the CAT gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well-known and readily available promoters, but also promoters that readily may be obtained by the foregoing technique, using a reporter gene.

Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus C“RSV”), and metallothionein promoters, such as the mouse metallothionein-I promoter.

In an insect system, Autographa californica nuclear polyhedrosis virus (AcNPV) is one of several insect systems that can be used as a vector to express foreign genes. The virus grows in Spodoptera frugiperda cells. The coding sequence may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the desired protein in infected hosts. (E.g., See Logan & Shenk, 1984, Proc. Natl. Acad. Sci. USA 81:3655-3659). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544). Other common systems are based on SV40, retrovirus or adeno-associated virus. Selection of appropriate vectors and promoters for expression in a host cell is a well-known procedure and the requisite techniques for expression vector construction, introduction of the vector into the host and expression in the host per se are routine skills in the art. Generally, recombinant expression vectors will include origins of replication, a promoter derived from a highly expressed gene to direct transcription of a downstream structural sequence, and a selectable marker to permit isolation of vector containing cells after exposure to the vector.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

The present invention also includes recombinant CREAP peptides and peptide fragments having an activity of a CREAP protein. The term “recombinant protein” refers to a protein of the present invention which is produced by recombinant techniques, wherein generally DNA encoding a CREAP active fragment is inserted into a suitable expression vector which is in turn used to transform a host cell to produce the heterologous protein, In particular, recombinant peptide fragments having an activity of a CREAP protein includes CREAP protein fragments comprising the conserved amino terminal 200 amino acids or the carboxy terminal 100 amino acids of CREAP1, 2 or 3. Said fragments include amino acid fragments 1-267 and 575-650 for CREAP1, amino acid fragments 1-280 and 615-693 for CREAP2 and amino acid fragments 1-279 and 545-619 for CREAP3 as well as fragments comprising regions from amino acids 1-75 in human CREAP1-3 as discussed above.

Recombinant proteins of the present invention also may include chimeric or fusion proteins of CREAP and different polypeptides which may be made according to techniques familiar to one of skill in the art (see, for example, Current Protocols in Molecular Biology; Eds Ausubel et al. John Wiley & Sons; 1992; PNAS 85:4879 (1988)).

For long-term, high-yield production of recombinant proteins, stable expression is preferred. For example, cell lines which stably express the differentially expressed gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci Which in turn can be cloned and expanded into cell lines. This method may advantageously be used to engineer cell lines that express the differentially expressed gene protein. Such engineered cell lines may be particularly useful in screening and evaluation of compounds that affect the endogenous activity of the expressed protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

An alternative fusion protein system allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, a protein of the present invention may be labeled, either directly or indirectly, to facilitate detection of a complex formed between the protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels.

Where recombinant DNA technology is used to produce a protein of the present invention for such assay systems, it may be advantageous to engineer fusion proteins that can facilitate labeling, immobilization, detection and/or isolation.

Indirect labeling involves the use of a protein, such as a labeled antibody, which specifically binds to a polypeptide of the present invention. Such antibodies include but are not limited to polyclonal, monoclonal, chimeric, single chain, Fab fragments and fragments produced by a Fab expression library.

It is also contemplated herein that the CREAP proteins disclosed herein are useful drug targets for the development of therapeutics for the treatment of pathological conditions related to abnormal activation of CRE-dependent gene expression or abnormal activation of chemokines. Such conditions include, but are not limited to, osteoarthritis, psoriasis, asthma, COPD, psoriasis, asthma, rheumatoid arthritis, cancer, pathological angiogenesis, diabetes, hypertension, chronic pain, and other inflammatory and autoimmune diseases as well as neurodegenerative conditions such as Alzheimer's Disease, Parkinson's Disease and Huntington Disease.

In addition to chemokines, data also indicate that CREAP proteins can induce other genes such as PEPCK and amphiregulin. Amphiregulin is an EGF like growth factor associated with cancer. PEPCK is the limiting factor in glucose synthesis and as such is required for gluconeogenesis, blockade of which is commonly thought to be a therapeutic approach to treating diabetes. As such, it is also contemplated herein that the pathological conditions that may be treated by the modulators of the present invention include conditions associated with abnormal activity or expression of these proteins.

In yet another aspect, the present invention relates to a method to identify modulators useful to treat, prevent or ameliorate the pathological conditions discussed above comprising: a) assaying for the ability of a candidate modulator to inhibit or enhance CREAP activity and/or inhibit or enhance CREAP expression in vitro, ex vivo or in vivo and which can further include b) assaying for the ability of an identified CREAP modulator to reverse the pathological effects observed in in vitro, ex vivo or in vivo models of said pathological conditions and/or in clinical studies with subjects with said pathological conditions.

Conventional screening assays (e.g., in vitro, ex vivo and in vivo) may be used to identify modulators that inhibit or enhance CREAP protein activity and/or inhibit or enhance CREAP expression. CREAP activity and CREAP levels can be assayed in a subject using a biological sample from the subject using conventional assay methods. CREAP gene expression (e.g. mRNA levels) may also be determined using methods familiar to one of skill in the art, including, for example, conventional Northern analysis or commercially available microarrays. Additionally, the effect of a test compound on CREAP levels and/or related regulatory protein levels can be detected with an ELISA antibody-based assay or fluorescent labelling reaction assay. These techniques are readily available for high throughput screening and are familiar to one skilled in the art.

Data gathered from these studies may be used to identify those modulators with therapeutic usefulness for the treatment of the pathological conditions discussed above; e.g. inhibitory substances could be further assayed in conventional in vitro or in vivo models of said pathological conditions and/or in clinical trials with humans with said pathological conditions according to conventional methods to assess the ability of said compounds to treat, prevent or ameliorate said pathological conditions in vivo.

The present invention, by making available critical information regarding the active portions of CREAP polypeptides, allows the development of modulators of CREAP function e.g., small molecule agonists or antagonists, by employing rationale drug design familiar to one of skill in the art.

In another aspect, the invention relates to a method to prevent, treat or ameliorate the pathological conditions described herein comprising administering to a subject in need thereof a pharmaceutical composition comprising an effective amount of a CREAP modulator. Such modulators include antibodies directed to the CREAP polypeptides or fragments thereof. In certain particularly preferred embodiments, the pharmaceutical composition comprises antibodies that are highly selective for human CREAP polypeptides or portions of human CREAP polypeptides. Antibodies to CREAP proteins may cause the aggregation of the protein in a subject and thus inhibit or reduce the activity of the protein. Such antibodies may also inhibit or decrease CREAP activity, for example, by interacting directly with active sites or by blocking access of substrates to active sites. CREAP antibodies may also be used to inhibit CREAP activity by preventing protein-protein interactions that may be involved in the regulation of CREAP proteins and necessary for protein activity. Antibodies with inhibitory activity such as described herein can be produced and identified according to standard assays familiar to one of skill in the art.

CREAP antibodies may also be used diagnostically. For example, one could use these antibodies according to conventional methods to quantitate levels of a CREAP protein in a subject; increased levels could, for example, indicate excessive activation of CRE-dependent gene expression (e.g. activation of genes that have CRE in their promoter regions ) and could possibly indicate the degree of excessive activation and corresponding severity of related pathological condition. Thus, different CREAP levels could be indicative of various clinical forms or severity of pathological conditions related to abnormal CRE-dependent gene expression or abnormal activation of chemokines. Such information would also be useful to identify subsets of patients suffering from a pathological condition that may or may not respond to treatment with CREAP modulators.

It is contemplated herein that monitoring CREAP levels or activity and/or detecting CREAP expression (mRNA levels) may be used as part of a clinical testing procedure, for example, to determine the efficacy of a given treatment regimen. For example, patients to whom drugs have been administered would be evaluated and the clinician would be able to identify those patients in whom CREAP levels, activity and/or expression levels are higher than desired (i.e. levels higher or lower than levels in control patients not experiencing a related disease state or in patients in whom a disease state has been sufficiently alleviated by clinical intervention). Based on these data, the clinician could then adjust the dosage, administration regimen or type of medicinal prescribed.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment of a given pathological condition.

As the CREAP gene family contains a critical region of high conservation, peptide mimetics of CREAP proteins would also be predicted to act as CREAP modulators. Thus, one embodiment of this invention are peptides derived or designed from CREAP family proteins which block CREAP function. These mimetics would be predicted to be able to block function of all the highly related CREAP proteins. Suitable peptide mimetics to CREAP proteins can be made according to conventional methods based on an understanding of the regions in the polypeptides required for CREAP protein activity. Briefly, a short amino acid sequence is identified in a protein by conventional structure function studies such as deletion or mutation analysis of the wild-type protein. Once critical regions are identified, it is anticipated that if they correspond to a highly conserved potion of the protein that this region will be responsible for a critical function (such as protein-protein interaction). A small synthetic mimetic that is designed to look like said critical region would be predicted to compete with the intact protein and thus interfere with its function. The synthetic amino acid sequence could be composed of amino acids matching this region in whole or in part. Such amino acids could be replaced with other chemical structures resembling the original amino acids but imparting pharmacologically better properties, such as higher inhibitory activity, stability, half-life or bioavailability.

Suitable antibodies to CREAP proteins or related regulatory proteins can be obtained from a commercial source or produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to the CREAP polypeptides discussed herein, various host animals may be immunized by injection with the polypeptides, or a portion thereof. Such host animals may include, but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Altematively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the CREAP polypeptides or related regulatory proteins, or fragments thereof.

The most commonly used reporter molecules are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

In another embodiment, nucleic acids comprising a sequence encoding a CREAP protein or functional derivative thereof are administered for therapeutic purposes, by way of gene therapy. Gene therapy refers to therapy performed by the administration of a nucleic acid to a subject. In this embodiment of the invention, the nucleic acid produces its encoded protein that mediates a therapeutic effect by promoting normal CRE-dependent gene expression or normal activation of chemokines.

Any of the methods for gene therapy available in the art can be used according to the present invention. Exemplary methods are described below.

In a preferred aspect, the therapeutic comprises a CREAP nucleic acid that is part of an expression vector that expresses a CREAP protein or fragment or chimeric protein thereof in a suitable host. In particular, such a nucleic acid has a promoter operably linked to the CREAP coding region, said promoter being inducible or constitutive, and, optionally, tissue-specific. In another particular embodiment, a nucleic acid molecule is used in which the CREAP coding sequences and any other desired sequences are flanked by regions that promote homologous recombination at a desired site in the genome, thus providing for intrachromosomal expression of a CREAP nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., 1989, Nature 342:435-438).

In a specific embodiment, a viral vector that contains a CREAP nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The CREAP nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a CREAP nucleic acid is introduced into the cells such that it is expressible by the cells or their progeny, and the recombinant cells are then administered in vivo for therapeutic effect. In a specific embodiment, stem or progenitor cells are used. Any stem-and/or progenitor cells that can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (see, e.g., WO 94/08598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique that provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

This invention also relates to the use of polynucleotides of the present invention as diagnostic reagents. In particular, the invention relates to a method for the diagnosis of a pathological condition associated with abnormal activation of CRE-dependent gene expression or abnormal activation of chemokines which comprises:

Detecting abnormal, e.g., elevated transcription of messenger RNA transcribed from a natural endogenous human gene encoding a polypeptide consisting of an amino acid sequence set forth in SEQ ID NOs:2,16,25 in an appropriate tissue or cell from a human, wherein said abnormal transcription is diagnostic of said human's suffering from a condition described above. In particular, said natural endogenous human gene comprises the nucleotide sequence set forth in SEQ ID NOs: 1,15,24. In a preferred embodiment such a method comprises contacting a sample of said appropriate tissue or cell or contacting an isolated RNA or DNA molecule derived from that tissue or cell with an isolated nucleotide sequence of at least about 20 nucleotides in length that hybridizes under high stringency conditions with the isolated nucleotide sequence encoding a polypeptide consisting of an amino acid sequence set forth in SEQ ID NOs:2,16,25. Detection of elevated transcription would indicate that the subject is a suitable candidates for treatment with one or more CREAP modulators.

Detection of a mutated form of a CREAP protein which is associated with a dysfunction will provide a diagnostic tool that can add to, or define, a diagnosis of a disease, or susceptibility to a disease, which results from under-expression, over-expression or altered spatial or temporal expression of a CREAP gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Hybridizing amplified DNA to labeled nucleotide sequences encoding a CREAP polypeptide of the present invention can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (e.g., Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401). In another embodiment, an array of oligonucleotides probes comprising nucleotide sequence encoding a CREAP polypeptide of the present invention or fragments of such a nucleotide sequence can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to disease through detection of mutation in a CREAP gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well known to those of skill in the art. Such assay methods include radioimmunoassays, competitive-binding assays, Western Blot analysis and ELISA assays.

Thus in another aspect, the present invention relates to a diagnostic kit which comprises:
(a) a polynucleotide of the present invention, preferably the nucleotide sequence of SEQ ID NOs:1,15 or 24, or a fragment thereof;
(b) a nucleotide sequence complementary to that of (a);
(c) a polypeptide of the present invention, preferably the polypeptide of SEQ ID NOs:2,16,25 or a fragment thereof;
(d) an antibody to a polypeptide of the present invention, preferably to the polypeptide of SEQ ID NOs:2,16,25; or
(e) a peptide mimetic to a CREAP protein, preferably of SEQ ID NO 2, 16 or 25.

It will be appreciated that in any such kit, (a), (b), (c), (d) or (e) may comprise a substantial component. Such a kit will be of use in diagnosing a disease or susceptibility to a disease, particularly to a disease or pathological condition associated with abnormal activation of CRE-dependent gene expression or abnormal activation of chemokines. It is also contemplated that said kit could comprise components (a)-(e) designed to detect levels of a CREAP related regulatory proteins or proteins modified by CREAP as discussed herein.

The nucleotide sequences of the present invention are also valuable for chromosome localization. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

The pharmaceutical compositions of the present invention may also comprise substances that inhibit the expression of CREAP proteins at the nucleic acid level. Such molecules include ribozymes, antisense oligonucleotides, triple helix DNA, RNA aptamers, siRNA, and double or single stranded RNA directed to an appropriate nucleotide sequence of a CREAP nucleic acid. These inhibitory molecules may be created using conventional techniques by one of skill in the art without undue burden or experimentation. For example, modifications (e.g. inhibition) of gene expression can be obtained by designing antisense molecules, DNA or RNA, to the control regions of a gene encoding a CREAP polypeptide discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the gene for CREAP1, CREAP2 or CREAP3.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double stranded RNA technologies. A description of such technology may be found in WO 99/32619 which is hereby incorporated by reference in its entirety. In addition, siRNA technology has also proven useful as a means to inhibit gene expression (Cullen, BR Nat. Immunol. 2002 July;3(7):597-9).

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

In addition to the above described methods for inhibiting CREAP expression, it is contemplated herein that one could identify and employ small molecules or other natural products to inhibit the transcription in vivo of the polypeptides discussed herein. For example, one of skill in the art could establish an assay for CREAP1, CREAP2 or CREAP3 that can be easily applied to samples from the culture media of a cell line using conventional methods. Using this assay, cell lines would be screened to find ones that express the CREAP protein of interest. These cell lines could be cultured in, for example, 96 well plates. A comparison of the effects of some known modifiers of gene expression e.g. dexamethasone, phorbol ester, heat shock on primary tissue cultures and the cell lines will allow the selection of the most appropriate cell line to use. The screen would then merely consist of culturing the cells for a set length of time with a different compound added to each well and then assaying for CREAP activity/mRNA level.

In order to faciliate the detection of CREAP in the assay described above, luciferase or other commercially available fluorescent protein could be genetically fused as an appropriate marker protein to the promoter of CREAP1, CREAP 2 or CREAP3. Sequences upstream of the ATG of, e.g. the promoter of CREAP1, can be identified from genomic sequence data by using the sequence from GenBank accession number NM_{—}025021 to BLAST against the NCBI genomic sequence. (Currently the GenBank Accession number for the genomic contigue sequence for CREAP1 is NT_{—}011295) This gives at least 5 kb upstream of the ATG of CREAP1 that does not contain any unknown bases. Two pairs of nested PCR primers to amplify a fragment of 2 kb or longer from human genomic DNA can be readily designed and tested. The promoter fragment can be readily inserted into any promoter-less reporter gene vector designed for expression in human cells (e.g. Clontech promoter-less enhanced fluorescent protein vector pECFP-1, pEGFP-1, or pEYFP, Clontech, Palo Alto, Calif.). The screen would then consist of culturing the cells for an appropriate length of time with a different compound added to each well and then assaying for reporter gene activity. Promising compounds would then be assayed for effects on CREAP1 activity and/or mRNA level in vivo using the in vivo models of the pathological conditions previously described. Additional method details such as appropriate culturing time, culture conditions, reporter assays and other methodologies that can be used to identify small molecules or other natural products useful to inhibit the transcription of CREAP proteins in vivo would be familiar to one of skill in the art.

In addition, the cDNA encoding CREAP proteins and/or the CREAP proteins themselves can be used to identify other proteins, e.g. kinases, proteases or transcription factors, that are modified or indirectly activated in a cascade by CREAP proteins. Proteins thus identified can be used, for example, for drug screening to treat the pathological conditions discussed herein. To identify these genes that are downstream of CREAP proteins, it is contemplated, for example, that one could use conventional methods to treat animals in disease state models with a specific CREAP inhibitor, sacrifice the animals, remove relevant tissues and isolate total RNA from these cells and employ standard microarray assay technologies to identify message levels that are altered relative to a control animal (animal to whom no drug has been administered).

In addition, conventional in vitro or in vivo assays may be used to identify possible genes that lead to over expression of CREAP proteins. These related regulatory proteins encoded by genes thus identified can be used to screen drugs that might be potent therapeutics for the treatment of the pathological conditions discussed herein. For example, a conventional reporter gene assay could be used in which the promoter region of a CREAP protein is placed upstream of a reporter gene, the construct transfected into a suitable cell (for example from ATCC, Manassas, Va.) and using conventional techniques, the cells assayed for an upstream gene that causes activation of the CREAP promoter by detection of the expression of the reporter gene.

It is contemplated herein that one can inhibit the function and/or expression of a gene for a related regulatory protein or protein modified by a CREAP protein as a way to treat the pathological conditions discussed herein by designing, for example, antibodies to these proteins or peptide mimetics and/or designing inhibitory antisense oligonucleotides, triple helix DNA, ribozymes, siRNA, double or single stranded RNA and RNA aptamers targeted to the genes for such proteins according to conventional methods. Pharmaceutical compositions comprising such inhibitory substances for the treatment of said pathological conditions are also contemplated.

An additional embodiment of the invention relates to the administration of a pharmaceutical composition, in conjunction with a pharmaceutically acceptable carrier, excipient or diluent, for treatment of any of the pathological conditions discussed herein. Such pharmaceutical compositions may comprise CREAP proteins, or fragments thereof, antibodies to CREAP polypeptides or peptide fragments, mimetics, and/or CREAP modulators (e.g. agonists, antagonists, or inhibitors of CREAP expression and/or function). The compositions may be administered alone or in combination with at least one other agent, such as stabilizing compound, which may be administered In any sterile, biocompatible pharmaceutical carrier, including, but not limited to, saline, buffered saline, dextrose, and water. The compositions may be administered to a patient alone, or in combination with other agents, drugs or hormones.

Pharmaceutical compositions comprising CREAP proteins or fragments thereof may be administered when deemed medically beneficial by one of skill in the art, e.g. in conditions wherein agonists of CREAP function have a therapeutic effect such as neurodegenerative disorders such as Alzheimer's , Parkinson's and Huntington diseases. Such pharmaceutical compositions for use in accordance with the present invention may be formulated in a conventional manner using one or more physiologically acceptable carriers or excipients.

The pharmaceutical compositions disclosed herein useful for preventing, treating or ameliorating pathological conditions related to abnormal CRE-dependent gene expression or abnormal activation of chemokines are to be administered to a patient at therapeutically effective doses. A therapeutically effective dose refers to that amount of the compound sufficient to result in the prevention, treatment or amelioration of said conditions.

Compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient useful to prevent, treat or ameliorate a particular pathological condition of interest. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The following examples further illustrate the present invention and are not intended to limit the invention.

The following materials and methods were performed to conduct Examples 1-5 below:

Assembly of a Collection of Human Full-length cDNA Clones

We have archived and sequenced, at the 5′ end, about 170,000 clones from multiple high-quality full-length cDNA libraries made from mRNAs of 33 human tissue types. Using a proprietary bioinformatics pipeline, we have identified all the CDNA clones that have the initial ATG codon for an ORF, either experimentally defined or conceptually predicted, and thus potentially represent the full-length transcripts. A total of 20,702 clones, within pCMVSport6 vector (Invitrogen, Carlsbad, Calif.), were rearrayed from the archived clone set using a Q-bot (Genetix Limited, Hampshire, United Kingdom), into 384-well Genetix plates containing 60 ul Luria broth (LB). Based on bioinformatics analysis of the 5′ sequences of these 20, 702 clones, they are derived from approximately 11,000 genes with strong support for their structure and existence, although most of them have no function, and 6,000 potential novel sequences are not yet in the public cDNA databases.

The arrayed clones are replicated to produce multiple copies for archiving. One copy is used to produce miniprep DNA using a QIAGEN BioRobot 8000 (Qiagen, Valencia, Calif.). The DNA samples are eluted into 96-well UV-plates (Coming, Acton, Mass.) and their concentration and yield is determined by measuring the OD260 value on a SPECTRAmax 190 (Molecular Devices, Sunnyvale, Calif.). The resulting 20, 702 DNA samples are then aliquoted to produce multiple copies for archiving (at 80 pg/well in TE buffer) and cell-based assays in 384-well plates (at 50 ng/well in OPTI-MEM cell culture medium (Invitrogen). Plates are sealed and stored at −20° C.

Genome-wide Screening for Activators of Cyclic AMP Response Element

Hela cells (ATCC, Manassas, Va.) grown in 225 ml tissue culture flasks are trypsinized and diluted to 10⁵ cells/ml in DMEM medium (Invitrogen). The cell suspension is then dispensed into 384-well tissue culture plates with a Multi-drop 384 (Thermo Labsystems, Beverly, Mass.) at 30 μl/well. After incubation overnight, a mixture composed of 0.25 ul Fugene 6 transfection reagent (Roche Applied Biosciences), 6 μl of OPTI-MEM medium containing 50 ng of pCRE-Luc plasmid construct (Stratagene) and 50 ng of individual cDNA plasmid from the clone collection is added to each well of 384-well plates using a Biomek FX liquid handling robot (Beckman Coulter). Forty hours post transfection, luciferase activity in each well is measured using the BrightGlo Luciferase Assay System (Promega, Madison, Wis.) on a LUMINOSKAN Ascent luminometer (Thermo Labsystems) according to manufacturer's protocols. Raw luciferase data are processed by an in-house data processing and analysis system specifically designed for managing high-throughput gene functionalization project. The whole assays are conducted in duplicate to produce 41, 404 data points, each corresponding to a miniaturized transfection experiment with an individual cDNA clone in a single well.

HTS Hits Confirmation and Validation

For each set of the duplicated 20,702 data points, Z score (calculated as fold of activation divided by the standard deviation of the population) and fold activation against the population median are calculated and deposited into an annotated searchable database. Potential activators are selected based upon two criteria: (1) Z scores larger than 3.0 in either assays and (2) fold increase in luciferase/median is greater than 8.0 in both assay. A total of 85 clones (0.4% of total clones) were identified based upon the above criteria. The DNA samples for these hits are retrieved from the clone archive and re-transformed into bacterial strain XL-10 Gold (Stratagene). Individual colonies for each sample are picked and DNA mini-preps are performed. A portion of mini-prep DNA samples is sequenced from the 5′ end for clone verification. The remaining samples are used for hit validation in which they are manually transfected together with the pCRE-Luc reporter construct and pRL-SV40 plasmid (Promega) encoding Renilla luciferase under control of the SV40 early promoter into Hela cells followed by a Dual-luciferase assay (Promega) according to the manufacturer's suggestions.

Northern Blot Analysis and In Vitro Transcription and Translation Analysis

The pCMVSport6 plasmid containing CREAP1 cDNA is digested by EcoRI and NotI, the insert is gel purified using a Qiagen DNA gel extraction kit and labeled with Enzo random prime DNA labeling systems by following the vendor's manual (Bio-11-dCTP deoxynucleotide pack, Cat.# 42723, Enzo Biochem, Farmingdale, N.Y.). Briefly, 200 ng CREAP1 fragment, or 100 ng of β actin cDNA (Clontech) is denatured at 100° C. for 10 minutes, cooled on ice for 3-5 minutes, and then mixed with 5 ul 10× hexamer random primer, 5 μl dCTP-11-Bio mix and 1 μl Klenow fragment and incubated at 37° C. for 4 hrs. The probes are hybridized to a Multiple Tissue mRNA Northern blot membrane (Clontech) according to suggested protocols. Signal detection is achieved by utilizing a biotin detection kit (Ambion, Austin, Tex.). The membrane is exposed to X-ray film from 10 to 30 seconds. After initial exposure, the membrane is stripped and re-probed with a beta actin probe (Clontech) to normalize the expression level.

In vitro transcription and translation of CREAP1 protein is conducted with TNT SP6 Quick Coupled Transcription and Translation System (Promega) following the vendor's manual. The translation products are separated in a Nupage precast gel (4-20%) (Invitrogen), transferred to a nitrocellulose membrane and detected by the Transcend non-radioactive detection system (Promega) according to manufacturer's instructions.

CREAP1-CREB Signaling Pathway Analysis

For in vivo kinase assay, activation domains of CREB or ATF2 transcription factors fused with the yeast GAL4 DNA binding domain (1-147 Amino Acids) constructs are used (Stratagene, PathDetect In Vivo Signal Transduction Pathway trans-reporting Systems). The HLR cell line that contains a 5× GAL4 DNA binding element and TATA box driving luciferase reporter is used per manufacturer's protocol (Stratagene). 10⁴ HLR cells are split into each well of 96 well tissue culture plates. After 16 hours, cells are transfected with 100 ng of Creb-GAL4 or ATF2-GAL4 fusion constructs, 30 ng of Renilla luciferase control plasmid together with 100 ng of pCMVSPORT6, pCMVSPORT-CREAP1, pFC-PKA or pFC-MEKK (Stratagene) activator plasmids respectively. Transfection is done with Fugene6 reagent (Roche Molecular Biochemicals, Basel, Switzerland) according to the manufacturers manual. Forty hours after transfection, a Dual-Glo Luciferase assay (Promega) is conducted using the manufacturer's protocol.

For dominant negative CREB assay, CREB dominant negative constructs (Non-phophorylatable S133A mutant or DNA binding domain K287L mutant K-Creb) are used (Clontech, Cat.# K6014-1). Above transfection and luciferase assay procedure are followed with some modifications according to the manufacturer. Hela cells, pCMVSPORT6, pCMV-CREAP1, pS133A-Creb or pK-Creb constructs are utilized for transfection.

Functional Analysis of CREAP1 Protein Deletions

CREAP1 protein amino acids 1-170, 1-356, 1494, 1-580 and 170-650 are inserted into pFlag-CMV4 expression vector (Sigma, St. Louis, Mo.) by utilizing PCR strategy familiar to one of skill in the art. 10⁴ Hela cells are split into each well of 96 well tissue culture plates. Cells are transfected 16 hours later with 100 ng of pCRE-Luc reporter construct, 30 ng of Renilla luciferase control plasmid together with 100 ng of pCMVSPORT6, pCMVSPORT-CREAP1 and different Flag-CREAP1 deletion fusion constructs respectively. Transfection is done with Fugene6 reagent (Roche Applied Biosciences) following the manufacturer's instructions. A Dual-Glo Luciferase assay (Promega) is conducted 40 hrs after transfection. Firefly luciferase counts are normalized to Renilla luciferase and plotted.

### Example 1

### Genome-wide Screening for Cyclic AMP Response Element Activator Genes

To identify cDNAs encoding proteins that could lead to CRE activation, we screened an annotated and indexed collection of 20,702 human cDNA clones, which are predicted to represent full-length transcripts for 11,000-16,000 individual genes in a miniaturized CRE-luciferase reporter system. The experiments were conducted in duplicate to produce a total of 41,404 data points, each corresponding to the luciferase activity from a transient protein over-expression assay, where about 3,000 Hela cells were transiently transfected with the cDNA clone of interest and a plasmid containing the firefly luciferase gene. Statistical analysis of the two data sets has generated a list of 85 clones that lead to at least 8 fold increase in lucifease activity compared to the population median in two of the duplicated primary screening experiments. In subsequent secondary verification experiments, when individual colonies for these clones were retrieved and subjected to similar assays but with Rennila luciferase under the control of SV40 promoter for data normalization, 14 clones were confirmed (data not shown). Hits obtained included a protein of heretofore unknown function, named KIAA0616 (Accession number NM_{—}025021) by the Kazusa DNA Research Institute. Based on our functional analysis of this protein, we renamed this protein CRE activating protein 1 or “CREAP1”, based on its ability to activate CRE in the transient overexpression luciferase reporter assay system described herein.

To further define the pathway or promoter specificity for CREAP1, it was tested against a group of various promoter-luciferase constructs in a similar assay system in Hela cells. These constructs could test the ability of CREAP1 to activate CREB, NFAT and NFkB transcription factor binding elements as well as authentic promoters for IL-8, VCAM, IL-24 and NPY. In addition, 3 luciferase vectors were included for background test and as a specificity control. Results indicate that CREAP1 is a CRE specific activator (data not shown).

### Example 2

### DNA Sequence and Amino Acid Sequence for CREAP1 Gene

The 2.4 kb cDNA insert in the active CREAP1 clone was sequenced from both strands according to conventional methods. Results indicate that the coding region of this gene is 1950 nucleotides and the amino acid sequence is predicted to be 650 amino acids. Bioinformatics analysis shows that CREAP1 contains no conserved protein functional domain (e.g. kinase ATP binding domain or transcription factor DNA binding domain) other than a proline rich domain from amino acid 379 to 448 in the middle of the molecule. The DNA sequence and amino acid sequence are shown below.

Full length confirmed DNA sequence of CREAP1:

| | |
|---|---|
| CCCCATTGACGCAAATGGGCGGTAGGCGTGTACGGTGGGAGGTCTATATAAGCAGAGCTCGTTTAGTG | (SEQ ID NO 1) |
| AACCGTCAGATCGCCTGGAGACGCCATCCACGCTGTTTTGACCTCCATAGAAGACACCGGGACCGATC | |
| CAGCCTCCGGACTCTAGCCTAGGCCGCGGGACGGATAACAATTTCACACAGGAAACAGCTATGACCAT | |
| TAGGCCTATTTAGGTGACACTATAGAACAAGTTTGTACAAAAAAGCAGGCTGGTACCGGTCCGGAATT | |
| CCCGGGAGGAGGAGGAGGTGGCGGCGAGAAGATGGCGACTTCGAACAATCCGCGGAAATTCAGCGAGA | |
| AGATCGCGCTGCACAATCAGAAGCAGGCGGAGGAGACGGCGGCCTTCGAGGAGGTCATGAAGGACCTG | |
| AGCCTGACGCGGGCCGCGCGGCTCCAGCTCCAGAAATCCCAGTACCTGCAACTGGGCCCCAGCCGAGG | |
| CCAGTACTATGGCGGGTCCCTGCCCAACGTGAACCAGATCGGGAGTGGCACCATGGACCTGCCCTTCC | |
| AGCCCAGCGGATTTCTGGGGGAGGCCCTGGCAGCGGCTCCTGTCTCTCTGACCCCCTTCCAATCCTCG | |
| GGCCTGGACACCAGCCGGACCACCCGGCACCATGGGCTGGTGGACAGGGTGTACCGGGAGCGTGGCCG | |
| GCTCGGCTCCCCACACCGCCGGCCCCTGTCAGTGGACAAACACGGACGGCAGGCCGACAGCTGCCCCT | |
| ATGGCACCATGTACCTCTCACCACCCGCGGACACCAGCTGGAGAAGGACCAATTCTGACTCCGCCCTG | |
| CACCAGAGCACAATGACGCCCACGCAGCCAGAATCCTTTAGCAGTGGGTCCCAGGACGTGCACCAGAA | |
| AAGAGTCTTACTGTTAACAGTCCCAGGAATGGAAGAGACCACATCAGAGGCAGACAAAAACCTTTCCA | |
| AGCAAGCATGGGACACCAAGAAGACGGGGTCCAGGCCCAAGTCCTGTGAGGTCCCCGGAATCAACATC | |
| TTCCCGTCTGCCGACCAGGAAAACACTACAGCCCTGATCCCCGCCACCCACAACACAGGGGGGTCCCT | |
| GCCCGACCTGACCAACATCCACTTCCCCTCCCCGCTCCCGACCCCGCTGGACCCCGAGGAGCCCACCT | |
| TCCCTGCACTGAGCAGCTCCAGCAGCACCGGCAACCTCGCGGCCAACCTGACGCACCTGGGCATCGGT | |
| GGCGCCGGCCAGGGAATGAGCACACCTGGCTCCTCTCCACAGCACCGCCCAGCTGGCGTCAGCCCCCT | |
| GTCCCTGAGCACAGAGGCAAGGCGTCAGCAGGCATCGCCCACCCTGTCCCCGCTGTCACCCATCACTC | |
| AGGCTGTAGCCATGGACGCCCTGTCTCTGGAGCAGCAGCTGCCCTACGCCTTCTTCACCCAGGCGGGC | |
| TCCCAGCAGCCACCGCCGCAGCCCCAGCCCCCGCCGCCTCCTCCACCCGCGTCCCAGCAGCCACCACC | |
| CCCGCCACCCCCACAGGCGCCCGTCCGCCTGCCCCCTGGTGGCCCCCTGTTGCCCAGCGCCAGCCTGA | |
| CTCGTGGGCCACAGCCGCCCCCGCTTGCAGTCACGGTACCGTCCTCTCTCCCCCAGTCCCCCCCAGAG | |
| AACCCTGGCCAGCCATCGATGGGGATCGACATCGCCTCGGCGCCGGCTCTGCAGCAGTACCGCACTAG | |
| CGCCGGCTCCCCGGCCAACCAGTCTCCCACCTCGCCAGTCTCCAATCAAGGCTTCTCCCCAGGGAGCT | |
| CCCCGCAACACACTTCCACCCTGGGCAGCGTGTTTGGGGACGCGTACTATGAGCAGCAGATGGCGGCC | |
| AGGCAGGCCAATGCTCTGTCCCACCAGCTGGAGCAGTTCAACATGATGGAGAACGCCATCAGCTCCAG | |
| CAGCCTGTACAGCCCGGGCTCCACACTCAACTACTCGCAGGCGGCCATGATGGGCCTCACGGGCAGCC | |
| ACGGGAGCCTGCCGGACTCGCAGCAACTGGGATACGCCAGCCACAGTGGCATCCCCAACATCATCCTC | |
| ACAGTGACAGGAGAGTCCCCCCCCAGCCTCTCTAAAGAACTGACCAGCTCTCTGGCCGGGGTCGGCGA | |
| CGTCAGCTTCGACTCCGACAGCCAGTTTCCCCTGGACGAACTCAAGATCGACCCCCTGACCCTCGACG | |
| GACTGCACATGCTCAACGACCCCGACATGGTTCTGGCCGACCCAGCCACCGAGGACACCTTCCGGATG | |
| GACCGCCTGTGAGCGGGCACGCCGGCACCCTGCCGCTCAGCCGTCCCGACGGCGCCTCCCCAGCCCGG | |
| GGACGGCCGTGCTCCGTCCCTCGCCAACGGCCGAGCTTGTGATTCTGAGCTTGCAATGCCGCCAAGCG | |
| CCCCCCGCCAGCCCGCCCCCGGTTGTCCACCTCCCGCGAAGCCCAATCGCGAGGCCGCGAGCCGGGCC | |
| GTCCACCCACCCGCCCGCCCAGGGCTGGGCTGGGATCGGAGGCCGTGAGCCTCCCGCCCCTGCAGACC | |
| CTCCCTGCAGTGGCTCCCTCGCCCCCAGCCCCGGGGCCTGAGCCGTCCCCTGTAAGATGCGGGAAGTG | |
| TCAGCTCCCGGCGTGGCGGGCAGGCTCAGGGGAGGGGCGCGCATGGTCCGCCAGGGCTGTGGGCCGTG | |
| GCGCATTTTCCGACTGTTTGTCCAGCTCTCACTGCCTTCCTTGGTTCCCGGTCCCCCAGCCCATCCGC | |
| CATCCCCAGCCCGTGGTCAGGTAGAGAGTGAGCCCCACGCCGCCCCAGGGAGGAGGCGCCAGAGCGCG | |
| GGGCAGACGCAAAGTGAAATAAACACTATTTTGACGGCAAAAAAAAAAAAAAAGGGCGGCCGCTCTAG | |
| AGTATCCCTCGAGGGGCCCAAG | |

Predicted Amino Acid Sequence of CREAP1 (650 amino acids):

| | |
|---|---|
| MATSNNPRKFSEKIALHNQKQAEETAAFEEVMKDLSLTRAARLQLQKSQYLQLGPSRGQYYGGSLPNV | (SEQ ID NO 2) |
| NQIGSGTMDLPFQPSGFLGEALAAAPVSLTPFQSSGLDTSRTTRHHGLVDRVYRERGRLGSPHRRPLS | |
| VDKHGRQADSCPYGTMYLSPPADTSWRRTNSDSALHQSTMTPTQPESFSSGSQDVHQKRVLLLTVPGM | |
| EETTSEADKNLSKQAWDTKKTGSRPKSCEVPGINIFPSADQENTTALIPATHNTGGSLPDLTNIHFPS | |
| PLPTPLDPEEPTFPALSSSSSTGNLAANLTHLGIGGAGQGMSTPGSSPQHRPAGVSPLSLSTEARRQQ | |
| ASPTLSPLSPITQAVAMDALSLEQQLPYAFFTQAGSQQPPPQPQPPPPPPPASQQPPPPPPPQAPVRL | |
| PPGGPLLPSASLTRGPQPPPLAVTVPSSLPQSPPENPGQPSMGIDIASAPALQQYRTSAGSPANQSPT | |
| SPVSNQGFSPGSSPQHTSTLGSVFGDAYYEQQMAARQANALSHQLEQFNMMENAISSSSLYSPGSTLN | |
| YSQAAMMGLTGSHGSLPDSQQLGYASHSGIPNIILTVTGESPPSLSKELTSSLAGVGDVSFDSDSQFP | |
| LDELKIDPLTLDGLHMLNDPDMVLADPATEDTFRMDRL | |

### Example 3

### Northern Blot and In Vitro Translation of CREAP1 Protein

To investigate CREAP1 gene expression in different human tissues, we conducted a Northern blot analysis using a randomly labeled CREAP1 probe. According to Northern blot analysis, two mRNAs were observed, 2.4 Kb and 7 Kb. The 2.4 Kb band is consistent with coding region size. The 7.0 Kb band may reflect an alternative splicing form of mRNA. Although expressed in most of the human tissues, CREAP1 mRNA is abundant in brain, heart, skeletal muscle and kidney (data not shown).

To test the accuracy of the predicted amino acid sequence of CREAP1, we used pCMVSPORT-CREAP1 as the template and conducted an in vitro transcription and translation reaction. After the in vitro translation products were resolved in SDS-PAGE, a single CREAP1 protein band was observed around 80 Kd, consistent with the idea that it contains 650 amino acids (data not shown).

### Example 4

### CREAP1 Acts through CREB

As CREAP1 strongly activates CRE promoter transcription, we next investigated whether CREAP1 works through the CREB pathway. To address this issue, an in vivo kinase assay was carried out using fusion constructs made up of transactivation domains of CREB or ATF2 transcription factors and the GAL4 DNA binding domain (amino acids 1-147) and the HLR cell line stably integrated with the PathDetect Trans-Reporter Plasmid (Stratagene). In this system, only those upstream regulators (presumably kinases) that activate the transactivation domains of CREB or ATF2 could drive luciferase reporter expression. Results indicate that CREAP1 strongly stimulates the transactivation of the CREB-GAL4 fusion molecule on the GAL4 promoter and its activity is even stronger than that of the PKA catalytic subunit, a canonical kinase that phosphorylates CREB. Interestingly, CREAP1 is unable to activate the ATF2-GAL4 fusion molecule whereas MEKK (an upstream kinase for ATF2 pathway, Stratagene kit manual) could stimulate ATF2 fusion more than 100 fold. This result demonstrates that CREAP1 is CREB pathway-specific upstream activator.

To further confirm this observation, two CREB dominant negative constructs (non-phosphorylatable S133A mutant or DNA binding domain mutant K287L, (Clontech) were utilized for a cotransfection assay. The experimental data showed that either the CREB S133A mutant or the K287L mutant could completely abolish the activation of CREAP1 on the CRE promoter, suggesting that CREAP1 specifically works upstream of the CREB signal transduction pathway and that both phosphorylation and DNA binding activity of CREB are required for CREAP1 signaling.

### Example 5

### Functional Analysis of CREAP1 Protein Intramolecular Domains

To dissect the functional domains within the CREAP1 molecule, CREAP1 protein fragments of amino acids 1-170, 1-356, 1-494, 1-580 and 170-650 were subcloned into pFlag-CMV4 vector by utilizing PCR based strategy and tested for function in a Dual Glo Luciferase assay as described above. Results indicate that the amino terminal fragment containing amino acids 1-170 of CREAP1 is important for its function, as K5 (aa 170-650) which lacks this amino terminus, lacked almost all stimulating activity. However, the 1-170 fragment alone (K1) is not sufficient for its function. On the other hand, the CREAP1 C terminus is dispensable for its function, as the K4 deletion (missing amino acids 581-650) retains almost all wild type activity. A comparison of the activity of K2 and K4 suggests that amino acids 356-580 (which has a proline rich domain) are very important for CREAP1 function, because removal of this portion from K4 (which results in K2) reduced the functional activity of CREAP1 by 10 fold (see Table 1 below).

TABLE 1
Function of CREAP fragments.

| | Amino acids of | | SEQ ID |
|---|---|---|---|
| Fragment | CREAP1 | Activity | NO # |
| K1 | 1-170, | Inactive | 32 |
| K2 | 1-356, | Inactive | 33 |
| K3 | 1-494, | Partially Active | 34 |
| K4 | 1-580 | Fully active | 35 |
| KS | 170-650 | Inactive | 36 |

The following materials and methods are used to perform the experiments listed below in Examples 6-9:

DNA Constructs

pGL-2-IL-8ₚ-Luc constructed in house using conventional methods (Roebuck, J. Interferon and Cytokine Res. 19:429-438 (1999)) contains a firefly luciferase gene driven by a 1.5 kb sequence containing the IL-8 promoter. pGL3B-IL-8ₚ-Luc is constructed by ligating the 1.5 kb human IL-8 promoter DNA excised by Hind III/Xhol digestion of pGL-2-IL-8ₚ-Luc and insertion into Hind III/Xho I digested pGL3Basic (Promega).

The pIL-8Luc reporter was constructed by insertion of the −1491 to +43 region of the human IL-8 gene into pGL3Basic vector (Promega, Inc). PCR was used to generate a wild type minimal IL-8 promoter as well as point mutants. Mutations in AP-1, C/EBP, NF-κB were as described by Wu et al. (Wu et al. J. Biol. Chem., 272:2396-2403 (1997)). The sequence of a putative CRE-like site TGACATAA was mutated to TCGATCAA. Promoter constructs carrying 6 concatamerised copies of CRE-like response element (pCREL-Luc) or 5 copies of CRE-like element TGACACAA found in human PEPCK and CAPL promoters (pCREL2-Luc) were prepared by ligating PCR amplified sequences into pTAL-Luc (BD Biosciences). All techniques were performed using conventional methods.

Construction of IL-8 Promoter Deletion and Point Mutated Variants

Polymerase chain reaction (PCR) is used to generate IL-8 promoter variants. PCR amplification cycles consist of: 2 min at 94° C., 5×[15 s at 94° C., 30 s at 55° C. and 15 s at 72° C.] and 20×[15 s at 94° C., 30 s at 65° C. and 15 s at 72° C.]. Advantage 2 DNA polymerase (BD Biosciences) is used for all the amplification steps. All the variants are amplified with a common antisense primer P2.1 with the nucleotide sequence (5′-GCCCAAGCTTTGTGCTCTGCTGTCTCTGAAAG-3′) (SEQ ID NO 3), corresponding to sequence +13-+43 of human IL-8 gene (Roebuck, J. Interferon and Cytokine Res. 19:429-438 (1999). A BamHI restriction site (underlined) is included in the sequence of all sense primers. PCR products are gel purified and ligated using Zero Blunt TOPO PCR cloning kit (Invitrogen). Sequence confirmed clones are excised by Hind III/BamH I from pCR-Blunt II-TOPO and ligated into Hind III/BamH I digested pGL3Basic. pIL-8p[deltaAP-1]-Luc, carrying truncated minimal IL-8 promoter lacking the AP-1 site is created by amplification with P2.1 and S3 (5′-GCCCTGAGGGGATGGGCCATCAG-3′) (SEQ ID NO 4), primers to generate a 157 nt product corresponding to sequence −114-+43 of human IL-8 gene.

Minimal IL-8 promoters carrying either wild type or mutated AP-1 sites are amplified with wtAP-1 (5′-CGCGGATCCGAAGTGTGATGACTCAGGTTTGCCCTG-3′) (SEQ ID NO 5), and mAP-1 (5′-CGCGGATCCGAAGTGTGATATCTCAGGTTTGCCCTG-3′) (SEQ ID NO 6), sense primers respectively and the P2.1 primer. The nucleotides mutated within AP-1 site are underlined. Both 187 nt products correspond to sequence −144-+43 of the human IL-8 gene. The wild type and AP-1 mutants are designated pIL-8p[wtAP-1]-Luc and pIL-8p[mutAP-1]-Luc, respectively.

IL-8 minimal promoter variants carrying mutated Oct-1/C/EBP and NF-κB sites are prepared in two PCR steps. During the first PCR with either SP3_NF-κBmut (5′-GCCCTGAGGGGATGGGCCATCAGTTGCAAATCGTTAACTTTCCTCTGACATAAT-3′) (SEQ ID NO 7), or SP3_Oct-1mut (5′-GCCCTGAGGGGATGGGCCATCAGCTACGAGTCGTGGAAT-3′) (SEQ ID NO 8),

sense primers and P2.1 antisense primer, 157 nt products are amplified carrying mutated NF-κB and Oct-1/C/EBP binding sites respectively. The nucleotides mutated within NF-κB and Oct-1 sites are underlined. During the second PCR an AP-1_Bam sense primer (5′-CGCGGATCCGAAGTGTGATGACTCAGGTTTGCCCTGAGGGGATGGGC-3′) (SEQ ID NO 9), and P2.1 antisense primer are used to reamplify both products of the first PCR reaction (100 fmol per reaction) to produce a 187 nt cDNA corresponding to sequence −144-+43 of the human IL-8 gene. The NF-κB and Oct-1/C/EBP binding site mutants are designated pIL-8p[mutNF-κB]-Luc and pIL-8p[mutOct-1]-Luc, respectively.

An IL-8 minimal promoter variant carrying a mutated CRE-like response element is prepared in three PCR steps. During the first PCR, a CREmut sense primer (5′-CAGTTGCAAATCGTGGAATTTCCTCTCGATCAATGAAAAGATG-3′) (SEQ ID NO 10),

and P2.1 antisense primer is used to produce a 137 nt product. The nucleotides mutated within CRE-like site are underlined. During the second PCR with a SP3_Oct-1wt sense primer (5′-GCCCTGAGGGGATGGGCCATCAGTTGCAAATCGTGGAAT-3′) (SEQ ID NO 11),

and P2.1 antisense primer are used to reamplify the product of the first PCR reaction (100 fmol per reaction) producing a 157 nt product corresponding to sequence −114-+43 of the human IL-8 gene. Finally, during the third PCR with AP-1_Bam sense primer and P2.1 antisense primer and the product of the second PCR reaction used as a template (100 fmol per reaction) the 5′end of the IL-8 minimal promoter variant is extended to the −144 nucleotide position of human IL-8 gene. The resulting construct used in this study is designated pIL-8p[tmutCRE_like]-Luc.

A promoter construct carrying a concatamerised CRE-like response element of IL-8 promoter is prepared by PCR with CRElike_S (5′-CGCCTGGTACCGAGCTCTG-3′) (SEQ ID NO 12), sense and CRElike_AS (5′-ACCCAAGATCTCGAGCCCG-3′) (SEQ ID NO 13), antisense primers with a template oligonucleotide (5′-CGCCTGGTACCGAGCTCTGACATAATGACATAATGACATAATGACATAATGACATAATGA CATAATTACGCGTGCTAGCCCGGGCTCGAGATCTTGGGT-3′ (SEQ ID NO 14),

(100 fmol per reaction) for the amplification. Six concatamerised copies of CRE-like response element (TGACATAA) are underlined. PCR amplification parameters are as described above. A 99 nucleotide PCR product is cleaved by Kpn I and Bgl II, gel purified and ligated into Kpn I/Bgl II digested pTAL vector (BD Biosciences) resulting in pTAL-6×[CRE_like] reporter.

DNA Preparation for High Throughput Screening

The arrayed clones discussed above are replicated to produce multiple copies for archiving. One copy is used to produce miniprep DNA using a QIAGEN BioRobot 8000 (Qiagen, Valencia, Calif.). Briefly, for each 384-well plate, 2 μl of the glycerol stock is used to inoculate a Greiner 384-deep well plate containing 100 μl Luria Broth (Gibco BRL)-8% glycerol. The Greiner plate is then covered with an airpore sheet (Qiagen), wrapped with Saran wrap, and incubated at 37° C., without shaking, for ˜22 hours. Subsequently, 5 μl of the culture is transferred from one 384-well Greiner plate into four Qiagen 96-well deep plates containing 1 ml Terrific Broth (KD Medical) (+100 ug/ml ampicillin) in each well. The four Qiagen plates are covered with airpore sheets and shaken at 250 rpm in a 37° C. incubator for ˜22 hours. Bacterial cells are pelleted by centrifugation at 4000 rpm for 15 minutes, supernatants decanted, and the plates are processed using a Qiagen BioRobot 8000 for production of DNA preparations. The protocol used is based on the manufacturer's protocol ‘QIAprep Turbo96 PB (1 to 4 plates)’, with the only modification being substituting 96-well UV-transparent-plates (Coming) as elution plates. The concentration and yield of DNA samples is determined by measuring the OD260 value on a SPECTRAmax 190 (Molecular Devices). The resulting 20,702 DNA samples are then aliquoted to produce multiple copies for archiving (at 80 pg/well in TE buffer). For assays using the 2,368 CDNA clone collection aliquots of DNA are produced in 96-well PCR plates (ABGene, Rochester, N.Y.) with 6 μl per well at 20 ng DNA/μl in OPTI-MEM I cell culture medium (Gibco BRL, Carlsbad, Calif.). DNA aliquots for screening with the 20,702 cbNA collection are produced in 384-well PCR plates with 4 μl per well at 7.5 ng plasmid/μl in OPTI-MEM. Plates are sealed with aluminum foil and stored at −20° C.

Cell Culture

Trypsinized HeLa cells (ATCC, Manassas, Va.) are resuspended in complete growth media (DMEM, Invitrogen) Containing 10% fetal bovine serum (GIBCO BRL Carlsbad, Calif. Cat# 10082-147) and 1× Antibiotic-Antimycotic reagent (GIBCO BRL Carlsbad, Calif. Cat# 15240-062) in Dulbecco's Modified Eagle Medium (D-MEM) (GIBCO BRL Carlsbad, Calif. Cat.# 10317-022) at 10⁵ cells/ml and distributed into 24 white 96-well plates (Corning, Acton, Mass.) at 75 μl per well for the 2,368 cDNA clone collection screen or into 51 white 384-well plates (Costar) at 30 μl per well for 20,702 cDNA clone collection screen using a Multidrop 384 (ThermoLabsystems). Cells are left overnight in a tissue culture incubator at 37° C. and 5% CO₂.

High Throughput Transfection Procedure.

For the 2,368 cDNA clone collection screening, 330 μg of pGL3B-IL-8ₚ-Luc reporter plasmid is resuspended in 33 ml of OptiMEM I low serum media in a 50 ml conical tube with the final amount of the reporter being 100 ng per transfection. The tube is shaken and divided into 4×8 ml aliquots. Prior to transfection, 0.8 ml of Fugene 6 transfection reagent (Roche Applied Bioscience) is added per 8 ml aliquot (4 μl of Fugene 6/μg of transfected DNA). The contents are mixed by pipeting up and down several times and distributed into 96-well clean PCR plates (ABGene) at 75 μl/well. 10 μl of [OptiMEM-reporter-Fugene 6] mix is added per well of each daughter plate containing 6 μl of prediluted cDNAs using a BiomekFX pipeting station (Beckman Coulter, Fullerton, Calif.). The last row of plate #24 is used for aliquots of pCMV-Sport6 empty vector as a negative control or pFC-MEKK an expression construct encoding sequence corresponding to M360-672 of human MEKK1 (Stratagene) as a positive control. Both plasmids are prediluted to 20 ng/μl and aliquoted 6 μl per well. After 15 minutes incubation at room temperature, 13 μl of the final mix is transferred to a 96-well HeLa culture plate. Cells are incubated for 48 hours at 37° C. in the atmosphere with 5% CO₂.

For the 20,702 cDNA clone collection screening, 1.65 mg of pGL3B-IL-8ₚ-Luc reporter plasmid is resuspended in 100 ml of OptiMEM I in 250 ml Erlenmeyer flask (Corning) with a final amount of the reporter of 50 ng per transfection. The flask is shaken and divided into 8 ml aliquots. Prior to transfection, 0.65 ml of Fugene 6 transfection reagent is added per 8 ml aliquot (3 μl of Fugene/μg of transfected DNA). The contents are mixed by pipeting up and down several times and distributed into 96-well clean PCR plates (ABGene) at 75 μl/well. 3 μl of [OptiMEM-reporter-Fugene 6] mix is added per well of each 384-well daughter plate containing 4 μl of prediluted cDNAs using a BiomekFX (Beckman). After 15 minutes incubation at room temperature, 7 μl of the mix from each well is transferred to a 384-well tissue culture plate. Cells are incubated for 48 hours at 37° C. in the atmosphere with 5% CO₂.

Luciferase Assay

48 hours post-transfection firefly luciferase activity is measured using the BrightGlo Luciferase Assay System (Promega, Madison, Wis.) following the protocol supplied by the manufacturer. Briefly 90 μl or 40 μl of freshly reconstituted Luciferase reagent is added to each well of the 96-well or 384-well tissue culture plates respectively using a Multidrop 384 (Thermo Labystems, Beverly, Mass.). After 2 minutes incubation, luminescence is read on a LUMINOSKAN Ascent Luminometer (Thermo Labsystems) with a 400 msec integration time per manufacturer's instructions.

Clone Retrieval for Hit Confirmation.

For each primary assay, Z score and fold activation against the population median were calculated according to conventional methods and deposited into an annotated searchable database. Potential hits are selected based on two criteria: (1) Z score is larger than 3.0 and (2) fold activation is greater than 10 and 5 in the 2,368 and the 20,702 cDNA clone collection screens, respectively. Clones scoring as hits in the primary assay of the 2,368 clone sub-array are retrieved from the glycerol stocks (copy 1 of the rearray plates). Hits from the primary assay of the entire 20,702 clone collection are recovered by re-transformation of DNA aliquots from the archive. Transformations are carried out in XL-10 Gold bacteria (Stratagene). Each clone is streaked out on an Luria Broth agar plate +antibiotic (100 μg/ml ampicillin) (KD Medical, Columbia, Md.), grown overnight at 37° C., and three colonies are picked from each plate, grown in deep well 96 well-plates, each well containing 995 μl Terrific Broth (KD Medical) +100 μl g/ml ampicillin. These deep well plates are covered with air pore tapes, and incubated overnight at 37° C., shaking at 300 RPM. DNA minipreps are prepared as described above. All DNA preparations are then diluted to 125 ng/μl (in wells with concentrations greater than 125 ng/μl ) and 8 μl are taken for DNA sequence confirmation. The remainder of the DNA is diluted to 25 ng/μl and 6 μl of DNA are transferred to daughter 96-well PCR plates (ABGene) and used for validation experiments using the transfection procedure described above. To normalize transfection efficiency pRL-SV40 (Promega) encoding the Renilla luciferase gene under control of the SV40 early promoter is included at 20 ng per transfection. The activity of firefly and Renilla luciferase is measured using DualGlo Luciferase Assay System (Promega) following the protocol supplied by the manufacturer. Briefly, 90 μl of freshly reconstituted Luciferase reagent is added to each well of the 96-well tissue culture plates with a Multidrop 384 and, after 15 minutes incubation, luminescence is read on a LUMINOSKAN Ascent Luminometer with a 400 msec integration time. Subsequently 90 μl of Stop-and-Glo reagent is added to each well of the 96-well tissue culture plate and, after 15 minutes incubation, luminescence is read on a LUMINOSKAN Ascent Luminometer with a 200 msec integration time. Specificity of selected clones is tested with different luciferase based promoter constructs: pCRE-Luc, p MCS-Luc (Stratagene), pTAL-Luc (BD Biosciences), pNF-kB-Luc (BD Biosciences), pIL-8ₚ-Luc, pRhoBₚ-Luc (made in house per conventional methods/BD Biosciences) and pVCAMₚ-Luc (prepared as described in lademarcom, M. F., J. J. McQuillan, G. D. Rosen, and D. C. Dean. 1992. J Biol Chem 267:16323-9.)

IL-8 Elisa Assay in HeLa cells.

HeLa cells are transfected with DNA samples selected from the group of sequence verified and confirmed hits at 100 ng per well in 96 well plates (Costar) using the protocol described above. DNA samples designated as co-activators are co-transfected at 25 ng per well. Empty vector pCMV-Sport6 is used as a negative control. 72 hours post-transfection IL-8 content is measured in the cell growth media in prediluted aliquots corresponding to 1 to 5 μl of conditioned growth media using an IL-8 Elisa kit (Sigma) following the provided protocol. As a positive control, growth media is collected from the cells transfected with empty vector and treated with IL-1β and TNFα (R&D Systems) at 5 ng/ml and 50 ng/ml respectively for 16 hours prior to collection of the growth media for the IL-8 assay.

Gene Expression Profiling with Affymetrix DNA Microarray Chips

HeLa cells are transfected with CREAP1 as described herein or expression constructs containing relA, (Ruben S M et al., Science 1991 Mar. 22;251(5000):1490-3), MAP3K11 (Hartkamp, J. et al., (1999). Cancer Res. 59, 2195-2202) or ANKRD3 (Muto, A., et al., (2002) J. Biol. Chem. 277, 31871-31876.) using Targefect F1 transfection reagent (Targeting Systems, Santee, Calif.) according to the protocol supplied with the product. Briefly, HeLa cells are used for the transfection at 70-80% of confluency in T75 tissue culture flasks (Falcon). Transfection mixes are prepared as follows: to 50 ml conical tube (Falcon) with 8 ml of Opti-MEM I 20 μg of selected plasmid DNA is added and mixed by flicking the tube. Two transfections are set up with pCMV-Sport6 empty vector. Targefect F-1 stock solution is vortexed at full speed for 20 seconds and 40 μl are added to each tube, mixed again by flicking the tube and incubated at room temperature for 30 minutes to allow formation of transfection complexes. HeLa cells are washed twice with 20 ml of Opti-MEM I medium and 12 ml of each transfection complex are added per 1 T75 flask. After 4 hrs of incubation at 37° C. 8 ml of growth media with serum is added to each flask. The media is replaced the next day. 56 hours post-transfection the media is replaced again and to one of the flasks transfected with pCMV-Sport6 plasmid TNFα (R&D Systems) is added at 50 ng/ml and the incubation is continued at 37° C. for the next 16 hours. 72 hours post-transfection cells are collected in 10 ml of TRIzol reagent (Gibco BRL) and frozen at −80° C. Total RNA is isolated according to the protocol supplied with the TRIzol reagent. Synthesis labeling of double-stranded cDNA probes, Affymetrix Gene-Chip hybridization and data analysis are done according to conventional methods (see also Eberwine, J., et al., J. Neurosci. 21, 8310-8314 and Hakak, Y., et al., (2001) Proc. Natl. Acad. Sci. U.S.A. 98, 4746-4751).

### Example 6

### Characterization of a IL8ₚ Luciferase Vector

A luciferase reporter controlled by a 1.5 kB IL-8 promoter containing fragment of human IL-8 promoter was tested for inducibility by known regulators of cytokine-mediated gene expression. pNF-κB-Luc (BD Biosciences) and pGL2-IL-8ₚ-Luc reporters were co-transfected into HEK 293 cells with expression constructs encoding known activators of the NF-κB pathway, truncated MEKK (AA 360-672) (Stratagene) and a full length TRAF6 cDNA made according to conventional methods using a proprietary clone collection. Cells co-transfected with empty pCMV-Sport6 vector were either left untreated or treated with TNFα (50 ng/ml, for 16 hours). Luciferase activity was measured 48 hours post transfection. The pNF-κB-Luc reporter was used as a positive control.

Data indicate that MEKK, TRAF6 and TNFα significantly activated the IL-8 promoter-reporter, increasing the reporter gene's activity by 16, 4.9 and 4.7 times respectively. For the high throughput functional screen of our proprietary cDNA clone collection, the IL-8 promoter sequence was subcloned into pGL3Basic vector (Promega), a derivative of the original pGL2 vector background with improved specificity and efficiency.

### Example 7

### IL-8 Promoter Based Functional Screen of the 20,000 cDNA Collection and Verification of Hit Activity

pGL3B-IL-8ₚ-Luc was co-transfected in 384-well plates with the 20,702 individual full-length cDNA clones into HeLa cells and a single reporter assay was done 48 hours post-transfection as described above. pCMV-Sport6 was co-transfected with the reporter as a negative control. Luciferase activity was measured using the BrightGlo Reporter Assay system (Promega). The absolute values of IL-8 promoter reporter activity were determined and clones scoring more than 5-fold above the pCMV-Sport6 plasmid control were identified (data not shown). To verify the identity and activity of the hits, clones were retrieved as described above, and 3 independent colonies isolated. DNA minipreps were used for sequence verification and secondary assays with the IL-8ₚ-Luc reporter (data not shown).

The individual isolates of clones producing a significant activation of the IL-8ₚ-Luc reporter in the secondary assay were tested for their ability to activate seven promoter-luciferase reporter constructs: pTAL, NF-kB-Luc, IL-8ₚ-Luc, RhoBₚ-Luc, VCAMₚ-Luc (BD Biosciences) (identical to pTAL with the addition of 4 CRE response elements).

The cDNA clones were selected based on the presence of a start codon for a predicted or characterized gene from a single 5′ end sequence of the cDNA 12,905 clones matched RefSeq genes of which 5,463 were assigned a functional annotation. The 20,704 cDNAs were co-transfected with a firefly luciferase reporter gene controlled by the IL-8 promoter (pIL-8-Luc). Sixty four cDNAs induced the reporter by greater than 5 fold. The verified active cDNAs included 1-3 copies of 28 unique genes. 22 non-redundant cDNAs were chosen for further work. The entire collection was also screened in assays for activation of a cyclic AMP Response Element (CRE) or serum response element (SRE) driven reporter. The results obtained with the 22 cDNAs in the primary screening are grouped using hierarchical clustering (Eisen) to determine if any genes appear to have related activities across the three assays. A number of genes were relatively specific for the IL-8 reporter. These included known inducers of NF-κB and were represented by relA(p65)—a subunit of NF-κB transcription factor, the TNF receptor superfamily member 1A, TNF related molecule TWEAK/TNFSF12, RIPK2 and TRAF6, respectively, a recently identified NF-κB activator ACT1 and the kinase PKK. The second group represented activators of AP-1 transcription factor sites, including multiple clones for JunD and the JNK-inducing MAP kinases MAP3K12 and MAP3K11. -C/EBPβ, known to bind directly to the IL-8 promoter NF-IL6 site was also identified. Thus, the primary screen identified a number of inducers which were predicted to activate the IL-8 gene through a number of distinct pathways.

CREAP1 was among the hits obtained. Thus, data indicate that CREAP1 is a strong activator of both the CRE-Luc and IL-8ₚ-Luc constructs. In fact, this protein of heretofore unknown function appeared not only to be the strongest activator of CRE (even stronger than the two CRE binding transactivators, CRE-BPa and CREB1 (data not shown) and confirming results disclosed in the examples provided above) but also was the strongest activator of the IL-8 gene found in these secondary assays.

### Example 8

### CREAP1 Strongly Activates a Reporter Carrying a Tandem of IL-8 Promoter-specific CRE-like Element

To determine if the strong activators also induced the endogenous IL-8 gene, the accumulation of secreted IL-8 protein from HeLa cells was measured after transfection with relA and MAP3K11 constructs as examples of NF-κB and AP-1 activators. MAP3K11 and relA induced small increases, but the combinations of both induced levels of secreted IL-8 comparable to that observed with IL-1β, one of the most potent inducers of IL-8 known. This data suggests that regulation of the endogenous IL-8 gene requires interplay of multiple signal transduction pathways.

Several cDNAs were identified whose mechanism of action is not yet clear. These included two Rho-dependendent GTP-GDP enhancing factors (Rho-GEFs), p114 and ARHGEF1, C16orf15, and thyrotroph embryonic factor 1 (TEF1), fibronectin (FN1) and nuclear receptor family member NR2F2. C16orf15 encodes a proline rich protein of unknown function, highly expressed in brain. TEF1 is a member of the basic leucine zipper transcription factors which acts directly through a TEF response element. FN1 is a matrix glycoprotein highly expressed in injured tissues and which can also induce IL-1β via AP-1-dependent mechanism. NR2F2 was a very strong activator in all assays and thus its activity appeared to be non-specific.

Several of the strongest IL-8 activators were associated with CRE-dependent gene expression. C/EBPβ, JunD, c-jun, CRE binding proteins CREB1, CRE-BPa and XBP1 were found in as potent inducers of CRE-driven reporter. A cDNA overlapping with sequences deposited for KIAA0616 and MECT1 was also identified as the CREAP1 gene discussed above. Interestingly, nothing is known about this protein except that the sequence encoding the first 44 amino acids of MECT1 are translocated onto the Mastermind-like gene MAML2 in mucoepidermoid carcinoma (Tonon et al., Nat. Genet., 33:208-213 (2003)).

The observation that many of the strongest IL-8 activators are also CRE activators or binding proteins suggests that the IL-8 promoter might contain an unrecognized CRE. This was first tested by examining the effect of elevated cAMP levels on the IL-8 promoter using plant diterpene forskolin (Sigma)—a nonspecific activator of adenylyl cyclase. Briefly, HEK 293 cells were co-transfected with either pCRE-Luc or pIL-8-Luc with empty vector or expression construct of CRE-BPa as described above using Fugene6 transfection reagent (Roche). 16 hours post transfection equal volume of growth media containing IBMX at 500 μM was added to the wells. 8 hours later forskolin was added from 50 μM stock solution prepared on growth media to the cells pre-treated with IBMX to reach 5 μM final concentration. Cells were left with forskolin for 16 hours at 37° C. Luciferase activity was determined using Dual-Glo assay kit (Promega) and normalized as described above. Data were presented as fold induction compared to untreated cells transfected with empty vector. Results indicate that forskolin weakly induced the IL-8 reporter. Co-transfection of a CRE binding protein found in the screen, CRE-BPa synergistically activated the IL-8 promoter upon forskolin. treatment.

Using standard techniques, the IL-8 promoter sequence was then examined for the presence of potential CRE sequences. A potential asymmetrical variant CRE with the sequence 5′-TGACATAA-3′ was found between −69 and −62 of the IL-8 promoter which had been previously noted as an AP-1 binding sequence but its function has not been reported (Roebuck, J. Interferon and Cytokine Res. 19:429-438 (1999)). We designated this site as “CRE-like response element”. Oligonucleotides carrying an identical DNA sequence was shown to be bound well by CREB2 and very poorly by CREB1 (Benbrook and Jones, Nucleic Acids Res., 22:1463-1469 (1994)). Interestingly, CREB2 was proposed to play a dual role as transcription activator/repressor. CREB2 bound to the “CRE-like response element” was thought to impair binding of activator proteins such as CREB and thus repress CRE-dependent transcription (Karpinski, et al. Proc. Natl. Acad. Sci. U.S.A. 89:4820-4824 (1992)). On the other hand, CREB2 was able to activate transcription of several genes working in these cases in conjunction with other transcription factors such as c-Rel, ATF-1 or the viral protein Tax (Schoch, et al. Neurochem. Int. 38:601-608 (2001)).

The mechanism of induction of the IL-8 promoter by MAP3K11 and CREAP1 was pursued. To determine if the promoter elements required activation by these genes, a series of promoter variants carrying mutations in the IL-8 CRE-like and other regulatory sites were created and tested for induction by MAP3K11, CREAP1, or relA. Results indicate that mutation of the C/EBPbinding site had no effect on activation by either protein. The NF-κB site mutation had little effect on induction by MAP3K11 or CREAP1 but eliminated induction by relA. Mutation of the AP-1 site did not significantly alter the effect of relA but severely reduced induction by MAP3K11. This is consistent with the ability of MAP3K11 to activate JNK/SAPK pathway and AP-1. Surprisingly, this mutation also significantly reduced activation by CREAP1. Mutation of the CRE-like site dramatically decreased or eliminated induction by both CREAP1 and by MAP3K11 (data not shown).

In order to determine if the “CRE-like element” was directly responsive to CREAP1 or MAP3K11, the ability of both genes to activate a minimal promoter carrying concatamerized CRE-like site (pCREL-Luc) was examined. In addition, we studied the effect of PMA known inducer of AP-1. Similar to CRE reporter (pCRE-Luc) pCREL-Luc was strongly activated by CREAP1 but neither by MAP3K11 nor by PMA treatment (data not shown). This data suggest although both CREAP1 and MAP3K11 require intact CRE-like and AP-1 sites for their activity, they induce the IL-8 promoter via different mechanisms using CRE-like or AP-1 sites respectively as their primary response elements.

We further assayed if CREAP1-induced pIL-8-Luc reporter activity is dependent on CREB. Co-expression of CREAP1 and KCREB—a dominant negative form of CREB—(BD Biosciences) led to a significant reduction of CREAP1-induced IL-8 promoter activity (data not shown). In contrast, CREAP1 activity was unaffected by co-transfection with a constitutively active form of I-KBα—a potent inhibitor of NF-KB pathway.

To determine if the interaction of CREAP1 with CRE and AP-1 binding sites is associated with the same or different domains, we constructed several variants of CREAP1 carrying deletions from N- and C-terminus using conventional methods and tested the ability of these variants to affect activation of the pIL-8-Luc reporter by either CREAP1 or MAP3K1. A mutant containing a 59 N-terminal amino acid deletion (delta59) reduced wild-type CREAP1 and greatly inhibited the MAP3K11's ability to induce expression of the IL-8 reporter (data not shown). The inhibition was specific since there was no effect of delta59 on activation by relA. Activation of an AP-1 specific reporter, pAP1 (PMA)-Luc, containing reiterated AP-1 sites, by either PMA or MAP3K11 was also blocked by delta59 (data not shown). At the same time delta59 was unable to block forskolin-stimulated pCRE-Luc reporter (data not shown). This data suggests that while CREAP1 activates expression through CREs in CREB-dependent fashion, the protein likely interacts directly or indirectly, with components essential for AP-1 activation.

### Example 9

### Gene Expression Profiling in HeLa Cells Transiently Transfected with CREAP1

To determine if CREAP1 regulates expression of authentic CREB targets, cellular gene expression was measured using DNA microarrays after overexpression of CREAP1. Briefly, HeLa cells were transiently transfected with pCMV-Sport6, CREAP1 using Targefect F1 reagent (Targeting Systems). One half of pCMV-Sport6 transfected cells were left untreated and was used as a negative control. Total RNA isolation, labeled probe preparation and DNA microchip hybridization protocol were performed as described above.

Results indicate that, interestingly, the pattern of gene expression in HeLa cells upon transfection of CREAP1 is clearly distinct from the other activators with particular enrichment of genes known to be dependent on cAMP/CREB pathway. Specifically, CREAP1 transfection induced 7 genes by greater than 10 fold (see Table 2). The other genes included well known targets of CREB and cAMP, including TSHalpha, phosphoenol pyruvate carboxykinase (PEPCK), crystallin alpha-B, and EGF-like molecule amphiregulin. CREM (another gene known to be induced upon elevation of cAMP levels) was also activated by CREAP1 to a lower extent. This set of genes was unaffected by MAP3K11 which induced PAI-2, a known target of c-Jun and AP-1 (Arts, et al., 1996 Eur. J. Biochem 241:393-402). Thus, CREAP1 is an inducer of authentic CREB target genes.

The endogenous IL-8 gene was also activated to a relatively small extent (2 to 5 fold) by each activator identified in the screen. The weak activation of the endogenous IL-8 gene compared to a strong activation observed with the artificial reporter construct is likely due to the need for activation through the multiple pathways as discussed above. We have also analyzed sets of genes differentially regulated upon CREAP1 or catalytic subunit of protein kinase A (PKA) overexpression in HEK293 cells using the hierarchical clustering algorithm. We have found that although both proteins act through CREB, the pools of genes up and down-regulated do not overlap completely. This data suggests that CREAP1 may provide an alternative to the well known phosphorylation-dependent mechanism to activate transcription.

TABLE 2
Induction of CAMP responsive genes by CREAP1.

| Gene | Affymetrix ID | Fold Activation |
|---|---|---|
| IL-8 | 1369_s_at | 2.5 |
| KIAA0467 | 41458_at | 12 |
| Exodus-1 | 40385_at | 15 |
| CAPL protein | 38088_r_at | 19 |
| amphiregulin | 34898_at | 19 |
| DKFZp566K192 | 32242_at | 32 |
| PEPCK | 33702_f_at | 32 |
| TSHa | 39352_at | 57 |

The fold increase in mRNA levels detected by Affymetrix Gene-chips for the most strongly induced genes by CREAP1 are shown. For comparison the levels of induction of the IL-8 transcripts are also shown. These were the only genes induced >10 fold by CREAP1 and all were found in duplicate experiments. Fold increase was calculated as compared to the levels of expression observed after transfection with the control pCMV-Sport6 vector.

The two most strongly induced genes by CREAP1 are known targets of cAMP, phosphoenolpyruvate carboxykinase (PEPCK or PCK1) (Roesler, W. J. Mol. Cell Endocrinol. 162:1-7 (2000)) and thyroid-stimulating hormone alpha (TSHα) ((Kim, D. S. et al. Mol Endocrinol. 8:528-36 (1994)). A third highly regulated gene, amphiregulin, was reported to be dependent on PKA for expression in some cancer lines (Bianco, C. G. et al. Clin. Cancer res. 3:439-48 (1997)) and we have identified a consensus CRE site in the proximal amphiregulin promoter that is perfectly conserved in the mouse and human genes (data not shown).

Two of the most highly induced endogenous genes by CREAP1 are not known targets of cAMP or CREB proteins. The first is CAPL; the second is the chemokine Exodus-1 (also known as CCL27, MIP-3α or LARC). Interestingly, the Exodus-1 gene is also a chemokine and is regulated in a very similar way to the IL-8 gene in that the proximal promoter is reported to contain NF-kB, AP-1 and NF-IL6/C/EBP sites. The Exodus-1 gene was also induced to a much greater extent then the endogenous IL-8 gene by CREAP1. It should also be noted that CREAP1 is a stronger inducer of Exodus-1 than TNF-α or NF-κB (data not shown). It is not known if the Exodus-1 promoter contains an unrecognized CRE or if CREAP1 might act through a variant AP-1 site as discussed. However, the activation of Exodus-1 expression by CREAP1 suggests that the Exodus-1 gene will be regulated by cAMP or by other CREB-inducing pathways.

The promoters for the CAPL, KIAA0467 and DKFZp566K192 genes have not been described. We examined the promoter of CAPL, for which no obvious CRE has been reported, for potential CREAP1-response elements. One sequence, designated CRE-like2, with the sequence 5′-TGACACAA-3′ was found in both the PEPCK promoter (nucleotides −249 and −256) and in the CAPL promoter located (nucleotides −385 and −392). The CRE-like2 element was placed upstream of a minimal promoter and tested for induction by CREAP1. This element was sufficient to mediate the induction by CREAP1 . Both the IL-8 CRE-like and the CRE-like2 sequences were modestly activated by elevated cAMP and synergistically activated by cAMP and CRE-BPa, similar to the IL-8 promoter. Thus, the CREAP1 responsive elements can be activated via cAMP pathway, however not via CREB1 since both CRE-like element found in the IL-8 promoter and CRE-like2 elements found in the CAPL and PEPCK promoters are unlikely to be recognized by CREB1.

CREAPs represent attractive targets for drug discovery. This is particularly true if the function of CREAPs is to regulate specific subsets of CREB-regulated genes by interaction of CREB with other transcription factors. Any antagonists or agonists that effect CREB directly would likely have too many effects due to the large number of CREB-responsive genes. Modulators of CREAP function on the other hand may have the ability to block specific subsets of genes, such as chemokines, e.g., IL-8 and Exodus-1 for treatment of automimmune and inflammatory disease, amphiregulin suggesting use in proliferative disorders, and PEPCK for treatment of diabetes as all of these genes are highly induced by CREAP1.

### Example 10

### Identification of CREAP2

The entire amino acid sequence for CREAP1 was used in a BLASTP search of a public NCBI database. Initially two public domain cDNAs (XM_{—}117201 and FLJ00364) were identified that have significant homology to the CREAP1 coding region. The nucleotide sequence of XM_{—}117201 was used in a BLASTN search (Altschul S. F. et al., Nucleic Acids Res. 25:3389-3402 (1997)) of a proprietary cDNA library EST database and 4 clones were identified that represent XM_{—}117201 public sequence. All 4 clones were functionally tested upon co-transfection with the CRE-Luc and IL-8p-Luc reporters initially found to be induced by CREAP using methods similar to those disclosed above. Briefly, trypsinized HeLa cells are resuspended in complete growth media at 6×10⁴ cells/ml and distributed into white 96-well plates (Costar) in the volume of 100 μl per well. Cells are left overnight in a tissue culture incubator at 37° C. and 5% CO₂. pGL3B-IL-8ₚ-Luc reporter plasmid or CRE-Luc reporter (BD Biosciences) as well as tested cDNAs are resuspended in OptiMEM I low serum media (GIBCO BRL) at 25 ng/ml. The reporter plasmids and cDNAs are then distributed into 96-well clean PCR plates (ABGene) at 4 μl/well and 3 μl/well respectively. Mixture containing Fugene 6 reagent (Roche Applied Bioscience) at 1.5 μl per transfection and pRL-SV40 (Promega) plasmid 20 ng per transfection is added in the volume of 10 μl per well of the 96-well PCR plate containing prediluted cDNAs. The content of each well is mixed by pipeting and left for 10 minutes at room temperature. 15 μl of the transfection mix from each well is transferred to a 96-well tissue culture plate. Cells are incubated for 48 hours.

The activity of firefly and Renilla luciferase is measured using the DualGlo Luciferase Assay System (Promega) following the protocol supplied by the manufacturer. Briefly, 115 μl of freshly reconstituted luciferase reagent is added to each well of the 96-well tissue culture plates with a Multidrop 384 and, after 15 minutes incubation, luminescence is read on a LUMINOSKAN Ascent Luminometer (Thermo Labsystems) with a 400 msec integration time. Subsequently, 115 μl of Stop-and-Glo reagent is added to each well of the 96-well tissue culture and, after 15 minutes incubation, luminescence is read on a LUMINOSKAN Ascent Luminometer with a 200 msec integration time. The activity of each tested cDNA is measured as a ratio of the corresponding firefly and Renilla luciferase activities.

Out of 4 clones, one clone appeared to be active. The insert of this clone was fully sequenced in one direction and appeared to encode an ORF of 586 amino acids completely overlapping with the public domain protein XP_{—}117201 predicted by XM_{—}117201 cDNA. This clone was annotated as CREAP2 and encodes a predicted protein of 693 amino acids with a start codon at nucleotide 177 and a TGA encoded stop codon at 2256. Although a search of the literature indicates there are cDNAs encoding part of CREAP2, none contain the complete sequence of human CREAP2 nor is a function for the protein provided.

The nucleotide sequence of human CREAP2 is shown below. The start codon located at nucleotide 177 and a TGA encoded stop codon at 2256 are shown in italics:

| | | |
|---|---|---|
| ANTTTTTGTACANAAAAGCAGGCTGTTACCGGTCCGGATTCCCGGGATCTAGGCTGGGGC | 60 | (SEQ ID NO 15) |
| CGGGTTCGCGGTGCTCGCTGAGGCGGCGGTGGCTACGGCTGGAGGAGCCGGGCCGAGGCC | 120 | |
| GCGGCGGAGGCCGCGGCTGGTACTGGGAGGGTGGCAGGGAGGGACGGGGAAGGAAGATGG | 180 | |
| CGACGTCGGGGGCGAACGGGCCTGGTTCGGCCACGGCCTCGGCTTCCAATCCGCGCAAAT | 240 | |
| TTAGTGAGAAGATTGCGCTGCAGAAGCAGCGTCAGGCCGAGGAGACGGCGGCCTTCGAGG | 300 | |
| AGGTGATGATGGACATCGGCTCCACCCGGTTACAGGCCCAAAAACTGCGACTGGCATACA | 360 | |
| CAAGGAGCTCTCATTATGGTGGGTCTCTGCCCAATGTTAACCAGATTGGCTCTGGCCTGG | 420 | |
| CCGAGTTCCAGAGCCCCCTCCACTCACCTTTGGATTCATCTCGGAGCACTCGGCACCATG | 480 | |
| GGCTGGTGGAACGGGTGCAGCGAGATCCTCGAAGAATGGTGTCCCCACTTCGCCGATACA | 540 | |
| CCCGCCACATTGACAGCTCTCCCTATAGTCCTGCCTACTTATCTCCTCCCCCAGAGTCTA | 600 | |
| GCTGGCGAAGGACGATGGCCTGGGGCAATTTCCCTGCAGAGAAGGGGCAGTTGTTTCGAC | 660 | |
| TACCATCTGCACTTAACAGGACAAGCTCTGACTCTGCCCTTCATACAAGTGTGATGAACC | 720 | |
| CCAGTCCCCAGGATACCTACCCAGGCCCCACACCTCCCAGCATCCTGCCCAGCCGACGTG | 780 | |
| GGGGTATTCTGGATGGTGAAATGGACCCCAAAGTACCTGCTATTGAGGAGAACTTGCTAG | 840 | |
| ATGACAAGCATTTGCTGAAGCCATGGGATGCTAAGAAGCTATCCTCATCCTCTTCCCGAC | 900 | |
| CTCGGTCCTGTGAAGTCCCTGGAATTAACATCTTTCCATCTCCTGACCAGCCTGCCAATG | 960 | |
| TGCCTGTCCTCCCACCTGCCATGAACACGGGGGGCTCCCTACCTGACCTCACCAACCTGC | 1020 | |
| ACTTTCCCCCACCACTGCCCACCCCCCTGGACCCTGAAGAGACAGCCTACCCTAGCCTGA | 1080 | |
| GTGGGGGCAACAGTACCTCCAATTTGACCCACACCATGACTCACCTGGGCATCAGCAGGG | 1140 | |
| GGCATGGGCCTGGGCCCGGCTATGATGCACCAGGACTTCATTCACCTCTCAGCCACCCAT | 1200 | |
| CCCTGCAGTCCTCCCTAAGCAATCCCAACCTCCAGGCTTCCCTGAGCAGTCCTCAGCCCC | 1260 | |
| AGCTTCAGGGCTCCCACAGCCACCCCTCTCTGCCTGCCTCCTCCTTGGCCTGCCATGTAC | 1320 | |
| TGCCCACCACCTCCCTGGGCCACCCCTCACTCAGTGCTCCGGCTCTCTCCTCCTCCTCTT | 1380 | |
| CCTCCTCCTCCACTTCATCTCCTGTTTTGGGCGCCCCCTCTTACCCTGCTTCTACCCCTG | 1440 | |
| GGGCCTCCCCCCACCACCGCCGTGTGCCCCTCAGCCCCCTGAGTTTGCTCGCGGGCCCAG | 1500 | |
| CCGACGCCAGAAGGTCCCAACAGCAGCTGCCCAAACAGTTTTCGCCAACAATGTCACCCA | 1560 | |
| CCTTGTCTTCCATCACTCAGGGCGTCCCCCTGGATACCAGTAAACTGTCCACTGACCAGC | 1620 | |
| GGTTACCCCCCTACCCATACAGCTCCCCAAGTCTGGTTCTGCCTACCCAGCCCCACACCC | 1680 | |
| CAAAGTCTCTACAGCAGCCAGGGCTGCCCTCTCAGTCTTGTTCAGTGCAGTCCTCAGGTG | 1740 | |
| GGCAGCCCCCAGGCAGGCAGTCTCATTATGGGACACCGTACCCACCTGGGCCCAGTGGGC | 1800 | |
| ATGGGCAACAGTCTTACCACCGGCCAATGAGTGACTTCAACCTGGGGAATCTGGAGCAGT | 1860 | |
| TCAGCATGGAGAGCCCATCAGCCAGCCTGGTGCTGGATCCCCCTGGCTTTTCTGAAGGGC | 1920 | |
| CTGGATTTTTAGGGGGTGAGGGGCCAATGGGTGGCCCCCAGGATCCCCACACCTTCAACC | 1980 | |
| ACCAGAACTTGACCCACTGTTCCCGCCATGGCTCAGGGCCTAACATCATCCTCACAGGGG | 2040 | |
| ACTCCTCTCCAGGTTTCTCTAAGGAGATTGCAGCAGCCCTGGCCGGAGTGCCTGGCTTTG | 2100 | |
| AGGTGTCAGCAGCTGGATTGGAGCTAGGGCTTGGGCTAGAAGATGAGCTGCGCATGGAGC | 2160 | |
| CACTGGGCCTGGAAGGGCTAAACATGCTGAGTGACCCCTGTGCCCTGCTGCCTGATCCTG | 2220 | |
| CTGTGGAGGAGTCATTCCGCAGTGACCGGCTCCAATGAGGGCACCTCATCACCATCCCTC | 2280 | |
| TTCTTGGCCCCATCCCCCACCACCATTCCTTTCCTCCCTTCCCCCTGGCAGGTAGAGACT | 2340 | |
| CTACTCTCTGTCCCCAGATCCTCTTTCTAGCATGAATGAAGGATGCCAAGAATGAGAAAA | 2400 | |
| AGCAAGGGGTTTGTCCAGGTGGCCCCTGAANTCTGCGCAAGGGATGGGCCTGNGGGGAAC | 2460 | |
| CTCANGGNNAGGGCCCAANGGCCACTTNNAANCTTTGAACCGTCNGTCTGGNANGGTCNN | 2520 | |

The predicted amino acid sequence of human CREAP2 is shown below:

| | |
|---|---|
| MATSGANGPGSATASASNPRKFSEKIALQKQRQAEETAAFEEVMMDIGSTRLQAQKLRL | (SEQ ID NO 16) |
| AYTRSSHYGGSLPNVNQIGSGLAEFQSPLHSPLDSSRSTRHHGLVERVQRDPRRMVSPL | |
| RRYTRHIDSSPYSPAYLSPPPESSWRRTMAWGNFPAEKGQLFRLPSALNRTSSDSALHT | |
| SVMNPSPQDTYPGPTPPSILPSRRGGILDGEMDPKVPAIEENLLDDKHLLKPWDAKKLS | |
| SSSSRPRSCEVPGINIFPSPDQPANVPVLPPAMNTGGSLPDLTNLHFPPPLPTPLDPEE | |
| TAYPSLSGGNSTSNLTHTMTHLGISRGHGPGPGYDAPGLHSPLSHPSLQSSLSNPNLQA | |
| SLSSPQPQLQGSHSHPSLPASSLACHVLPTTSLGHPSLSAPALSSSSSSSSTSSPVLGA | |
| PSYPASTPGASPHHRRVPLSPLSLLAGPADARRSQQQLPKQFSPTMSPTLSSITQGVPL | |
| DTSKlSTDQRLPPYPYSSPSLVLPTQPHTPKSLQQPGLPSQSCSVQSSGGQPPGRQSHY | |
| GTPYPPGPSGHGQQSYHRPMSDFNLGNLEQFSMESPSASLVLDPPGFSEGPGFLGGEGP | |
| MGGPQDPHTFNHQNLTHCSRHGSGPNIILTGDSSPGFSKEIAAALAGVPGFEVSAAGLE | |
| LGLGLEDELRNEPLGLEGLNMLSDPCALLPDPAVEESFRSDLQ | |

### Example 11

### Identification of CREAP3

Using methodologies similar to those disclosed above, a clone was found in our proprietary cDNA library EST database by comparison with the sequence of the public domain clone, cDNA FLJ00364. The predicted protein encoded by FLJ00364 lacked an initiator ATG and had an N-terminal sequence with no homology to CREAP1. Comparison of the public domain clone sequence with a similar clone in our database revealed that our proprietary clone sequence contained an extra C at the sequence CCGTCATTTCACCAAGC (SEQ ID NO 17) where the extra C is designated by an underline. This extra C was confirmed by comparison with the genomic sequence. This change resulted in the elimination of the first 63 amino acids predicted by the FLJ00364 cDNA and substituted an inframe altemate 81 amino acids starting at amino acid sequence EETRAFE (SEQ ID NO 18) highly conserved with the CREAP1 predicted protein sequence, E²³ETAAFE (SEQ ID NO 19).

A series of three sequential Polymerase Chain Reactions (PCR) was performed to the complete ORF of the proprietary clone. PCR amplification cycles consisted of: 2 min at 94° C., 23×[15 s at 94° C., 30 s at 68° C. and 15 s at 72° C.]and 2 min at 72° C. Advantage 2 DNA polymerase (BD Biosciences) was used for all the amplification steps. All three PCR products were amplified with a common sense primer KIAAhS3_R1 with the nucleotide sequence (5′-CCGGAATTCGCCATGGCCGCCTCGCCGGGCTCGGG-3′) (SEQ ID NO 20) corresponding to the start of the ORF. An EcoRI restriction site was included in the 5 prime end sequence of the primer using conventional methods. For the initial PCR, human genomic DNA (BD Biosciences) was used as a template (2 mg per reaction), and antisense primers KIAAhAS2 (5′-CCGCGACAGGGTGAGGTCGGTCATGAGCTGCTCGAAGGCCCGCG-3′) (SEQ ID NO 21). 142 nt PCR product was extracted with phenol-chloroform mixture and precipitated by isopropanol. The precipitate was washed with ice-cold 70% ethanol and resuspended in TE buffer. 5 ng of the product was used as a template in the second PCR with KIAAhS3_R1 sense and KIAAhAS3 (5′-GAAGCTTCTGAAATTGAACCCGCGACAGGGTGAGGTCGGTCATG-3′) (SEQ ID NO 22) antisense primers. A 161 nt PCR product was processed similar to the original PCR product and 5 ng of resuspended DNA was used as a template in the final PCR with KIAAhS3_R1 sense and KIAAhAS4 (5′-GGTAAGGATCCTCCATGGTACTGTGTAAGGCGCAGTTGCTGAAGCTTCTGAAATTGAA CCCG-3′) SEQ ID NO 23) antisense primers. All primers were obtained from SIGMA-Aldrich Corp., (Saint Louis, Mo., USA) or made according to conventional methods.

A 202 nt product was gel purified and cut with EcoRI and BamHI and inserted into a EcoRI/BamHI digested plasmid of the proprietary clone. 16 individual clones of reconstructed full-length FLJ00364 cDNA were sequence verified and functionally tested with CRE-Luc and IL-8p-Luc reporters as described above. Clone #5 free of PCR-introduced mismatches and strongly activating both reporters was used for DNA and protein alignments and has been annotated as CREAP3.

The nucleotide sequence of CREAP3 is provided below. The start codon at nucleotide 46 and a TGA stop codon at 1905 are shown in italics. Note that the C at residue 288 shown in bold underline has been added due to comparison with the genomic sequence and proprietary clone sequence. The underlined CGAGG sequence indicates the 5′-end of the proprietary clone. The nucleotide sequence upstream of this sequence was amplified by PCR using genomic DNA as a template and inserted back into the proprietary clone as described above.

Nucleotide sequence of CREAP3:

| | | |
|---|---|---|
| NTTTTTTGTACANAAAAGCAGGCTGTTACCGGTCCGGAATTCGCCATGGCCGCCTCGCCG | 60 | (SEQ ID NO 24) |
| GGCTCGGGCAGCGCCAACCCGCGGAAGTTCAGTGAGAAGATCGCGCTGCACACGCAGAGA | 120 | |
| CAGGCCGAGGAGACGCGGGCCTTCGAGCAGCTCATGACCGACCTCACCCTGTCGCGGGTT | 180 | |
| CAATTTCAGAAGCTTCAGCAACTGCGCCTTACACAGTACCATGGAGGATCCTTACCAAAT | 240 | |
| GTGAGCCAGCTGCGGAGCAATGCGTCAGAGTTTCAGCCGTCATTTCACCAAGCTGATAAT | 300 | |
| GTTCGGGGAACCCGCCATCACGGGCTGGTGGAGAGGCCATCCAGGAACCGCTTCCACCCC | 360 | |
| CTCCACCGAAGGTCTGGGGACAAGCCAGGGCGACAATTTGATGGTAGTGCTTTTGGAGCC | 420 | |
| AATTATTCCTCACAGCCTCTGGATGAGAGTTGGCCAAGGCAGCAGCCTCCTTGGAAAGAC | 480 | |
| GAAAAGCATCCTGGGTTCAGGCTGACATCTGCACTTAACAGGACCAATTCTGATTCTGCT | 540 | |
| CTTCACACGAGTGCTCTGAGTACCAAGCCCCAGGACCCCTATGGAGGAGGGGGCCAGTCG | 600 | |
| GCCTGGCCTGCCCCATACATGGGGTTTTGTGATGGTGAGAATAATGGACATGGGGAAGTA | 660 | |
| GCATCTTTCCCTGGCCCATTGAAAGAAGAGAATCTGTTAAATGTTCCTAAGCCACTGCCA | 720 | |
| AAACAACTGTGGGAGACCAAGGAGATTCAGTCCCTGTCAGGACGCCCTCGATCCTGTGAT | 780 | |
| GTTGGAGGTGGCAATGCTTTTCCACATAATGGTCAAAACCTAGGCCTCTCACCCTTCTTG | 840 | |
| GGGACTTTGAACACTGGAGGGTCATTGCCAGATCTAACCAACCTCCACTACTCGACACCC | 900 | |
| CTGCCAGCCTCCCTGGACACCACCGACCACCACTTTGGCAGTATGAGTGTGGGGAATAGT | 960 | |
| GTGAACAACATCCCAGCTGCTATGACCCACCTGGGTATAAGAAGCTCCTCTGGTCTCCAG | 1020 | |
| AGTTCTCGGAGTAACCCCTCCATCCAAGCCACGCTCAATAAGACTGTGCTTTCCTCTTCC | 1080 | |
| TTAAATAACCACCCACAGACATCTGTTCCCAACGCATCTGCTCTTCACCCTTCGCTCCGT | 1140 | |
| CTGTTTTCCCTTAGCAACCCATCTCTTTCCACCACAAACCTGAGCGGCCCGTCTCGCCGT | 1200 | |
| CGGCAGCCTCCCGTCAGCCCTCTCACGCTTTCTCCTGGCCCTGAAGCACATCAAGGTTTC | 1260 | |
| AGCAGACAGCTGTCTTCAACCAGCCCACTGGCCCCATATCCTACCTCCCAGATGGTGTCC | 1320 | |
| TCAGACCGAAGCCAACTTTCCTTTCTGCCCACAGAAGCTCAAGCCCAGGTGTCGCCGCCA | 1380 | |
| CCCCCTTACCCTGCACCCCAGGAGCTCACCCAGCCCCTCCTGCAGCAGCCCCGCGCCCCT | 1440 | |
| GAGGCCCCTGCCCAGCAGCCCCAGGCAGCCTCCTCACTGCCACAGTCAGACTTTCAGCTT | 1500 | |
| CTCCCGGCCCAGGGCTCATCTTTGACCAACTTCTTCCCAGATGTGGGTTTTGACCAGCAG | 1560 | |
| TCCATGAGGCCAGGCCCTGCCTTTCCTCAACAGGTGCCTCTGGTGCAACAAGGTTCCCGA | 1620 | |
| GAACTGCAGGACTCTTTTCATTTGAGACCAAGCCCGTATTCCAACTGCGGGAGTCTCCCG | 1680 | |
| AACACCATCCTGCCAGAAGACTCCAGCACCAGCCTGTTCAAAGACCTCAACAGTGCGCTG | 1740 | |
| GCAGGCCTGCCTGAGGTCAGCCTGAACGTGGACACTCCATTTCCACTGGAAGAGGAGCTG | 1800 | |
| CAGATTGAACCCCTGAGCCTGGATGGACTCAACATGTTAAGTGACTCCAGCATGGGCCTG | 1860 | |
| CTGGACCCCTCTGTTGAAGAGACGTTTCGAGCTGACAGACTGTGAACAGAAGGCAGTGGA | 1920 | |
| ACAGAAGAATGTTTTTCTGCAACAGCCAAAATAGAATGGAATAGAATGAAGCCAGCTGAT | 1980 | |
| ACCACGGGCTTTCGTTATCTTGACATAGAAGGAAGCAGTGCCACGGCTCCAGGGTTTCAG | 2040 | |
| ATGAGATCCCATCTCAGACACTGTGGCTTCCTCCAGATCACACAGCTTTGTACTGCCTCT | 2100 | |
| CCCGCCTGTGGCCAAAGTCGTGTTGCAGCAGGCAGGCTGCTTGGAGCTTCCCATGAACTG | 2160 | |
| GAAAGCTCACCTCCACTGCATCTTTTTACTGGCCATCCAGTCAGCCGATGTGTAAGAGTA | 2220 | |
| GGAAATACTGTGTCACTGGAGGCCCTCCGTAGCATTGGG | 2259 | |

The CREAP3 cDNA encodes a predicted protein of 619 amino acids as shown below with a start codon at nucleotide 46 and a TGA encoded stop codon at 1905. The alternative correct sequence of amino acids encoded by CREAP3 different from the sequence predicted by public clone FLJ00364 is underlined. The glutamic acid and alanine at amino acid positions 551 and 616 are shown in bold.

(SEQ ID NO 25)

MAASPGSGSANPRKFSEKIALHTQRQAEETRAFEQLMTDLTLSRVQFQKLQ
QLRLTQYHGGSLPNVSQLRSNASEFQPSFHQADNVRGTRHHGLVERPSRNR
FHPLHRRSGDKPGRQFDGSAFGANYSSQPLDESWPRQQPPWKDEKHPGFRL
TSALNRTNSDSALHTSALSTKPQDPYGGGGQSAWPAPYMGFCDGENNGHGE
VASFPGPLKEENLLNVPKPLPKQLWETKEIQSLSGRPRSCDVGGGNAFPHN
GQNLGLSPFLGTLNTGGSLPDLTNLHYSTPLPASLDTTDHHFGSMSVGNSV
NNIPAAMTHLGIRSSSGLQSSRSNPSIQATLNKTVLSSSLNNHPQTSVPNA
SALHPSLRLFSLSNPSLSTTNLSGPSRRRQPPVSPLTLSPGPEAHQGFSRQ
LSSTSPLAPYPTSQMVSSDRSQLSFLPTEAQAQVSPPPPYPAPQELTQPLL
QQPRAPEAPAQQPQAASSLPQSDFQLLPAQGSSLTNFFPDVGFDQQSMRPG
PAFPQQVPLVQQGSRELQDSFHLRPSPYSNCGSLPNTILPEDSSTSLFKDL
NSALAGLPEVSLNVDTPFPLEEELQIEPLSLDGLNMLSDSSMGLLDPSVEE
TFRADRL

Due to the extra C described above at position 288, the first 81 amino acids are different between polypeptides predicted by FLJ00364 and the corrected proprietary clone.

We believe that the amino acid sequence encoded by CREAP 3 shown is correct because it shows extensive homology with CREAP 1 and CREAP 2. Briefly, CREAP gene family sequences were compared using ClustalW. Amino acid identities were determined with Align, version 2.0 (Myers E. W. and Miller W., Bull. Math Biol 51: 5-37 (1989)) and the Blosum 50 scoring matrix (CITE). Alignment with genomic sequences was done using BlastN and the Celera CHD database (Celera Genomics, Rockville, Md.) and searched using the masked consensus human sequence. (file: CHGD_masked_assembly_{—}500k-i).

The amino acid sequences predicted by the proprietary clone and the FLJ00364 cDNAs are different in two other areas. The proprietary clone contains an additional GAA triplet resulting in an addition of glutamic acid at amino acid position 551 as shown above. Finally, a single nucleotide A/G change in the CREAP3 cDNA results in a threonine/alanine change at amino acid position 616 as shown above.

### Example 12

### Identification of CREAP Genes from other Species

Identification of a Drosophila

CREAP Gene, dCREAP:

BLASTP searches of Genebank protein and DNA sequence databases performed according to conventional methods with both CREAP1 and CREAP3 coding regions identified a single predicted Drosophila gene, CG6064. This gene has been designated dCREAP and its amino acid sequence is shown below. This sequence was found as a predicted gene of unknown function from sequencing of the D. melanogaster genome, GenBank entry |17293954|gb|AAF49313.1| CG6064-PA [Drosophila melanogaster] (Adams et al., Science 287(5461):2185-2195 (2000)). The dCREAP gene CG6064 contains no inserts and predicts a protein of 797 amino acids, somewhat larger than the human CREAPs.

dCREAP DNA sequence

| | |
|---|---|
| ATGGCCAATCCGCGCAAGTTCAGCGAGAAGATCGCTCTGCAGAAGCAGAAGCAGGCGGAGGGCACAGCGG | (SEQ ID NO 26) |
| AATTCGAGCGGATCATGAAGGAGGTGTATGCCACGAAGAGGGATGAGCCGCCTGCGAATCAGAAGATCCT | |
| AGACGGCCTTGTCGGCGGTCAGGAGGTAAGCCAATCCTCGCCAGGCGCAGGCAATGGGACGGGCGGAGGT | |
| GGCAGTGGTTCCGGCAGTGGAGCCAGCGGCGGAGGAGCCTCACCAGATGGCCTGGGAGGCGGCGGTGGTT | |
| CTCCGACGGCTTATCGAGAATCCCGAGGGCGCAGCGTAGGTGTGGGTCCCATGCGAAGACCGTCGGAGCG | |
| CAAGCAGGATCGTTCGCCCTACGGCAGCAGCAGTACGCAACAAACCTTAGACAACGGCCAGCTAAATCCG | |
| CATCTTCTTGGTCCACCTACGGCGGAGAGTTTGTGGCGGCGGTCCAGCTCCGATTCGGCGCTGCACCAAA | |
| GTGCGCTGGTGGCGGGCTTCAATAGCGACGTGAACTCGATGGGCGCCAACTATCAGCAGCAGCAACATCA | |
| GCAACAACAGCAACCGGGCCAGCCAAGATCTCACTCGCCGCACCATGGTATAAACAGGACCATGAGTCCG | |
| CAGGCGCAACGGAGGAAGTCGCCGCTACTGCAGCCCCATCAGCTGCAGTTGCAGCAACTGCAACAGCAGC | |
| AGCAACAGATGCAACATCAGCATCAGCTGCACCAGCAGCTCCAAATGCAGCAGCTGCAACAGCACCAGCA | |
| GCAACACCAGCAGCAGCAGCAACAACAGAACACGCCATACAACAACGCCAAATTCACGAATCCTGTGTTC | |
| CGGCCGCTGCAGGATCAGGTCAACTTTGCCAACACCGGCTCCCTGCCCGATCTCACGGCCCTTCAAAACT | |
| ATGGACCCCAGCAGCAGCAGCAGCAATCCCAGCAACAGCCGTCGCAGCAACAACAGCAGTTGCAGCAAAC | |
| CCTGTCGCCAGTCATGTCTCCGCACAATCACCGCCGCGAACGGGATCAGTCGCCCAGTCCGTTTAGTCCG | |
| GCGGGTGGAGGAGGGGGAGCAGGTCCCGGGTCGCCCTATCAGCAGCAACAGCACTCGCCCACCGGAAACA | |
| CGCAACAGCAGCAGCAGCAGCACCAACAGCCCAGCAACTCGCCGCACCTGTCCTTTACCAATCTGGCCAC | |
| CACGCAGGCAGCTGTTACCACATTTAACCCGCTCCCCACGCTGGGTCCGCACAATGCCACCGACTACCGC | |
| CAGCCACCGAATCCTCCTAGTCCACGCTCTTCGCCCGGCTTGCTGAGCAGCGTATCGGCCACGGATCTGC | |
| ACTCCAGTGCACCGGCCAGTCCCATACGCCAGCAGCAACAGGCCCATCAGCAGCAACAGCAGCAGCAACA | |
| GGCGCAGCAACAACAGCAACAGTTTGATAACTCCTACAACAGTCTGAATACCTCGTTTCACAATCAGTTT | |
| GAGATTTTCTCGCTGGGCGACAGCAATTCCTCGCCGGAACAGCAGGGCTTTGCAAATAATTTCGTGGCCC | |
| TCGACTTTGACGACCTGAGTGGCGGCGGAGGTGGTGGCCCAAGCGGGGGCGGCGGCAGCAATGGAGGAGG | |
| TCTGACCAACGGTTACAACAAGCCGGAGATGTTGGACTTCAGCGAGCTGAGCGGCAGCCCGGAGGCGAGT | |
| GGGAACAACAACCACATGCGGCGAGGAGTGAGCAACCTGAACAACAACGGGTTGAGCAATGGTGTGGTGG | |
| GATCCACGCACAACGGCAGCACAAATCTAAATGGAGCGGGAAACAACAATAGCAGTAGTGGAGGTGGCAC | |
| GGCGCAGGATCCTTTGGGAATAACCACTTCGCCTGTGCCCTCACCCTTGGGCTGCCCCAGTTCACCGCTG | |
| CCGATACCGATTCCGATGTCGGCGCAAAGCTCGCCACAGCAGCAGCACCACCATCATCAGCAGCAGCAAC | |
| AACAGCATCATCAGCAGCAACACCATCAGCAGCAGCAATTATCATTATCTCTGCACCATTCGCCGCATCA | |
| TTCGCCAATGCATTCGCCGCACCATGGGAATTCACCGCTTTCAAGCAGCTCGCCAGTGAGTCACAATGCC | |
| TGCTCCAACTCCAACGTGGTGATGAACCACCAGCAGCAGCAGCAACAACATCACCACCAGCAACACCATC | |
| ATCAGGGCTCCTCGCAAAGTCACACGCCGACCACAGCGAATATACCCTCTATTATCTTTAGTGATTACTC | |
| CTCCAACGCGGATTATACCAGGGAGATCTTCGACTCCCTCGATCTGGATCTGGGACAGATGGACGTAGCC | |
| GGTTTGCAGATGCTGTCCGACCAGAACCCCATCATGATCGCCGATCCCAACATCGAGGATAGTTTTCGAC | |
| GCGACCTCAACTGATACTATGAGGAGGCTGTTGCGGCCATTGAGAGCGGAGTGCTGCTGGAGGAGGACTA | |
| CCAGGCGCTGCTCGGATCAGAGGCGCTGGCGGATGAACAGGTGGTCACAGTCGAGGCCGCCGGAGCCGCA | |
| GCAGCAGTAGTAACAGTTGAAGAGGCAGCCACAGTTAGCGAGAAGGACAAAAAAGATTTGGAAGTTGTGG | |
| AACTTCTGGTGTCCGGTGTTATGGATGACCTGGTGGACTCCAGTGACCTGGACGAGGAAGTGCGCAATTT | |
| CTTTTTTTAGGCAGCCAGCAAGTCATTTTTGTCGTTAACACAACTGATGGAATTTTCGTTTTTAACACAG | |
| ATGAGGAAGTGAATTACGTTTTTTAAACGCATTCACTTGCCATTTCTCGATTAAATGCCATATTACTTAA | |
| GCTCAGGATTTACAAGCTTAATGCGAATTAAGTTAATTTCGGAAATGCTGACGAGAGTGATTGCAAAGTT | |
| CAAAATTGATACAAATTCACTTCCGCAAATTCATGCTGAAACTGAAAGTTTTCTAACAGTCCTCAATATT | |
| GTTATCTCGTTATCGTCCGTGCTTTCGTAGCTAGCTCCTACAACAAAAATAC | |

The predicted amino acid sequence for dCREAP is shown below:

| | |
|---|---|
| MANPRKFSEKIALQKQKQAEGTAEFERIMKEVYATKRDEPPANQKILDGLVGGQEVSQSSPGAGNGTG | (SEQ ID NO 27) |
| GGGSGSGSGASGGGASPDGLGGGGGSPTAYRESRGRSVGVGPMRRPSERKQDRSPYGSSSTQQTLDNG | |
| QLNPHLLGPPTAESLWRRSSSDSALHQSALVAGFNSDVNSMGANYQQQQHQQQQQPGQPRSHSPHHGI | |
| NRTMSPQAQRRKSPLLQPHQLQLQQLQQQQQQMQHQHQLHQQLQMQQLQQHQQQHQQQQQQQNTPYNN | |
| AKFTNPVFRPLQDQVNFANTGSLPDLTALQNYGPQQQQQQSQQQPSQQQQQLQQTLSPVMSPHNHRRE | |
| RDQSPSPFSPAGGGGGAGPGSPYQQQQHSPTGNTQQQQQQHQQPSNSPHLSFTNLATTQAAVTTFNPL | |
| PTLGPHNATDYRQPPNPPSPRSSPGLLSSVSATDLHSSAPASPIRQQQQAHQQQQQQQQAQQQQQQFD | |
| NSYNSLNTSFHNQFEIFSLGDSNSSPEQQGFANNFVALDFDDLSGGGGGGPSGGGGSNGGGLTNGYNK | |
| PEMLDFSELSGSPEASGNNNHMRRGVSNLNNNGLSNGVVGSTHNGSTNLNGAGNNNSSSGGGTAQDPL | |
| GITTSPVPSPLGCPSSPLPIPIPMSAQSSPQQQHHHHQQQQQQHHQQQHHQQQQLSLSLHHSPHHSPM | |
| HSPHHGNSPLSSSSPVSHNACSNSNVVMNHQQQQQQHHHQQHHHQGSSQSHTPTTANIPSIIFSDYSS | |
| NADYTREIFDSLDLDLGQMDVAGLQMLSDQNPIMIADPNIEDSFRRDLN | |

The activity of dCREAP is analyzed according to the following method:

The 2.3 kb cDNA encoding dCREAP open reading frame was amplified by PCR using sense (SEQ ID 37) and antisense (SEQ ID 38) primers.

Sense primer used to amplify dCREAP ORF cDNA: (the Drosophila Kozak sequence CAAC is underlined)

| | |
|---|---|
| CAACATGGCCAATCCGCGCAAGTTCAGCGAG | (SEQ ID 37) |

Antisense primer used to amplify dCREAP ORF cDNA:

| | |
|---|---|
| TCAGTTGAGGTCGCGTCGAAAACTATCCTC | (SEQ ID 38) |

The amplified product was inserted into the Drosophila P-element transformation vector, pUAST (Brand and Perrimon, Development 118:401-415 (1993)). The final construct pUAS-dCREAP was used for transfection experiments in Drosophila melanogaster Schneider cells (S2). A firefly luciferase reporter was created which contained 4 copies of the drosophila CRE enhancer element (SEQ ID 39) (Eresh, S. et. Al. EMBO J. 16:2014-2022 (1997)) followed by hsp70 minimal promoter.

Oligonucleotide sequence containing 4 copies of the Drosophila CRE. The sequence of CRE elements are underlined:

| | |
|---|---|
| GGAGCCTGGCGTCAGAG AGCCTGGCGTCAGAG AGCCT | (SEQ ID 39) |
| GGCGTCAGAGAGCCTGGCGTCAGAG | |

The S2 cells were transfected in 6 well plates (Costar) by the CaPO₄ method (Bunch, T. and Goldstein, L. Nucleic Acids Res. 17:9761-9782 (1989)). A total of 25 ug of DNA was transfected into a 6-well dish containing 4 mls of cells (˜1×10⁶ cells/ml). The transfection mix was removed after 18 hr and the luciferase assays were performed 48 hrs later. The UAS-transgenes were activated by co-transfection with the Actin promoter-Gal4 plasmid provided by Dr. Norbert Perrimon. The transfection efficiency was normalized co-transfection with hsp^{mln} Renilla luciferase driven by minimal heat shock promoter (made according to conventional methods). Luciferase activity was measured using the Dual-luciferase assay kit (Promega). As a negative control S2 cells were co-transfected with CRE-hsp-Luc reporter and empty pUAST vector. Data were calculated as fold induction compared to the reporter gene's activity measured in the cells designated as negative control.

Results indicate (see Table 3) that, like human CREAPS, dCREAP can also regulate CREs in Drosophila, as it has potently induced the activity of CRE-hsp-Luc reporter when CRE elements are present.

TABLE 3
dCREAP potently induces the activity of CRE-hsp-Luc reporter.

| | Fold | |
|---|---|---|
| | activation | STDEV |
| pUAST/CRE-hsp-Luc | 1.02 | 0.28 |
| dCREAP/hsp-Luc | 0.96 | 0.15 |
| dCREAP/CRE-hsp-Luc | 136.04 | 37.13 |

S2 cell were co-transfected with empty pUAST vector or pUAST-dCREAP construct (dCREAP) and either hsp-Luc reporter or hsp-Luc reporter carrying 4 copies of Drosophila CRE (CRE-hsp-Luc).
Luciferase activity was assayed 48 hours post-transfection.

Identification of a Mouse CREAP1 (mCREAP1) Gene:

A mouse CREAP1 protein was also identified using conventional methods. Briefly, mCREAP1 cDNA was assembled in the following order:
Nucleotides 1-483 were taken from mouse EST BY752080 (here and below GenBank Accession numbers);
Nucleotides 484-891 were taken from mouse EST BM950955;
Nucleotides 892-909 were taken from mouse genomic DNA sequence Celera clone
Nucleotides 910-981 were taken from mouse EST CA326891.
Nucleotides 982-1610 were taken from mouse EST BM935820.
Nucleotides 1611-2416 were taken from mouse EST BI453510.

Resulting nucleotide sequence of mCREAP1:

| | |
|---|---|
| GGGACGAAGAGTAGGAGTAGGAGGAGGCGGCGAGAAGATGGCGACTTCGAACAATCCGCGGAAATTTA | (SEQ ID NO. 28) |
| GCGAGAAGATCGCACTGCACAACCAGAAGCAGGCGGAGGAGACGGCGGCCTTCGAGGAGGTCATGAAG | |
| GACCTGAGCCTGACGCGGGCCGCGCGGCTTCAGCTGCAGAAGTCCCAGTACCTGCAGCTGGGCCCCAG | |
| CCGTGGCCAGTACTACGGTGGGTCCCTGCCCAACGTGAACCAGATTGGAAGCAGCAGCGTGGACCTGG | |
| CCTTCCAGACCCCATTTCAGTCCTCAGGCCTGGACACGAGTCGGACCACACGACATCATGGGCTTGTG | |
| GACAGAGTATATCGTGAGCGTGGCAGACTTGGCTCCCCGCACCGTCGACCCCTGTCAGTAGACAAGCA | |
| TGGGCGACAGGCTGACAGCTGCCCCTATGGCACCGTGTACCTCTCGCCTCCTGCGGACACCAGCTGGA | |
| GGAGGACCAACTCTGACTCTGCCCTGCACCAGAGCACAATGACACCCAGCCAGGCAGAGTCCTTCACA | |
| GGCGGGTCCCAGGATGCGCACCAGAAGAGAGTCTTACTGCTAACTGTCCCAGGAATGGAGGACACCGG | |
| GGCTGAGACAGACAAGACCCTTTCTAAGCAGTCATGGGACTCAAAGAAGGCGGGTTCCAGGCCCAAGT | |
| CCTGTGAGGTCCCCGGAATCAACATCTTTCCGTCTGCAGACCAGGAGAACACAACAGCCCTGATCCCT | |
| GCCACCCACAACACAGGGGGCTCCCTTCCTGACCTCACCAACATCCACTTCGCCTCCCCACTCCCGAC | |
| ACCACTGGACCCTGAGGAGCCTCCGTTCCCTGCTCTCACCAGCTCCAGCAGCACCGGCAGCCTTGCAC | |
| ATCTGGGCGTTGGCGGCGCAGGCGGTATGAACACCCCCAGCTCTTCTCCACAGCACCGGCCAGCAGTC | |
| GTCAGCCCCCTGTCCCTGAGCACAGAGGCCAGGCGGCAGCAGGCCCAGCAGGTGTCACCCACCCTGTC | |
| TCCGTTGTCACCCATCACTCAGGCCGTGGCTATGGATGCCCTGTCCTTGGAGCAGCAGCTGCCCTATG | |
| CCTTCTTCACCCAGACTGGCTCCCAGCAGCCTCCCCCACAGCCCCAGCCACCGCCTCCACCTCCACCG | |
| GTATCCCAGCAGCAGCCACCACCTCCACAGGTGTCTGTGGGCCTCCCCCAGGGTGGTCCACTGCTGCC | |
| CAGTGCCAGCCTGACTCGGGGGCCCCAGCTGCCACCACTCTCAGTTACTGTACCATCCACTCTTCCCC | |
| AGTCCCCTACAGAGAACCCAGGCCAGTCACCAATGGGGATCGATGCCACTTCGGCACCAGCTCTGCAG | |
| TACCGCACGAGTGCAGGGTCACCTGCCACCCAGTCTCCCACCTCTCCGGTCTCCAACCAAGGCTTCTC | |
| CCCTGGAAGCTCCCCACAGCACACGTCCACCCTGGGCAGCGTGTTTGGGGATGCGTACTATGAGCAGC | |
| AGATGACAGCCAGGCAGGCCAATGCTCTGTCNCGCCAGCTGGAGCAGTTCAACATGATGGAGAACGCC | |
| ATCAGCTCCAGCAGCCTATACAACCCGGGCTCCACACTCAACTATTCCCAGGCTGCCATGATGGGTCT | |
| GAGCGGGAGCCACGGGGGCCTACAGGACCCGCAGCAGCTCGGCTACACAGGCCACGGTGGAATCCCCA | |
| ACATCATCCTCACGGTGACAGGAGAGTCACCACCGAGCCTCTCTAAGGAACTGAGCAGCACACTGGCA | |
| GGAGTCAGTGATGTCAGCTTTGATTCGGACCATCAGTTTCCACTGGACGAGCTGAAGATTGACCCTCT | |
| GACCCTGGACGGACTCCATATGTTGAATGACCCAGACATGGTTTTAGCCGACCCAGCCACCGAGGACA | |
| CCTTCCGAATGGACCGCCTGTGAGTGGCTGTGCCCACCAGCCGCCGCTGGTCAGTCTCCAACGGCGCT | |
| GCCCCAAACCTGGGGACGGCAATGGCGTCCCCCTTTGCCAACGGCCAAGCTTGTGGTTCTGAGCTTGC | |
| AATGCTGCCCAGTGCCCCTGCCAGCCCCCCGCCACCCCGGTCGTTCACCTCCCATGATGCCTGGCGTG | |
| CGTGAGGCCGCTGTGTACTAGGCTGGCTATCTGTCTGTCCATCCATCTACCTGGGGTCAGGCTGATGG | |
| CCGAGGCTGTGAGTGCCTGGCCCCCATGGATGTTCCCCGTGCTCGCTCCCTCACCCCTCACTGGGGAT | |
| GTGAGAGCCCTCATCAGATACCCAAAGTGTCACTCACTTCCAGCATGTGCTGTGCAACGGAGGGCCGG | |
| GGCGTGGGTGTGGAGCGCCCAGAGGCTTAGGTGCGCCATCCATTCGACTGTTGTCAGCTGTCACTGCC | |
| TTCCTCCATCCTGTCCCCCGTCCCACCGCCATCCCT | |

The open reading frame encoding the protein sequence of mCREAP1 is encoded by nucleotides 25-1914.

Protein sequence of mCREAP1:

| | |
|---|---|
| MATSNNPRKFSEKIALHNQKQAEETAAFEEVMKDLSLTRAARLQLQKSQYLQLGPSRGQYYGGSLPNV | (SEQ ID NO:29) |
| NQIGSSSVDLAFQTPFQSSGLDTSRTTRHHGLVDRVYRERGRLGSPHRRPLSVDKHGRQADSCPYGTV | |
| YLSPPADTSWRRTNSDSALHQSTMTPSQAESFTGGSQDAHQKRVLLLTVPGMEDTGAETDKTLSKQSW | |
| DSKKAGSRPKSCEVPGINIFPSADQENTTALIPATHNTGGSLPDLTNIHFASPLPTPLDPEEPPFPAL | |
| TSSSSTGSLAHLGVGGAGGMNTPSSSPQHRPAVVSPLSLSTEARRQQAQQVSPTLSPLSPITQAVAMD | |
| ALSLEQQLPYAFFTQTGSQQPPPQPQPPPPPPPVSQQQPPPPQVSVGLPQGGPLLPSASLTRGPQLPP | |
| LSVTVPSTLPQSPTENPGQSPMGIDATSAPALQYRTSAGSPATQSPTSPVSNQGFSPGSSPQHTSTLG | |
| SVFGDAYYEQQMTARQANALSRQLEQFNMMENAISSSSLYNPGSTLNYSQAAMMGLSGSHGGLQDPQQ | |
| LGYTGHGGIPNIILTVTGESPPSLSKELSSTLAGVSDVSFDSDHQFPLDELKIDPLTLDGLHMLNDPD | |
| MVLADPATEDTFRMDRL | |

Identification of a Fugu CREAP1:

A CREAP1 was identified in Fugu rubripres. The sequence was identified by aligning the human CREAP1 protein sequence against the fugu genome (version 3) using TBLASTN. Highly homologous regions were retrieved from the alignment. The retrieved sequence was further hand-edited.

Fugu CREAP1 amino acid sequence:

| | |
|---|---|
| MASSNNPRKFSEKIALHNQKQAEETAAFEEVMKDLNVTRAARLQLQKTQYLQLGQNRGQYYGGSLPNV | (SEQ ID NO. 30) |
| NQIGNGNIDLPFQVSNSVLDTSRTTRHHGLVERVYRDRNRISSPHRRPLSVDKHGRQRTNSDSALHQS | |
| AMNPKPHEVFAGGSQELQPKRLLLTVPGTEKSESNADKDSQEQSWDDKKSIFPSPDQELNPSVLPAAH | |
| NTGGSLPDLTNIQFPPPLSTPLDPEDTVTFPSLSSSNSTGSLTTNLTHLGISVASHGNNGEKNIFFLK | |
| TCTSCEDVYDFYFVGIPTSSQTTMTATAQRRQPPVVPLTLTSDLTLQQSPQQLSPTLSSPINITQSMK | |
| LSASSLQQYRNQTGSPATQSPTSPVSNQGFSPGSSPQPQHIPVVGSIFGDSFYDQQLALRQTNALSHQ | |
| VCEDGRRLEITHVRLSRLHAELCFCFSQLEQFNMIENPISSTSLYNQCSTLNYTQAAMMGLTGSSLQD | |
| SQQLGYGNHGNIPNIILTISVTGESPPSLSKELTNSLAGVGDVSFDPDTQFPLDELKIDPLTLDGLHM | |
| LNDPDMVLADPATEDTFRMDRL | |

Fugu CREAP1 DNA_sequence:

| | |
|---|---|
| ATGGCGTCCTCTAACAATCCTCGCAAATTTAGCGAAAAAATCGCACTGCATAACCAGAAACAAGCAGA | (SEQ ID No. 31) |
| GGAGACTGCTGCGTTCGAAGAAGTGATGAAGGACCTGAACGTCACAAGGGCTGCCCGGGTAAGACAGC | |
| TGCAGTTACAGAAGACCCAGTATTTGCAACTAGGGCAGAATCGTGGACAGTACTATGGAGGCTCACTG | |
| CCCAATGTCAATCAGATTGGAAATGGCAACATTGACCTGCCTTTTCAGGTGAGCAGGACAAACTCAGA | |
| CTCAGCTTTACATCAGAGTGCCATGAATCCAAAGCCCCACGAAGTGTTTGCTGGGGGGTCGCAGGAGC | |
| TGCAGCCCAAACGACTGCTGCTAACAGTGCCTGGAACCGAAAAATCGGAATCAAACGCAGACAAAGAT | |
| TCGCAGGAGCAGTCGTGGGATGACAAAAAGAGTATTTTTCCATCACCAGACCAGGAGTTAAACCCCTC | |
| CGTGCTTCCAGCCGCGCACAACACCGGCGGTTCGCTCCCCGACCTGACCAACATCCAGTTCCCTCCTC | |
| CACTGTCCACCCCACTGGACCCCGAGGACACCGTCACCTTCCCCTCCCTCAGCTCCTCTAACAGCACA | |
| GGCAGTCTGACTACCAACCTCACCCACCTGGGCATCAGTGTGGCCAGCCATGGTAATAACGGAGAGAA | |
| AAATATATTTTTTTTAAAAACATGCACTTCATGCGAGGATGTTAAATAATATTACGACTTTTATTTTG | |
| TAGGGATTCCCACTTCCTCTCAAACCACCATGACAGCAACAGCACAGCGGCGGCAACCACCCGTGGTC | |
| CCCCTCACCCTCACCTCTGACCTGACTCTTCAACAGTCCCCCCAGCAGCTTTCACCCACCCTCTCCTC | |
| ACCCATTAACATCACACAGAGCATGAAGCTTAGTGCTAGCTAACATTCTTCCCTCCAACAGTACCGCA | |
| ATCAGACTGGCTCACCAGCCACTCAGTCTCCAACCTCCCCAGTCTCCAATCAAGGCTTCTCCCCCGGC | |
| AGCTCGCCTCAACCACAGCACATTCCTGTGGTGGGCAGTATATTTGGGGACTCCTTCTATGATCAGCA | |
| GTTGGCTCTGAGGCAGACCAATGCCCTTTCTCATCAGGTGTGTGAGGACGGCCGCAGGTTAGAAATAA | |
| CACACGTACGTCTCTCACGACTTCACGCCGAGCTTTGTTTTTGTTTTTCTCAGCTGGAGCAGTTCAAT | |
| ATGATAGAGAACCCCATCAGCTCCACCAGCCTGTACAATCAGTGCTCCACCCTTAATTACACACAGGC | |
| AGCCATGATGGGCCTCACCGGGAGCAGCCTGCAGGACTCGCAGCAGCTCGGCTACGGCAATCACGGCA | |
| ACATCCCCAACATCATACTGACAATTTCAGTCACAGGGGAGTCTCCGCCGAGCCTCTCCAAAGAGCTG | |
| ACCAACTCATTGGCCGGCGTCGGCGACGTCAGCTTTGATCCAGACACGCAGTTTCCTCTGGACGAGCT | |
| GAAGATCGACCCGCTGACCTTGGACGGCCTGCACATGCTCAACGACCCAGACATGGTGCTGGCAGACC | |
| CCGCCACAGAGGACACGTTCAGGATGGACAGGCTGTAA | |

### Example 13

### Comparison of the Human CREAP Coding Regions with other CREAP Proteins from other Species

All three CREAP sequences were compared first by a global alignment of their coding regions as shown in FIG. 1. Each protein is of similar size, with CREAP2 being somewhat larger (693 amino acids compared to 650 and 619 amino acids in CREAP1 and 3, respectively). The proteins can be divided into roughly 3 domains based on conservation. The first is a conserved amino terminal third with a high degree of identity through amino acid 267 of CREAP1 (i.e., amino acids 1-267). This region is roughly 33% identical between all three CREAPs. The second domain is a central region spanning through amino acids 289-538 of CREAP1 that is highly enriched in runs of proline, glycine and serine residues. This corresponds to amino acids 289-529, 376-606, 235-533 of CREAP1 CREAP2, and CREAP3 respectively. This region has little amino acid identity but is similar in amino acid composition. Finally, the carboxy terminal third of the protein (roughly corresponding to the last 78 amino acids of CREAP1 (amino acids 575-650 of CREAP1) are again highly conserved with 38% amino acid identities in all three proteins.

Interestingly, the most conserved part of the protein is the amino terminus. A region of 80% identity over 24 amino acids exists in all three proteins. This region is also conserved in Drosophila and is essential for CREAP function and likely represents a key region regulating CREAP function. The conservation of the amino terminal end of CREAPs suggests that this region is critical to its function. This idea is supported by data which shows that deletion of the amino-terminal 250 amino acids destroys CREAP1 activity (see Table 1 above).

To further identify if the most amino terminal residues were critical, a deletion of the most N-terminal 59 amino acids in CREAP1 was produced. CREAP1 cDNA was excised from the original pCMV-SPORT6 plasmid with Scal/Xhol restriction enzymes (Roche Applied Science, Indianapolis, Ind., USA). The Scal digested CREAP1 cDNA fragment of 2382 nt which deleted 177 nucleotides of the CREAP1 ORF was gel purified and subcloned in frame into EcoRV/SalI digested pFLAG-CMV6B vector (BD Biosciences). Correct clones were isolated and sequence verffied according to conventional methods. This protein (delta59) was tested in promoter-reporter assay. Consistent with its conservation, deletion of these residues resulted in an 80-90% loss of CREAP activity (data not shown).

The similarity of human CREAP1 and homologs from other species are shown in FIG. 1. Overall, the 3 domains described for human CREAPs are also contained in the other CREAP sequences. The amino terminal end is highly conserved. Notably, the conserved amino acids at the very amino-terminal of the human CREAPs is also highly conserved in these proteins.

The human CREAP1 cDNA identified in this study encodes a predicted 650 amino acid protein. The cDNA is partially overlapping with a number of cDNAs annotated as KIAA0616 but differs in the predicted c-terminal end of the encoded protein. We were able to identify N-terminal coil-coil domain (amino acids 8-54), serine/glutamine-rich domain (amino acids 289-559) and strong negatively charged C-terminal domain (amino acids 602-643) (data not shown). Along with human CREAP2 and CREAP3, genes encoding proteins highly similar to CREAP1 were found in the mouse and Fugu genomes as shown. Overall the human and mouse CREAP1 genes are 90% identical. The predicted Fugu protein is 566 amino acids long and is 66% identical to human CREAP1.

We also have identified a CREAP1 like gene predicted in the Drosophila genome. While the mammalian and fish CREAP1 genes are only about 20% identical with the Drosophila sequence, the Drosophila sequence shares a similar organization to the other CREAP1 proteins. Each protein contains highly conserved amino and carboxyl terminal regions and a central domain rich in proline, glutamine and serine residues. We have termed the Drosophila predicted gene dCREAP. The first 22 of 28 amino acids of dCREAP are identical with human CREAP1. The amino-terminus has an absolutely conserved consensus PKA or PKC consensus phosphorylation site (RKFS) similar to the phosphorylation site in CREB proteins. Phosphorylation of this serine in CREB (serine 133 in CREB1) is required for induction of CREB dependent gene expression by cAMP.

The first 32 amino acids of dCREAP are 69% and 84% identical and similar, respectively to human CREAP1 again supporting the idea that the amino terminal end of CREAPs are critical to their function. The central domain of dCREAP is again a lower complexity region with little homology. Although the predicted dCREAP coding region does have some glycine and proline rich regions, it is unique in being highly rich in glutamine residues. Again, similar to the human CREAPs, the very carboxy terminus of the protein is highly conserved with human CREAP1 (30% over the last 30 amino acids).

The relatedness of the CREAP genes are shown in Table 4. Overall, the human CREAP genes are more related to each other than to dCREAP. CREAP2 is slightly more similar to CREAP1 and CREAP3 than are CREAP1 and CREAP3 to each other, but all are between 34-39% identical. All human CREAPs were found to be about 20% identical to the predicted dCREAP gene, although the similarity as shown above is largely due to the highly conserved amino and carboxy ends of the proteins. It should be noted that all three CREAP genes are highly conserved in the mouse and human genomes (data not shown). This suggests that the individual isoforms have unique and critical functions. The evolutionary conservation of CREAP supports the notion that CREAP is a critical regulator of CRE activity.

TABLE 4
Amino acid similarity of CREAP genes from various species.

| | HCREAP1 | HCREAP2 | HCREAP3 | MCREAP1 | FCREAP1 | dCREAP |
|---|---|---|---|---|---|---|
| HCREAP1 | | 32 | 32 | 89 | 63 | 19 |
| HCREAP2 | | | 33 | 34 | 31 | 18 |
| HCREAP3 | | | | 30 | 60 | 15 |
| MCREAP1 | | | | | 60 | 21 |
| FCREAP1 | | | | | | 20 |
| dCREAP | | | | | | |

Numbers shown represent the percentage identical amino acids throughout the entire protein coding region and were calculated as described above. The percent identity is based on an automated alignment using ClustalW V1.74

### Example 14

### Activity of CREAP2 and CREAP3

The homology between the human CREAP genes suggests that they are functionally related. To investigate this, the ability of CREAP2 and CREAP3 to activate gene expression driven by the IL-8 promoter and a CRE-dependent promoter was tested in co-transfection assays as disclosed above. Briefly, the levels of expression of a luciferase reporter driven by either the IL-8 promoter or a minimal promoter linked to 4 copies of CRE were determined after cotransfection with either an empty pCMV-SPORT6 expression vector or with the same vector carrying a cDNA for CREAP1, CREAP2 or CREAP3. Results indicate that cotransfection of CREAP1 with either an IL-8 promoter-dependent or CRE-dependent driven firefly luciferase gene resulted in a dramatic increase in luciferase activity (see Table 5). Transfection of either CREAP2 or CREAP3 also produced similar activation of both reporters. Other experiments showed that this activity is dependent upon the integrity of the CRE or the CRE-like site present in the IL-8 promoter (data not shown). Interestingly, CREAP3 has consistently shown a 2-4 fold higher level of induction of gene expression compared to CREAP1 and CREAP2. Thus, CREAP2 and CREAP3 are potent activators of CRE driven gene expression and the CREAP family represents a family of both conserved sequence and activity. In addition, all three CREAP family members have shown the ability to activate CREB-GAL4 fusion protein when overexpressed in a HLR-CREB cell line (Stratagene) carrying a genome-integrated UAS-Luc reporter supporting the evidence that CREAP proteins might induce gene expression through the interaction with CREB protein bound to the promoter (data not shown).

TABLE 5
Induction of a CRE driven promoter or the Interleukin-8 promoter by the
CREAP gene family.

| | CRE | IL-8 |
|---|---|---|
| Control | 1 | 1 |
| CREAP1 | 28.6 | 175.8571 |
| CREAP2 | 38.6 | 126.8571 |
| CREAP3 | 71.4 | 574.5714 |

Luciferase expression constructs driven by a minimal promoter linked to multiple copies of CRE or the Interleukin-8 promoter were cotransfected with an empty vector (control) or expression vectors encoding the three CREAP genes. The level of expression of luciferase is indicated relative to that obtained with cotransfection of the empty vector.

### Example 15

### CREAP1 Proteins are Transcription Activators

Several observations suggest that CREAP1 is a transcription co-activator. First, while we have been unable to identify any DNA binding activity in CREAP1, each CREAP protein contains a predicted N-terminal coil-coil domain (hCREAP1 residues 8-54), a serine/glutamine-rich domain (hCREAP1 residues 289-559) and a negatively charged carboxyl-terminus.

To determine if CREAP proteins can act as a transcription activators, various regions of all 3 CREAP homologs (amino acids 300-650 of CREAP1, amino acids 296-694 of CREAP2 and amino acids 335-635 of CREAP3) were expressed as fusion proteins with the DNA binding domain of GAL4 and tested for the ability to activate expression of a reporter gene linked to GAL4 protein binding sequences (UAS-Luc (pFR-Luc reporter). Briefly, the indicated regions of CREAP1, CREAP2 and CREAP3 were amplified by PCR and subcloned in frame into pCMV-BD vector (Stratagene) encoding GAL4 DNA binding domain. Selected plasmids and empty vector (pCMV-SPORT6) were transfected into HEK 293 cells at 75 ng/well using Fugene6 transfection reagent (Roche) as described above. pFR-Luc reporter (Stratagene) encoding firefly luciferase gene driven by minimal promoter linked to 5 concatamerized GAL4 binding sites (UAS) was co-transfected at 100 ng/well. As a positive control, the reporter was also co-transfected with a plasmid encoding GAL4-CREB fusion protein (Stratagene) alone or in the presence of pFC-PKA an expression construct encoding catalytic subunit of protein kinase A (Stratagene) to activate the CREB kinase inducible activation domain. Fold induction was compared to the reporter's activity measured in the cells transfected with pCMV-BD an expression vector carrying GAL4 DNA binding domain only. While the activity of the reporter was not significantly affected by the three full length CREAP proteins, the fusions containing the carboxy-terminal half of CREAP 1-3 potently induced expression of the UAS-Luc See Table 6.

TABLE 6
Demonstration that CREAP1 proteins are trancription activators.

| | Fold | |
|---|---|---|
| | Induction | STDEV |
| Vector | 1.00 | 0.21 |
| CREAP1 | 2.467531 | 1.478808 |
| CREAP2 | 2.47 | 0.41 |
| CREAP3 | 1.58 | 0.74 |
| GALA-CREAP1.1 | 2692.60 | 556.19 |
| GAL4-CREAP2.1 | 1373.88 | 222.52 |
| GAL4-CREAP3.1 | 1364.17 | 263.62 |
| GAL4-CREB | 7.66 | 0.34 |
| GAL4-CREB/PKA | 351.4352 | 11.52481 |

Expression constructs encoding full length CREAP1, CREAP2 and CREAP3 as well as a Gal4 DNA binding domain alone or fused with C-terminal portions of CREAP1, CREAP2 and CREAP3 were tested for the ability to induce expression of a luciferase gene controled by a minimal promoter linked to GAL4 DNA binding sites (pFRLuciferase). The data shown are normalized to the value seen with pCMV-BD vector co-transfected with pFR-Luc reporter.

To determine if CREAP proteins can directly activate CREB1 protein, the expression constructs of CREAP1, CREAP2 and CREAP3 were transfected individually or with GAL4-CREB plasmid (Stratagene) into HLR cell line (Stratagene) carrying genomic DNA integrated copies of pFR-Luc reporter. Briefly, HLR cells were maintained per manufacturer's instructions. Selected plasmids and either empty vector (pCMV-BD) or GAL4-CREB plasmid were transfected at 75 ng/well using Fugene6 transfection reagent (Roche) as described above. As a positive control pFC-PKA an expression construct encoding catalytic subunit of protein kinase A (Stratagene) was also co-trasfected with GAL4-CREB. Fold of activation was compared to the reporter's activity measured in the cells transfected with empty vector. While the activity of the reporter was not significantly affected by the three full length CREAP proteins, the activity of GA4L-CREB fusion protein was upregulated when co-transfected with the three full length CREAPs suggesting that CREB and CREAP proteins interact to form active transcriptional complex. See Tables 7 below.

TABLE 7
CREAP1 acts by activating CREB.

| | Fold | |
|---|---|---|
| | Induction | STDEV |
| pCMV-SPORT6 | 1 | 1.73205081 |
| GAL4-CREB | 75.54687245 | 4.42421391 |
| GAL4-CREB/PKA | 676.3531756 | 6.86497848 |
| CREAPI | 6.122284386 | 3.1169132 |
| GAL4-CREB/CREAP1 | 233.1430292 | 33.1345737 |
| CREAP2 | 2.435298629 | 2.05959793 |
| GAL4-CREB/CREAP2 | 177.5539854 | 23.0678772 |
| CREAP3 | 2.457796272 | 2.42452624 |
| GAL4-CREBICREAP3 | 447.635808 | 36.439389 |

The ability of full length CREAP1, CREAP2 and CREAP3 to induce the activity of GAL4-CREB fusion protein (Stratagene) was tested. The data presented are normalized to the value seen with pCMV-BD vector. All CREAPs and PKA significantly induced GAL4-CREB mediated activation. Note the fold induction obtained with positive control is lower when compared to the data from Table 6 when all the plasmids including the reporter were transiently transfected.

To determine if CREAP1 can interact directly with CREB, K1 and K5 variants of CREAP1 (see Table 1) were transfected into HEK293 cell grown in 100 mm dishes (Falcon) using Fugene6 reagent (Roche Applied Science) according to the protocol provided by the manufacturer. 40 hours after transfection, cells were scraped from the plates in PBS and lysed in 800 μl of Low Stringency buffer containing: 10 mM HEPES pH 7.6, 250 mM NaCl, 5 mM EDTA, 1 mM DTT, 0.1% NP-40 and freshly dissolved protease inhibitors. Immuno-precipitation was carried out using M2-agarose beads (Sigma). Precipitated proteins were separated on 4-20% of SDS-PAGE (Invitrogen) and transferred to nitrocellulose membrane (Invitrogen). Western blot were performed using antibody against CREB (Cell Signaling Technology). As a negative control expression construct encoding FLAG-tagged human histone deacetylase 1 (HDAC1) was used. We found that the N-terminal 170 amino acids fragment of CREAP1, containing the highly conserved coil-coil domain, was associated with endogenous CREB1 in vivo. Data shown in Table 1 demonstrate this region is absolutely essential for CREAP-mediated activation of CREs.

The CREAP family may represent an evolutionary conserved branch of CREB coactivators in addition to the recently identified LIM-only protein family (Fimia, G. et al. 2000, Mol Cell Biol 20, 8613-8622). Interestingly, while LIM-only protein associates with CREM a known CRE repressor and provides an activation function which is independent of phosphorylation and CBP, our data suggests that CREAP might interact with CREB1 bound to canonical CRE site and CREB2 bound to CRE-like element not recognized by CREB1 and thus activate expression of different pools of gene targets. Moreover, CREAP1 appears to allow synergy between proteins apparently bound to CREs and AP-1 binding sites. Elucidation of CREAP1 action should shed light on the mechanisms governing the tissue selective responses to activators of CREB.

The experiments described here raise the obvious question of the importance of the CRE-like site in regulating IL-8 expression during disease. While no CRE or CRE-like site was previously demonstrated to reside in the IL-8 promoter, β₂-adrenergic agonists (β₂-AR), which act to increase intracellular cAMP levels, induce IL-8 secretion in airway smooth muscle cells (Kavelaars A. et al. J. Neuroimmunol. 1997 August; 77(2):211-6). This is particularly important as the use of β₂-AR agonists as bronchodilators can exacerbate asthma and should be used in conjunction with anti-inflammatory steroids (Cockcroft, D. et al., 1993; Lancet 342:833-837; Knox, A. J 2002; Curr. Pharm Des. 1863-1869; Vathenen et al., 1988 Lancet 1:554-558)}. The data presented suggests that this effect may be directly due to activation of IL-8 transcription through the CRE-like site and perhaps CREAP1.

1. A method to prevent, treat or ameliorate pathological conditions related to abnormal activation of CRE-dependent gene expression or abnormal activation of chemokines comprising administering to a subject in need thereof an effective amount of a CREAP modulator.

2. The method of claim 1 wherein said pathological condition is a neurodegenerative disease.

3. The method of claim 1 wherein said pathological condition is an autoimmune disease.

4. The method of claim 1 wherein said pathological condition is an inflammatory disease.

5. The method of claim 1 wherein said pathological condition is selected from the group consisting of Alzheimer's Disease, Parkinson's disease, Huntington disease, osteoarthritis, psoriasis, asthma, CORD, rheumatoid arthritis, cancer, diabetes, hypertension and chronic pain.

6. The method of claim 1 wherein said CREAP modulator inhibits the activity of any one or more CREAP proteins selected from the group consisting of CREAP1, CREAP2 or CREAP3.

7. The method of claim 6 wherein said CREAP modulator comprises one or more antibodies to a CREAP protein, or fragments thereof, wherein said antibodies or fragments thereof can inhibit the activity of said CREAP protein.

8. The method of claim 6 wherein said modulator comprises one or more peptide mimetics to a CREAP protein wherein said peptide mimic can inhibit the activity of said CREAP protein.

9. The method of claim 1 wherein said CREAP modulator inhibits the expression of any one or more CREAP proteins selected from the group consisting of CREAP1, CREAP2 or CREAP3.

10. The method of claim 9 wherein said CREAP modulator comprises any one or more substances selected from the group consisting of antisense oligonucleotides, triple helix DNA, ribozymes, RNA aptamers and double or single stranded RNA wherein said substances are designed to inhibit the expression of a CREAP protein.

11. A method to prevent, treat or ameliorate pathological conditions related to abnormal activation of CRE-dependent gene expression or abnormal activation of chemokines comprising administering to a subject in need thereof a pharmaceutical composition comprising an effective amount of a CREAP modulator.

12. The method of claim 11 wherein said pathological condition is a neurodegenerative disease.

13. The method of claim 11 wherein said pathological condition is an autoimmune disease.

14. The method of claim 11 wherein said pathological condition is an inflammatory disease.

15. The method of claim 11 wherein said pathological condition is selected from the group consisting of Alzheimer's Disease, Parkinson's disease, Huntington disease, osteoarthritis, psoriasis, asthma, COPD, rheumatoid arthritis, cancer, diabetes, hypertension and chronic pain.

16. The method of claim 11 wherein said CREAP modulator inhibits the activity of any one or more CREAP proteins selected from the group consisting of CREAP1, CREAP2 or CREAP3.

17. The method of claim 11 wherein said CREAP modulator comprises one or more antibodies to a CREAP protein, or fragments thereof, wherein said antibodies or fragments thereof can inhibit the activity of said CREAP protein.

18. The method of claim 11 wherein said CREAP modulator comprises one or more peptide mimetics to a CREAP protein wherein said peptide mimic can inhibit the activity of said CREAP protein.

19. The method of claim 11 wherein said CREAP modulator inhibits the expression of any one or more CREAP proteins selected from the group consisting of CREAP1, CREAP2 or CREAP3.

20. The method of claim 19 wherein said CREAP modulator comprises any one or more substances selected from the group consisting of antisense oligonucleotides, triple helix DNA, ribozymes, RNA aptamers and double or single stranded RNA wherein said substances are designed to inhibit the expression of a CREAP protein.

21. A method to identify modulators useful to prevent, treat or ameliorate pathological conditions related to abnormal activation of CRE-dependent gene expression or abnormal chemokine activation comprising assaying for the ability of a candidate modulator to inhibit the activity of a CREAP protein.

22. The method of claim 21 wherein said CREAP protein is selected from the group consisting of CREAP1, CREAP2 or CREAP3.

23. The method of claim 21 wherein said method further comprises assaying for the ability of an identified CREAP inhibitory modulator to reverse the pathological effects observed in in vitro, ex vivo or in vivo models of said pathological conditions and/or in clinical studies with subjects with said pathological conditions.

24. The method of claim 21 wherein said pathological condition is a neurodegenerative disease.

25. The method of claim 21 wherein said pathological condition is an autoimmune disease.

26. The method of claim 21 wherein said pathological condition is an inflammatory disease.

27. The method of claim 21 wherein said pathological condition is selected from the group consisting of Alzheimer's Disease, Parkinson's disease, Huntington disease, osteoarthritis, psoriasis, asthma, COPD, rheumatoid arthritis, cancer, diabetes, hypertension and chronic pain.

28. A method to identify modulators useful to prevent, treat or ameliorate pathological conditions related to abnormal activation of CRE-dependent gene expression or abnormal chemokine activation comprising assaying for the ability of a candidate modulator to inhibit the expression of a CREAP protein.

29. The method of claim 28 wherein said CREAP protein is selected from the group consisting of CREAP1, CREAP2 or CREAP3.

30. The method of claim 28 wherein said method further comprises assaying for the ability of an identified CREAP inhibitory modulator to reverse the pathological effects observed in in vitro, ex vivo or in vivo models of said pathological conditions and/or in clinical studies with subjects with said pathological conditions.

31. The method of claim 28 wherein said pathological condition is a neurodegenerative disease.

32. The method of claim 28 wherein said pathological condition is an autoimmune disease.

33. The method of claim 28 wherein said pathological condition is an inflammatory disease.

34. The method of claim 28 wherein said pathological condition is selected from the group consisting of Alzheimer's Disease, Parkinson's Disease, Huntington Disease, osteoarthritis, psoriasis, asthma, CORD, rheumatoid arthritis, cancer, diabetes, hypertension and chronic pain.

35. A pharmaceutical composition comprising one or more CREAP modulators in an amount effective to prevent, treat or ameliorate a pathological condition related to abnormal activation of CRE-dependent gene expression or abnormal chemokine activation in a subject in need thereof.

36. The pharmaceutical composition according to claim 35 wherein said pathological condition is a neurodegenerative disease.

37. The pharmaceutical composition according to claim 35 wherein said pathological condition is an autoimmune disease.

38. The pharmaceutical composition according to claim 35 wherein said pathological condition is an inflammatory disease.

39. The pharmaceutical composition according to claim 35 wherein said pathological condition is selected from the group consisting of Alzheimer's Disease, Parkinson's disease, Huntington disease, osteoarthritis, psoriasis, asthma, COPD, rheumatoid arthritis, cancer, diabetes, hypertension and chronic pain.

40. The pharmaceutical composition according to claim 35 wherein said CREAP modulator inhibits the activity of any one or more CREAP proteins selected from the group consisting of CREAP1, CREAP2 or CREAP3.

41. The pharmaceutical composition of claim 40 wherein said CREAP modulator comprises one or more antibodies to a CREAP protein, or fragments thereof, wherein said antibodies or fragments thereof can inhibit the activity of said CREAP protein.

42. The pharmaceutical composition of claim 40 wherein said CREAP modulator comprises one or more peptide mimetics to a CREAP protein wherein said peptide mimic can inhibit the activity of said CREAP protein.

43. The pharmaceutical composition according to claim 35 wherein said CREAP modulator inhibits the expression of any one or more CREAP proteins selected from the group consisting of CREAP1, CREAP2 or CREAP3.

44. The pharmaceutical composition of claim 43 wherein said CREAP modulator comprises any one or more substances selected from the group consisting of antisense oligonucleotides, triple helix DNA, ribozymes, RNA aptamer and double or single stranded RNA wherein said substances are designed to inhibit CREAP gene expression.

45. A method to diagnose subjects suffering from pathological conditions related to abnormal activation of CRE-dependent gene expression or abnormal chemokine activation and who nary be suitable candidates for treatment with CREAP modulators comprising assaying mRNA levels of a CREAP protein in a biological sample from said subject wherein a subject with increased mRNA levels compared to controls would be a suitable candidate for CREAP modulator treatment.

46. The method of claim 45 wherein said CREAP protein is selected from the group consisting of CREAP1, CREAP2 or CREAP3.

47. A method to diagnose subjects suffering from pathological conditions related to abnormal activation of CRE-dependent gene expression or abnormal activation of chemokines who may be suitable candidates for treatment with CREAP modulators comprising detecting levels of CREAP protein in a biological sample from said subject wherein subjects with increased levels compared to controls would be suitable candidates for CREAP modulator treatment.

48. The method of claim 47 wherein said CREAP protein is selected from the group consisting of CREAP1, CREAP2 or CREAP3.

49. A method to prevent, treat or ameliorate pathological conditions related to abnormal activation of CRE-dependent gene expression or abnormal activation of chemokines comprising:
(a) assaying for CREAP mRNA and/or protein levels in a subject; and,
(b) administering to a subject with increased levels of CREAP mRNA and/or protein levels compared to controls a CREAP modulator in an amount sufficient to prevent, treat or ameliorate said pathological conditions.

50. The method of claim 49 wherein said pathological condition is a neurodegenerative disease.

51. The method of claim 49 wherein said pathological condition is an autoimmune disease.

52. The method of claim 49 wherein said pathological condition is an inflammatory disease.

53. The method of claim 49 wherein said pathological condition is selected from the group consisting of Alzheimer's Disease, Parkinson's disease, Huntington disease, osteoarthritis, psoriasis, asthma, COPD, rheumatoid arthritis, cancer, diabetes, hypertension and chronic pain.

54. A diagnostic kit for detecting mRNA levels and/or protein levels of a CREAP protein in a biological sample, said kit comprising:
(a) a polynucleotide of CREAP or a fragment thereof;
(b) a nucleotide sequence complementary to that of (a);
(c) a CREAP polypeptide, or a fragment thereof;
(d) an antibody to a CREAP polypeptide; or
(e) a peptide mimic to a CREAP protein
wherein components (a), (b), (c), (d) or (e) may comprise a substantial component.

55. The method of claim 54 wherein said CREAP protein is selected from the group consisting of CREAP1, CREAP2 or CREAP3.

56. An isolated polypeptide comprising a CREAP amino acid sequence selected from the group consisting of SEQ ID NOs:2,16 and 25.

57. An isolated nucleic acid sequence comprising a nucleic acid sequence that encodes a polypeptide of claim 56.

58. An isolated polypeptide consisting of a CREAP amino acid sequence selected from the group consisting of SEQ ID NOs: 2,16 and 25.

59. An isolated nucleic acid sequence comprising a nucleic acid sequence that encodes a polypeptide of claim 58.

60. An isolated CREAP polypeptide encoded by a CREAP gene of an organism.

61. An isolated DNA comprising a nucleic acid sequence that encodes the CREAP polypeptide of claim 60.

62. A vector molecule comprising a fragment of the isolated nucleic acid according to claim 57.

63. The vector molecule according to claim 62 comprising any one or more transcriptional control sequence.

64. A host cell comprising the vector molecule according to claim 63.

65. An antibody or a fragment thereof which specifically binds to a polypeptide that comprises the amino acid sequence set forth in claim 56 or to a fragment of said polypeptide.

66. An antibody fragment according to claim 65 which is an Fab or F(ab′)₂ fragment.

67. An antibody according to claim 65 which is a polyclonal antibody.

68. An antibody according to claim 65 which is a monoclonal antibody.

69. A method for producing a polypeptide as defined in claim 56, comprising culturing a host cell having incorporated therein an expression vector comprising an exogenously-derived polynucleotide encoding a polypeptide comprising an amino acid sequence selected from the group consisting of SEQ ID NOs:2,16 and 25 under conditions sufficient for expression of the polypeptide in the host cell, thereby causing the production of the expressed polypeptide.

70. The method according to claim 69, said method further comprises recovering the polypeptide produced by said cell.

71. The method according to claim 69 wherein said exogenously-derived polynucleotide comprises the nucleotide sequence selected from the group consisting of SEQ ID NOs: 1,15 and 24.

72. A method for producing a polypeptide as defined in claim 56, comprising culturing a host cell having incorporated therein an expression vector comprising an exogenously-derived polynucleotide encoding a polypeptide consisting of an amino acid sequence selected from the group consisting of SEQ ID NOs:2, 16 and 25 under conditions sufficient for expression of the polypeptide in the host cell, thereby causing the production of the expressed polypeptide.

73. The method according to claim 72, said method further comprising recovering the polypeptide produced by said cell.

74. The method according to claim 72, wherein said exogenously-derived polynucleotide comprises the nucleotide sequence selected from the group consisting of SEQ ID NOs: 1,15 and 24.

75. A vector molecule comprising a nucleic acid sequence selected from the group consisting of nucleic acid sequences encoding human CREAP protein fragments of amino acid regions 1-267, 289-538, 356-580 and 575-650.
